(19) United States
(12) Patent Application Publication
(10) Pub. No.: US 2009/0099158 A1
(43) Pub. Date: Apr. 16, 2009

(54) TETRAHYDROISOQUINOLINE COMPOUNDS AS MODULATORS OF THE HISTAMINE H3 RECEPTOR

(76) Inventors: Cheryl A. Grice, Carlsbad, CA (US); Michael A. Letavic, San Diego, CA (US); Alejandro Santillan, JR., San Diego, CA (US); Kimberly L. Schwarz, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/039,162

(22) Filed: Feb. 28, 2008

Related U.S. Application Data
(60) Provisional application No. 60/892,324, filed on Mar. 1, 2007.

(51) Int. Cl. A61K 31/4725; C07D 401/02; A61K 31/551; A61K 31/496; A61P 25/00; C07D 471/04; A61K 31/4985; A61K 31/5377; C07D 413/02

(52) U.S. Cl. 514/218; 540/575; 514/253.05; 544/363; 544/349; 514/249; 514/235.2; 544/128; 546/146; 514/307

(57) ABSTRACT

Certain substituted tetrahydroisoquinoline compounds are histamine H₃ receptor modulators useful in the treatment of histamine H₃ receptor-mediated diseases.

### CROSS-REFERENCE TO RELATED APPLICATIONS

This application claims the benefit of U.S. Provisional Application 60/892,324, filed Mar. 1, 2007, which is hereby incorporated by reference in its entirety.

### FIELD OF THE INVENTION

The present invention relates to certain tetrahydroisoquinoline compounds, pharmaceutical compositions containing them, and methods of using them for the treatment of disease states, disorders, and conditions mediated by the histamine H₃ receptor.

### BACKGROUND OF THE INVENTION

The histamine H₃ receptor was first described as a presynaptic autoreceptor in the central nervous system (CNS) (Arrang, J.-M. et al. Nature 1983, 302, 832-837) controlling the synthesis and release of histamine. The histamine H₃ receptor is primarily expressed in the mammalian central nervous system (CNS), with some minimal expression in peripheral tissues such as vascular smooth muscle.

Thus, several indications for histamine H₃ antagonists and inverse agonists have been proposed based on animal pharmacology and other experiments with known histamine H₃ antagonists (e.g. thioperamide). (See: Krause et al. and Phillips et al. in “The Histamine H₃ Receptor-A Target for New Drugs”, Leurs, R. and Timmerman, H., (Eds.), Elsevier, 1998, pp. 175-196 and 197-222; Morisset, S. et al. Nature 2000, 408, 860-864.) These include conditions such as cognitive disorders, sleep disorders, psychiatric disorders, and other disorders.

For example, histamine H₃ antagonists have been shown to have pharmacological activity relevant to several key symptoms of depression, including sleep disorders (e.g. sleep disturbances, fatigue, and lethargy) and cognitive difficulties (e.g. memory and concentration impairment), as described above. For reviews, see: Celanire, S. Drug Discovery Today 2005, 10(23/24), 1613-1627; Hancock, A. A. Biochem. Pharmacol. 2006, 71, 1103-1113; Bonaventure, P. et al. Biochem. Pharm. 2007, 73, 1084-1096; and Letavic, M. A. et al. Prog. Med. Chem. 1996, 44, 181-206. There remains a need for potent histamine H₃ receptor modulators with desirable pharmaceutical properties.

Tetrahydroisoquinoline hydroxamic acids have been described in Intl. Pat. Appl. Publ. WO 2005/108367. Tetrahydroisoquinoline benzoic acid derivatives are described as PPAR receptor antagonists in Intl. Pat. Appl. Publ. WO 01/12187. Tetrahydroisoquinoline bis amides are described in Intl. Pat. Appl. Publ. WO 96/29309. Tetrahydroisoquinolines as modulators of the histamine H₃ receptor and serotonin transporter have been described in Intl. Pat. Appl. Publ. WO 2006/066197 (equivalent of US Pat. Appl. Publ. US 2006/0194837) and WO 2006/138604 (equivalent of US Pat. Appl. Publ. US 2006/0293316), and naphthyridines as modulators of the histamine H₃ receptor and serotonin transporter have been described in Intl. Pat. Appl. Publ. WO 2006/138714 (equivalent of US Pat. Appl. Publ. US 2006/0287292). Tetrahydroisoquinolines have been described as histamine H₃ receptor antagonists in Intl. Patl Appl. Publ. WO 02/076925 and Intl. Pat. Appl. Publ. WO 2004/026837, and by Jesudason, C. D. et al. (Bioorg. Med. Chem. Lett. 2006, 16(13), 3415-3418).

### SUMMARY OF THE INVENTION

Certain tetrahydroisoquinoline derivatives have now been found to have histamine H₃ receptor modulating activity. Thus, the invention is directed to the general and preferred embodiments defined, respectively, by the independent and dependent claims appended hereto, which are incorporated by reference herein.

In one general aspect the invention relates to a compound of the following Formula (I):

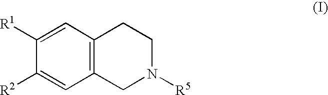

wherein
one of R¹ and R² is -L-N(R³)R⁴ and the other is —H;
   where L is C(O) or CH₂; and
   —N(R³)R⁴ is one of the following moieties:

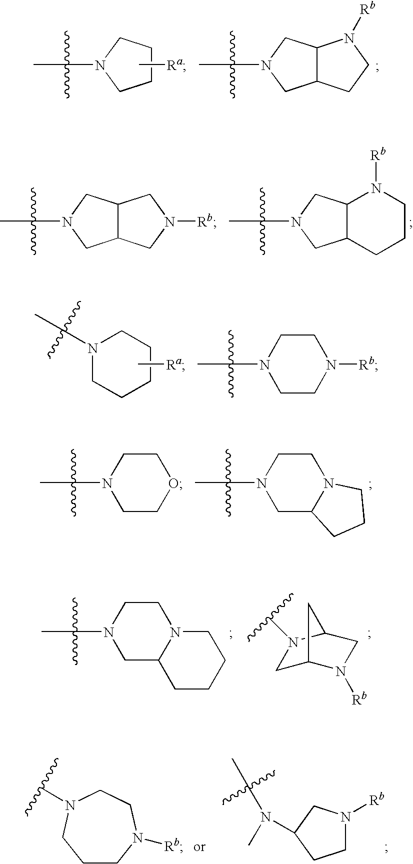

where R^{a} is —H, —C₁₋₄alkyl, —C₁₋₄alkyl-OH, —OH, —NR^{c}R^{d}, or —CH₂NR^{c}R^{d};
   R^{c} and R^{d} are each independently H or —C₁₋₄alkyl, or R^{c} and R^{d} taken together with the nitrogen to which they are attached form pyrrolidinyl, piperidinyl, or morpholinyl; and
R^{b} is —C₁₋₄alkyl or —C₃₋₇cycloalkyl;
R⁵ is —H, C₁₋₄alkyl, C₃₋₇cycloalkyl, —CH₂-phenyl, —CH₂-(monocyclic heteroaryl), —C(O)—C₁₋₄alkyl, —C(O)—C₃₋₇cycloalkyl, —C(O)-(monocyclic heterocycloalkyl), —C(O)-phenyl, —C(O)-(monocyclic heteroaryl), —C(O)CH₂—C₃₋₇cycloalkyl, —C(O)CH₂-phenyl, —C(O)CH₂-(monocyclic heteroaryl), —CO₂C₁₋₄alkyl, —SO₂C₁₋₄alkyl, or —SO₂-phenyl;
   where each cycloalkyl, phenyl, monocyclic heteroaryl, or monocyclic heterocycloalkyl group in R⁵ is unsubstituted or substituted with one or two substituents independently selected from the group consisting of —C₁₋₄alkyl, —CF₃, halo, —CN, —NO₂, —OH, —OC₁₋₄alkyl, —C₃₋₇cycloalkyl, and —NR^{x}R^{y};
   R^{x} and R^{y} are each independently H or —C₁₋₄alkyl;
      with the proviso that the compound of Formula (I) comprises at least one nitrogen atom which is not part of an amide, carbamoyl, cyano, nitro, or sulfonamide group;
      or a pharmaceutically acceptable salt, a pharmaceutically acceptable prodrug, or a pharmaceutically active metabolite thereof.

In a further general aspect, the invention relates to pharmaceutical compositions each comprising: (a) an effective amount of a compound of Formula (I), or a pharmaceutically acceptable salt, pharmaceutically acceptable prodrug, or pharmaceutically active metabolite thereof; and (b) a pharmaceutically acceptable excipient.

In another general aspect, the invention is directed to a method of treating a subject suffering from or diagnosed with a disease, disorder, or medical condition mediated by histamine H₃ receptor activity, comprising administering to the subject in need of such treatment an effective amount of a compound of Formula (I), or a pharmaceutically acceptable salt, pharmaceutically acceptable prodrug, or pharmaceutically active metabolite thereof.

In certain preferred embodiments of the inventive method, the disease, disorder, or medical condition is selected from: cognitive disorders, sleep disorders, psychiatric disorders, and other disorders.

Additional embodiments, features, and advantages of the invention will be apparent from the following detailed description and through practice of the invention.

### DETAILED DESCRIPTION

The invention may be more fully appreciated by reference to the following description, including the following glossary of terms and the concluding examples. For the sake of brevity, the disclosures of the publications, including patents, cited in this specification are herein incorporated by reference.

As used herein, the terms “including”, “containing” and “comprising” are used herein in their open, non-limiting sense.

The term “alkyl” refers to a straight- or branched-chain alkyl group having from 1 to 12 carbon atoms in the chain. Examples of alkyl groups include methyl (Me, which also may be structurally depicted by a bond “/”), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples.

The term “cycloalkyl” refers to a saturated or partially saturated, monocyclic, fused polycyclic, or spiro polycyclic carbocycle having from 3 to 12 ring atoms per carbocycle. Illustrative examples of cycloalkyl groups include the following entities, in the form of properly bonded moieties:

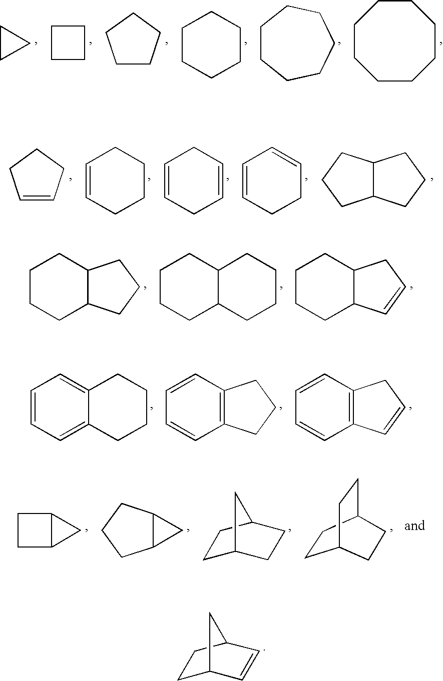

A “heterocycloalkyl” refers to a monocyclic ring structure that is saturated or partially saturated and has from 4 to 7 ring atoms per ring structure selected from carbon atoms and up to two heteroatoms selected from nitrogen, oxygen, and sulfur. The ring structure may optionally contain up to two oxo groups on sulfur ring members. Illustrative entities, in the form of properly bonded moieties, include:

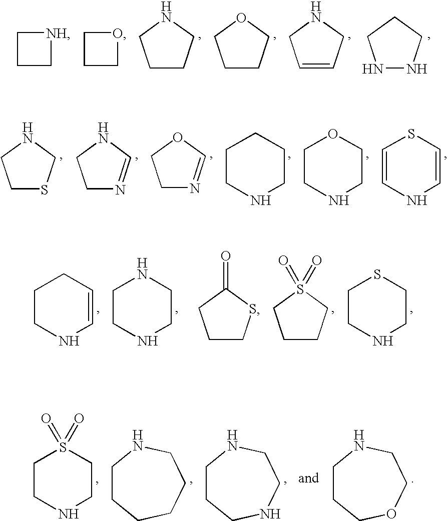

The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. Illustrative examples of heteroaryl groups include the following entities, in the form of properly bonded moieties:

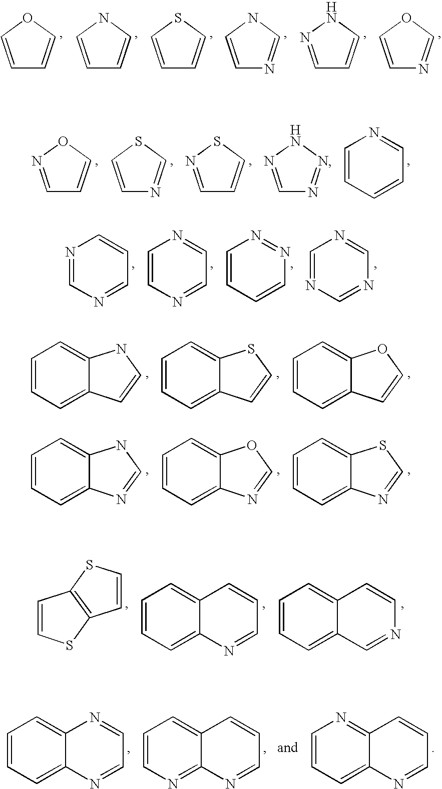

Those skilled in the art will recognize that the species of cycloalkyl, heterocycloalkyl, and heteroaryl groups listed or illustrated above are not exhaustive, and that additional species within the scope of these defined terms may also be selected.

The term “halogen” represents chlorine, fluorine, bromine or iodine. The term “halo” represents chloro, fluoro, bromo or iodo.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In cases where a specified moiety or group is not expressly noted as being optionally substituted or substituted with any specified substituent, it is understood that such a moiety or group is intended to be unsubstituted.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to embrace hydrates, solvates, and polymorphs of such compounds, and mixtures thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³²P, ³³P, ³⁵S, ¹⁸F, ³⁶Cl, and ¹²⁵I, respectively. Such isotopically labeled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the moiety for the variable appearing elsewhere. In other words, where a variable appears more than once, the choice of the species from a specified list is independent of the choice of the species for the same variable elsewhere in the formula.

In preferred embodiments of Formula (I), R¹ is -L-N(R³)R⁴ and R² is —H.

In preferred embodiments, L is C(O).

In preferred embodiments, —N(R³)R⁴ is one of the following moieties:

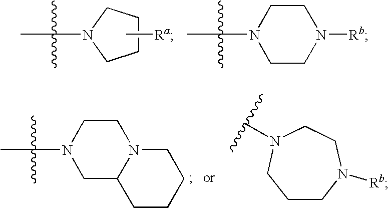

where R^{a} and R^{b} are as defined for Formula (I).

In preferred embodiments, R^{a} is —H, methyl, ethyl, isopropyl, tert-butyl, 1-hydroxy-1-methyl-ethyl, —OH, dimethylamino, piperidin-1-yl, morpholin-1-yl, or 2-pyrrolidin-1-ylmethyl.

In preferred embodiments, R^{b} is methyl, ethyl, isopropyl, tert-butyl, cyclopropyl, cyclobutyl, cyclopentyl, or cyclohexyl.

In preferred embodiments, —N(R³)R⁴ is 4-isopropyl-[1,4]diazepan-1-yl, piperidin-1-yl, morpholin-1-yl, 4-cyclopentyl-piperazin-1-yl, 4-cyclohexyl-piperazin-1-yl, octahydro-pyrido[1,2-a]pyrazin-2-yl, 4-cyclobutyl-piperazin-1-yl, 4-isopropyl-piperazin-1-yl, 4-cyclopropyl-piperazin-1-yl, 4-cyclobutyl-[1,4]diazepan-1-yl, 2-pyrrolidin-1-ylmethyl-pyrrolidin-1-yl, 4-(tetrahydro-furan-2-ylmethyl)-piperazin-1-yl, hexahydro-pyrrolo[1,2-a]pyrazin-2-yl, 4-dimethylamino-piperidin-1-yl, 3-dimethylamino-pyrrolidin-1-yl, [1,4′]bipiperidin-1′-yl, 4-morpholin-4-yl-piperidin-1-yl, N-methyl-N-(1-methyl-pyrrolidin-3-yl), 2-tert-butoxy-carbonyl-2,5-diaza-bicyclo[2.2.1]hept-5-yl, 1-tert-butoxy-carbonyl-hexahydro-pyrrolo[3,4-b]pyrrol-5-yl, 2-tert-butoxy-carbonyl-hexahydro-pyrrolo[3,4-c]pyrrol-5-yl, hexahydro-pyrrolo[3,4-c]pyrrol-2-yl, 2,5-diaza-bicyclo[2.2.1]hept-2-yl, hexahydro-pyrrolo[3,4-b]pyrrol-5-yl, 5-cyclobutyl-hexahydro-pyrrolo[3,4-c]pyrrol-2-yl, 5-cyclobutyl-2,5-diaza-bicyclo[2.2.1]hept-2-yl, 1-cyclobutyl-hexahydro-pyrrolo[3,4-b]pyrrol-5-yl, 4-tert-butyl-piperidin-1-yl, or 4-(1-hydroxy-1-methyl-ethyl)-piperidin-1-yl. In further preferred embodiments, —N(R³)R⁴ is 4-isopropyl-[1,4]diazepan-1-yl, octahydro-pyrido[1,2-a]pyrazin-2-yl, 4-cyclobutyl-piperazin-1-yl, 4-isopropyl-piperazin-1-yl, 4-cyclopropyl-piperazin-1-yl, 4-cyclobutyl-[1,4]diazepan-1-yl, or 2-pyrrolidin-1-ylmethyl-pyrrolidin-1-yl.

In preferred embodiments, R⁵ is —H, methyl, ethyl, propyl, or isopropyl. In further preferred embodiments, R⁵ is cyclopropyl, cyclobutyl, or cyclopentyl. In still further preferred embodiments, R⁵ is benzyl, thiophen-3-ylmethyl, or furan-3-ylmethyl. In still further preferred embodiments, R⁵ is acetyl, propionyl, butyryl, or 2,2-dimethylpropionyl. In still further preferred embodiments, R⁵ is cyclopropanecarbonyl, cyclobutanecarbonyl, cyclopentanecarbonyl, or cyclohexanecarbonyl. In still further preferred embodiments, R⁵ is tetrahydrofuran-2-carbonyl, tetrahydrofuran-3-carbonyl, or piperidine-4-carbonyl. In still further preferred embodiments, R⁵ is benzoyl, furan-3-carbonyl, or thiophen-3-carbonyl. In still further preferred embodiments, R⁵ is 2-cyclopentyl-acetyl, phenylacetyl, or 2-furan-2-yl-acetyl. In still further preferred embodiments, R⁵ is tert-butoxycarbonyl. In still further preferred embodiments, R⁵ is ethanesulfonyl, propane-1-sulfonyl, propane-2-sulfonyl, or benzenesulfonyl.

In certain preferred embodiments, the compound of Formula (I) is selected from the group consisting of:

| Ex. | Compound Name |
|---|---|
| 1 | 6-(4-Isopropyl-[1,4]diazepane-1-carbonyl)-3,4-dihydro-1H-isoquinoline-2- |
| | carboxylic acid tert-butyl ester; |
| 2 | 6-(4-Cyclopentyl-piperazine-1-carbonyl)-3,4-dihydro-1H-isoquinoline-2- |
| | carboxylic acid tert-butyl ester; |
| 3 | 6-(4-Cyclohexyl-piperazine-1-carbonyl)-3,4-dihydro-1H-isoquinoline-2- |
| | carboxylic acid tert-butyl ester; |
| 4 | 6-(Octahydro-pyrido[1,2-a]pyrazine-2-carbonyl)-3,4-dihydro-1H- |
| | isoquinoline-2-carboxylic acid tert-butyl ester; |
| 5 | (4-Isopropyl-[1,4]diazepan-1-yl)-(1,2,3,4-tetrahydro-isoquinolin-6-yl)-methanone; |
| 6 | Piperidin-1-yl-(1,2,3,4-tetrahydro-isoquinolin-6-yl)-methanone; |
| 7 | Morpholin-4-yl-(1,2,3,4-tetrahydro-isoquinolin-6-yl)-methanone; |
| 8 | (4-Cyclopentyl-piperazin-1-yl)-(1,2,3,4-tetrahydro-isoquinolin-6-yl)-methanone; |
| 9 | (4-Cyclohexyl-piperazin-1-yl)-(1,2,3,4-tetrahydro-isoquinolin-6-yl)-methanone; |
| 10 | (Octahydro-pyrido[1,2-a]pyrazin-2-yl)-(1,2,3,4-tetrahydro-isoquinolin-6- |
| | yl)-methanone; |
| 11 | (2-Benzyl-1,2,3,4-tetrahydro-isoquinolin-6-yl)-(4-isopropyl-[1,4]diazepan- |
| | 1-yl)-methanone; |
| 12 | (2-Benzyl-1,2,3,4-tetrahydro-isoquinolin-6-yl)-piperidin-1-yl-methanone; |
| 13 | (2-Benzyl-1,2,3,4-tetrahydro-isoquinolin-6-yl)-morpholin-4-yl-methanone; |
| 14 | (4-Cyclobutyl-piperazin-1-yl)-[2-(4-trifluoromethyl-benzyl)-1,2,3,4- |
| | tetrahydro-isoquinolin-6-yl]-methanone; |
| 15 | (4-Cyclobutyl-piperazin-1-yl)-(2-thiophen-3-ylmethyl-1,2,3,4-tetrahydro- |
| | isoquinolin-6-yl)-methanone; |
| 16 | (4-Cyclobutyl-piperazin-1-yl)-[2-(3,4-dichloro-benzyl)-1,2,3,4-tetrahydro- |
| | isoquinolin-6-yl]-methanone; |
| 17 | [2-(4-Chloro-benzyl)-1,2,3,4-tetrahydro-isoquinolin-6-yl]-(4-cyclobutyl- |
| | piperazin-1-yl)-methanone; |
| 18 | (2-Benzyl-1,2,3,4-tetrahydro-isoquinolin-6-yl)-(4-cyclobutyl-piperazin-1- |
| | yl)-methanone; |
| 19 | [2-(3,4-Dichloro-benzyl)-1,2,3,4-tetrahydro-isoquinolin-6-yl]-(4-isopropyl- |
| | piperazin-1-yl)-methanone; |
| 20 | (4-Isopropyl-piperazin-1-yl)-[2-(4-trifluoromethyl-benzyl)-1,2,3,4- |
| | tetrahydro-isoquinolin-6-yl]-methanone; |
| 21 | (2-Benzyl-1,2,3,4-tetrahydro-isoquinolin-6-yl)-(4-isopropyl-piperazin-1-yl)- |
| | methanone; |
| 22 | [2-(4-Chloro-benzyl)-1,2,3,4-tetrahydro-isoquinolin-6-yl]-(4-isopropyl- |
| | piperazin-1-yl)-methanone; |
| 23 | (4-Cyclopropyl-piperazin-1-yl)-[2-(4-trifluoromethyl-benzyl)-1,2,3,4- |
| | tetrahydro-isoquinolin-6-yl]-methanone; |
| 24 | [2-(4-Chloro-benzyl)-1,2,3,4-tetrahydro-isoquinolin-6-yl]-(4-cyclopropyl- |
| | piperazin-1-yl)-methanone; |
| 25 | 4-[6-(4-Cyclobutyl-piperazine-1-carbonyl)-3,4-dihydro-1H-isoquinolin-2- |
| | ylmethyl]-benzonitrile; |
| 26 | (2-Benzyl-1,2,3,4-tetrahydro-isoquinolin-7-yl)-(4-cyclobutyl-piperazin-1- |
| | yl)-methanone; |
| 27 | (2-Benzoyl-1,2,3,4-tetrahydro-isoquinolin-6-yl)-(4-cyclobutyl-piperazin-1- |
| | yl)-methanone; |
| 28 | (2-Benzoyl-1,2,3,4-tetrahydro-isoquinolin-6-yl)-(4-isopropyl-piperazin-1- |
| | yl)-methanone; |
| 29 | 1-[6-(4-Isopropyl-piperazine-1-carbonyl)-3,4-dihydro-1H-isoquinolin-2-yl]- |
| | ethanone; |
| 30 | 1-[6-(4-Cyclobutyl-piperazine-1-carbonyl)-3,4-dihydro-1H-isoquinolin-2- |
| | yl]-ethanone; |
| 31 | Cyclobutyl-[6-(4-cyclobutyl-piperazine-1-carbonyl)-3,4-dihydro-1H- |
| | isoquinolin-2-yl]-methanone; |
| 32 | [6-(4-Cyclobutyl-piperazine-1-carbonyl)-3,4-dihydro-1H-isoquinolin-2-yl]- |
| | cyclopentyl-methanone; |
| 33 | [6-(4-Cyclobutyl-piperazine-1-carbonyl)-3,4-dihydro-1H-isoquinolin-2-yl]- |
| | cyclohexyl-methanone; |
| 34 | [6-(4-Cyclobutyl-piperazine-1-carbonyl)-3,4-dihydro-1H-isoquinolin-2-yl]- |
| | cyclopropyl-methanone; |
| 35 | [6-(4-Cyclobutyl-[1,4]diazepane-1-carbonyl)-3,4-dihydro-1H-isoquinolin-2- |
| | yl]-phenyl-methanone; |
| 36 | [7-(4-Cyclobutyl-[1,4]diazepane-1-carbonyl)-3,4-dihydro-1H-isoquinolin-2- |
| | yl]-phenyl-methanone; |
| 37 | [7-(4-Cyclobutyl-[1,4]diazepane-1-carbonyl)-3,4-dihydro-1H-isoquinolin-2- |
| | yl]-cyclopentyl-methanone; |
| 38 | [7-(4-Cyclobutyl-[1,4]diazepane-1-carbonyl)-3,4-dihydro-1H-isoquinolin-2- |
| | yl]-cyclohexyl-methanone; |
| 39 | [6-(4-Cyclobutyl-[1,4]diazepane-1-carbonyl)-3,4-dihydro-1H-isoquinolin-2- |
| | yl]-cyclopentyl-methanone; |
| 40 | [6-(4-Cyclobutyl-[1,4]diazepane-1-carbonyl)-3,4-dihydro-1H-isoquinolin-2- |
| | yl]-cyclohexyl-methanone; |
| 41 | 1-[6-(4-Cyclobutyl-piperazine-1-carbonyl)-3,4-dihydro-1H-isoquinolin-2- |
| | yl]-2,2-dimethyl-propan-1-one; |
| 42 | (2-Chloro-phenyl)-[6-(4-cyclobutyl-piperazine-1-carbonyl)-3,4-dihydro-1H- |
| | isoquinolin-2-yl]-methanone; |
| 43 | 1-[6-(4-Cyclobutyl-piperazine-1-carbonyl)-3,4-dihydro-1H-isoquinolin-2- |
| | yl]-2-cyclopentyl-ethanone; |
| 44 | [6-(4-Cyclobutyl-piperazine-1-carbonyl)-3,4-dihydro-1H-isoquinolin-2-yl]- |
| | furan-3-yl-methanone; |
| 45 | (S)-1-[6-(2-Pyrrolidin-1-ylmethyl-pyrrolidine-1-carbonyl)-3,4-dihydro-1H- |
| | isoquinolin-2-yl]-propan-1-one; |
| 46 | (S)-1-[6-(2-Pyrrolidin-1-ylmethyl-pyrrolidine-1-carbonyl)-3,4-dihydro-1H- |
| | isoquinolin-2-yl]-butan-1-one; |
| 47 | (S)-2,2-Dimethyl-1-[6-(2-pyrrolidin-1-ylmethyl-pyrrolidine-1-carbonyl)-3,4- |
| | dihydro-1H-isoquinolin-2-yl]-propan-1-one; |
| 48 | (S)-Phenyl-[6-(2-pyrrolidin-1-ylmethyl-pyrrolidine-1-carbonyl)-3,4-dihydro- |
| | 1H-isoquinolin-2-yl]-methanone; |
| 49 | (S)-(4-tert-Butyl-phenyl)-[6-(2-pyrrolidin-1-ylmethyl-pyrrolidine-1- |
| | carbonyl)-3,4-dihydro-1H-isoquinolin-2-yl]-methanone; |
| 50 | (S)-(2-Chloro-phenyl)-[6-(2-pyrrolidin-1-ylmethyl-pyrrolidine-1-carbonyl)- |
| | 3,4-dihydro-1H-isoquinolin-2-yl]-methanone; |
| 51 | (S)-(3-Chloro-phenyl)-[6-(2-pyrrolidin-1-ylmethyl-pyrrolidine-1-carbonyl)- |
| | 3,4-dihydro-1H-isoquinolin-2-yl]-methanone; |
| 52 | (S)-3-[6-(2-Pyrrolidin-1-ylmethyl-pyrrolidine-1-carbonyl)-3,4-dihydro-1H- |
| | isoquinoline-2-carbonyl]-benzonitrile; |
| 53 | (S)-4-[6-(2-Pyrrolidin-1-ylmethyl-pyrrolidine-1-carbonyl)-3,4-dihydro-1H- |
| | isoquinoline-2-carbonyl]-benzonitrile; |
| 54 | (S)-[6-(2-Pyrrolidin-1-ylmethyl-pyrrolidine-1-carbonyl)-3,4-dihydro-1H- |
| | isoquinolin-2-yl]-o-tolyl-methanone; |
| 55 | (S)-[6-(2-Pyrrolidin-1-ylmethyl-pyrrolidine-1-carbonyl)-3,4-dihydro-1H- |
| | isoquinolin-2-yl]-p-tolyl-methanone; |
| 56 | (S)-(2-Fluoro-phenyl)-[6-(2-pyrrolidin-1-ylmethyl-pyrrolidine-1-carbonyl)- |
| | 3,4-dihydro-1H-isoquinolin-2-yl]-methanone; |
| 57 | (S)-(3-Fluoro-phenyl)-[6-(2-pyrrolidin-1-ylmethyl-pyrrolidine-1-carbonyl)- |
| | 3,4-dihydro-1H-isoquinolin-2-yl]-methanone; |
| 58 | (S)-(4-Fluoro-phenyl)-[6-(2-pyrrolidin-1-ylmethyl-pyrrolidine-1-carbonyl)- |
| | 3,4-dihydro-1H-isoquinolin-2-yl]-methanone; |
| 59 | (S)-(3-Methoxy-phenyl)-[6-(2-pyrrolidin-1-ylmethyl-pyrrolidine-1- |
| | carbonyl)-3,4-dihydro-1H-isoquinolin-2-yl]-methanone; |
| 60 | (S)-(4-Methoxy-phenyl)-[6-(2-pyrrolidin-1-ylmethyl-pyrrolidine-1- |
| | carbonyl)-3,4-dihydro-1H-isoquinolin-2-yl]-methanone; |
| 61 | (S)-2-Phenyl-1-[6-(2-pyrrolidin-1-ylmethyl-pyrrolidine-1-carbonyl)-3,4- |
| | dihydro-1H-isoquinolin-2-yl]-ethanone; |
| 62 | (4-Cyclobutyl-piperazin-1-yl)-[2-(3-fluoro-benzoyl)-1,2,3,4-tetrahydro- |
| | isoquinolin-6-yl]-methanone; |
| 63 | 1-[6-(4-Cyclopentyl-piperazine-1-carbonyl)-3,4-dihydro-1H-isoquinolin-2- |
| | yl]-propan-1-one; |
| 64 | 1-[6-(4-Cyclopentyl-piperazine-1-carbonyl)-3,4-dihydro-1H-isoquinolin-2- |
| | yl]-2,2-dimethyl-propan-1-one; |
| 65 | (2-Benzoyl-1,2,3,4-tetrahydro-isoquinolin-6-yl)-(4-cyclopentyl-piperazin-1- |
| | yl)-methanone; |
| 66 | (4-Cyclopentyl-piperazin-1-yl)-[2-(2-fluoro-benzoyl)-1,2,3,4-tetrahydro- |
| | isoquinolin-6-yl]-methanone; |
| 67 | (4-Cyclopentyl-piperazin-1-yl)-[2-(4-fluoro-benzoyl)-1,2,3,4-tetrahydro- |
| | isoquinolin-6-yl]-methanone; |
| 68 | [2-(2-Chloro-benzoyl)-1,2,3,4-tetrahydro-isoquinolin-6-yl]-(4-cyclopentyl- |
| | piperazin-1-yl)-methanone; |
| 69 | [2-(4-Chloro-benzoyl)-1,2,3,4-tetrahydro-isoquinolin-6-yl]-(4-cyclopentyl- |
| | piperazin-1-yl)-methanone; |
| 70 | (4-Cyclopentyl-piperazin-1-yl)-[2-(4-methyl-benzoyl)-1,2,3,4-tetrahydro- |
| | isoquinolin-6-yl]-methanone; |
| 71 | 1-[6-(4-Cyclopentyl-piperazine-1-carbonyl)-3,4-dihydro-1H-isoquinolin-2- |
| | yl]-2-(4-fluoro-phenyl)-ethanone; |
| 72 | (4-Cyclohexyl-piperazin-1-yl)-[2-(2-fluoro-benzoyl)-1,2,3,4-tetrahydro- |
| | isoquinolin-6-yl]-methanone; |
| 73 | (4-Cyclohexyl-piperazin-1-yl)-[2-(3-fluoro-benzoyl)-1,2,3,4-tetrahydro- |
| | isoquinolin-6-yl]-methanone; |
| 74 | (4-Cyclohexyl-piperazin-1-yl)-[2-(4-fluoro-benzoyl)-1,2,3,4-tetrahydro- |
| | isoquinolin-6-yl]-methanone; |
| 75 | [2-(2-Chloro-benzoyl)-1,2,3,4-tetrahydro-isoquinolin-6-yl]-(4-cyclohexyl- |
| | piperazin-1-yl)-methanone; |
| 76 | [2-(3-Chloro-benzoyl)-1,2,3,4-tetrahydro-isoquinolin-6-yl]-(4-cyclohexyl- |
| | piperazin-1-yl)-methanone; |
| 77 | [2-(4-Chloro-benzoyl)-1,2,3,4-tetrahydro-isoquinolin-6-yl]-(4-cyclohexyl- |
| | piperazin-1-yl)-methanone; |
| 78 | (4-Cyclohexyl-piperazin-1-yl)-[2-(2-methoxy-benzoyl)-1,2,3,4-tetrahydro- |
| | isoquinolin-6-yl]-methanone; |
| 79 | (4-Cyclohexyl-piperazin-1-yl)-[2-(3-methoxy-benzoyl)-1,2,3,4-tetrahydro- |
| | isoquinolin-6-yl]-methanone; |
| 80 | (3-[6-(4-Cyclohexyl-piperazine-1-carbonyl)-3,4-dihydro-1H-isoquinoline- |
| | 2-carbonyl]-benzonitrile; |
| 81 | 4-[6-(4-Cyclohexyl-piperazine-1-carbonyl)-3,4-dihydro-1H-isoquinoline-2- |
| | carbonyl]-benzonitrile; |
| 82 | (4-Cyclohexyl-piperazin-1-yl)-[2-(2-methyl-benzoyl)-1,2,3,4-tetrahydro- |
| | isoquinolin-6-yl]-methanone; |
| 83 | (4-Cyclohexyl-piperazin-1-yl)-[2-(4-methyl-benzoyl)-1,2,3,4-tetrahydro- |
| | isoquinolin-6-yl]-methanone; |
| 84 | [2-(4-tert-Butyl-benzoyl)-1,2,3,4-tetrahydro-isoquinolin-6-yl]-(4- |
| | cyclohexyl-piperazin-1-yl)-methanone; |
| 85 | (2-Benzoyl-1,2,3,4-tetrahydro-isoquinolin-6-yl)-(4-cyclohexyl-piperazin-1- |
| | yl)-methanone; |
| 86 | [2-(2-Fluoro-benzoyl)-1,2,3,4-tetrahydro-isoquinolin-6-yl]-(octahydro- |
| | pyrido[1,2-a]pyrazin-2-yl)-methanone; |
| 87 | [2-(3-Fluoro-benzoyl)-1,2,3,4-tetrahydro-isoquinolin-6-yl]-(octahydro- |
| | pyrido[1,2-a]pyrazin-2-yl)-methanone; |
| 88 | [2-(4-Fluoro-benzoyl)-1,2,3,4-tetrahydro-isoquinolin-6-yl]-(octahydro- |
| | pyrido[1,2-a]pyrazin-2-yl)-methanone; |
| 89 | [2-(2-Chloro-benzoyl)-1,2,3,4-tetrahydro-isoquinolin-6-yl]-(octahydro- |
| | pyrido[1,2-a]pyrazin-2-yl)-methanone; |
| 90 | [2-(3-Chloro-benzoyl)-1,2,3,4-tetrahydro-isoquinolin-6-yl]-(octahydro- |
| | pyrido[1,2-a]pyrazin-2-yl)-methanone; |
| 91 | [2-(4-Chloro-benzoyl)-1,2,3,4-tetrahydro-isoquinolin-6-yl]-(octahydro- |
| | pyrido[1,2-a]pyrazin-2-yl)-methanone; |
| 92 | [2-(2-Methoxy-benzoyl)-1,2,3,4-tetrahydro-isoquinolin-6-yl]-(octahydro- |
| | pyrido[1,2-a]pyrazin-2-yl)-methanone; |
| 93 | [2-(3-Methoxy-benzoyl)-1,2,3,4-tetrahydro-isoquinolin-6-yl]-(octahydro- |
| | pyrido[1,2-a]pyrazin-2-yl)-methanone; |
| 94 | 3-[6-(Octahydro-pyrido[1,2-a]pyrazine-2-carbonyl)-3,4-dihydro-1H- |
| | isoquinoline-2-carbonyl]-benzonitrile; |
| 95 | 4-[6-(Octahydro-pyrido[1,2-a]pyrazine-2-carbonyl)-3,4-dihydro-1H- |
| | isoquinoline-2-carbonyl]-benzonitrile; |
| 96 | [2-(2-Methyl-benzoyl)-1,2,3,4-tetrahydro-isoquinolin-6-yl]-(octahydro- |
| | pyrido[1,2-a]pyrazin-2-yl)-methanone; |
| 97 | [2-(4-Methyl-benzoyl)-1,2,3,4-tetrahydro-isoquinolin-6-yl]-(octahydro- |
| | pyrido[1,2-a]pyrazin-2-yl)-methanone; |
| 98 | [2-(4-tert-Butyl-benzoyl)-1,2,3,4-tetrahydro-isoquinolin-6-yl]-(octahydro- |
| | pyrido[1,2-a]pyrazin-2-yl)-methanone; |
| 99 | (2-Benzoyl-1,2,3,4-tetrahydro-isoquinolin-6-yl)-(octahydro-pyrido[1,2- |
| | a]pyrazin-2-yl)-methanone; |
| 100 | (4-Cyclobutyl-piperazin-1-yl)-(2-ethanesulfonyl-1,2,3,4-tetrahydro- |
| | isoquinolin-6-yl)-methanone; |
| 101 | (4-Cyclobutyl-piperazin-1-yl)-[2-(propane-1-sulfonyl)-1,2,3,4-tetrahydro- |
| | isoquinolin-6-yl]-methanone; |
| 102 | (4-Cyclobutyl-piperazin-1-yl)-[2-(propane-2-sulfonyl)-1,2,3,4-tetrahydro- |
| | isoquinolin-6-yl]-methanone; |
| 103 | (2-Benzenesulfonyl-1,2,3,4-tetrahydro-isoquinolin-6-yl)-(4-cyclobutyl- |
| | piperazin-1-yl)-methanone; |
| 104 | (4-Cyclobutyl-piperazin-1-yl)-[2-(4-fluoro-benzenesulfonyl)-1,2,3,4- |
| | tetrahydro-isoquinolin-6-yl]-methanone; |
| 105 | [2-(4-Chloro-benzoyl)-1,2,3,4-tetrahydro-isoquinolin-6-yl]-(4-cyclobutyl- |
| | piperazin-1-yl)-methanone; |
| 106 | (4-Isopropyl-piperazin-1-yl)-[2-(thiophene-3-carbonyl)-1,2,3,4-tetrahydro- |
| | isoquinolin-6-yl]-methanone; |
| 107 | [2-(4-Hydroxy-benzoyl)-1,2,3,4-tetrahydro-isoquinolin-6-yl]-(4-isopropyl- |
| | piperazin-1-yl)-methanone; |
| 108 | (4-Isopropyl-piperazin-1-yl)-[2-(4-methoxy-benzoyl)-1,2,3,4-tetrahydro- |
| | isoquinolin-6-yl]-methanone; |
| 109 | (4-Isopropyl-piperazin-1-yl)-[2-(4-methyl-benzoyl)-1,2,3,4-tetrahydro- |
| | isoquinolin-6-yl]-methanone; |
| 110 | [2-(4-Chloro-benzoyl)-1,2,3,4-tetrahydro-isoquinolin-6-yl]-(4-isopropyl- |
| | piperazin-1-yl)-methanone; |
| 111 | [2-(3,4-Dichloro-benzoyl)-1,2,3,4-tetrahydro-isoquinolin-6-yl]-(4-isopropyl- |
| | piperazin-1-yl)-methanone; |
| 112 | (4-Cyclobutyl-piperazin-1-yl)-[2-(4-methoxy-benzoyl)-1,2,3,4-tetrahydro- |
| | isoquinolin-6-yl]-methanone; |
| 113 | (4-Cyclobutyl-piperazin-1-yl)-[2-(4-methyl-benzoyl)-1,2,3,4-tetrahydro- |
| | isoquinolin-6-yl]-methanone; |
| 114 | (4-Cyclobutyl-piperazin-1-yl)-[2-(3,4-dichloro-benzoyl)-1,2,3,4-tetrahydro- |
| | isoquinolin-6-yl]-methanone; |
| 115 | (4-Cyclobutyl-piperazin-1-yl)-[2-(thiophene-3-carbonyl)-1,2,3,4- |
| | tetrahydro-isoquinolin-6-yl]-methanone; |
| 116 | [6-(4-Cyclobutyl-piperazine-1-carbonyl)-3,4-dihydro-1H-isoquinolin-2-yl]- |
| | (3-dimethylamino-phenyl)-methanone; |
| 117 | [6-(4-Cyclobutyl-piperazine-1-carbonyl)-3,4-dihydro-1H-isoquinolin-2-yl]- |
| | (4-dimethylamino-phenyl)-methanone; |
| 118 | [6-(4-Cyclobutyl-piperazine-1-carbonyl)-3,4-dihydro-1H-isoquinolin-2-yl]- |
| | (2,4-dichloro-phenyl)-methanone; |
| 119 | (3-Chloro-phenyl)-[6-(4-cyclobutyl-piperazine-1-carbonyl)-3,4-dihydro-1H- |
| | isoquinolin-2-yl]-methanone; |
| 120 | [6-(4-Cyclobutyl-piperazine-1-carbonyl)-3,4-dihydro-1H-isoquinolin-2-yl]- |
| | m-tolyl-methanone; |
| 121 | [6-(4-Cyclobutyl-piperazine-1-carbonyl)-3,4-dihydro-1H-isoquinolin-2-yl]- |
| | (3-nitro-phenyl)-methanone; |
| 122 | [6-(4-Cyclobutyl-piperazine-1-carbonyl)-3,4-dihydro-1H-isoquinolin-2-yl]- |
| | (4-nitro-phenyl)-methanone; |
| 123 | [6-(4-Cyclobutyl-piperazine-1-carbonyl)-3,4-dihydro-1H-isoquinolin-2-yl]- |
| | (4-hydroxy-phenyl)-methanone; |
| 124 | (4-Cyclobutyl-piperazin-1-yl)-[2-(4-fluoro-3-hydroxy-benzoyl)-1,2,3,4- |
| | tetrahydro-isoquinolin-6-yl]-methanone; |
| 125 | (4-Cyclobutyl-piperazin-1-yl)-[2-(4-fluoro-benzoyl)-1,2,3,4-tetrahydro- |
| | isoquinolin-6-yl]-methanone; |
| 126 | (4-Cyclobutyl-piperazin-1-yl)-[2-(2,4-difluoro-benzoyl)-1,2,3,4-tetrahydro- |
| | isoquinolin-6-yl]-methanone; |
| 127 | (4-Cyclobutyl-piperazin-1-yl)-[2-(3-fluoro-4-methyl-benzoyl)-1,2,3,4- |
| | tetrahydro-isoquinolin-6-yl]-methanone; |
| 128 | [2-(3-Chloro-4-fluoro-benzoyl)-1,2,3,4-tetrahydro-isoquinolin-6-yl]-(4- |
| | cyclobutyl-piperazin-1-yl)-methanone; |
| 129 | 1-[6-(4-Cyclobutyl-piperazine-1-carbonyl)-3,4-dihydro-1H-isoquinolin-2- |
| | yl]-2-phenyl-ethanone; |
| 130 | 1-[6-(4-Cyclobutyl-piperazine-1-carbonyl)-3,4-dihydro-1H-isoquinolin-2- |
| | yl]-2-(4-fluoro-phenyl)-ethanone; |
| 131 | [2-(4-tert-Butyl-benzoyl)-1,2,3,4-tetrahydro-isoquinolin-6-yl]-(4-cyclobutyl- |
| | piperazin-1-yl)-methanone; |
| 132 | (4-Cyclobutyl-piperazin-1-yl)-[2-(4-cyclohexyl-benzoyl)-1,2,3,4- |
| | tetrahydro-isoquinolin-6-yl]-methanone; |
| 133 | (4-Chloro-phenyl)-[6-(4-cyclobutyl-[1,4]diazepane-1-carbonyl)-3,4- |
| | dihydro-1H-isoquinolin-2-yl]-methanone; |
| 134 | [6-(4-Cyclobutyl-[1,4]diazepane-1-carbonyl)-3,4-dihydro-1H-isoquinolin-2- |
| | yl]-(4-fluoro-phenyl)-methanone; |
| 135 | (3-Chloro-phenyl)-[6-(4-cyclobutyl-[1,4]diazepane-1-carbonyl)-3,4- |
| | dihydro-1H-isoquinolin-2-yl]-methanone; |
| 136 | [6-(4-Cyclobutyl-[1,4]diazepane-1-carbonyl)-3,4-dihydro-1H-isoquinolin-2- |
| | yl]-(2-fluoro-phenyl)-methanone; |
| 137 | [6-(4-Cyclobutyl-[1,4]diazepane-1-carbonyl)-3,4-dihydro-1H-isoquinolin-2- |
| | yl]-(tetrahydro-furan-3-yl)-methanone; |
| 138 | [6-(4-Cyclobutyl-[1,4]diazepane-1-carbonyl)-3,4-dihydro-1H-isoquinolin-2- |
| | yl]-(tetrahydro-furan-2-yl)-methanone; |
| 139 | 1-[6-(4-Cyclobutyl-[1,4]diazepane-1-carbonyl)-3,4-dihydro-1H-isoquinolin- |
| | 2-yl]-propan-1-one; |
| 140 | [6-(4-Cyclobutyl-[1,4]diazepane-1-carbonyl)-3,4-dihydro-1H-isoquinolin-2- |
| | yl]-(4-propyl-phenyl)-methanone; |
| 141 | [6-(4-Cyclobutyl-[1,4]diazepane-1-carbonyl)-3,4-dihydro-1H-isoquinolin-2- |
| | yl]-(4-fluoro-3-hydroxy-phenyl)-methanone; |
| 142 | [6-(4-Cyclobutyl-[1,4]diazepane-1-carbonyl)-3,4-dihydro-1H-isoquinolin-2- |
| | yl]-(3-fluoro-4-methyl-phenyl)-methanone; |
| 143 | [6-(4-Cyclobutyl-[1,4]diazepane-1-carbonyl)-3,4-dihydro-1H-isoquinolin-2- |
| | yl]-(2,4-dichloro-phenyl)-methanone; |
| 144 | [6-(4-Cyclobutyl-[1,4]diazepane-1-carbonyl)-3,4-dihydro-1H-isoquinolin-2- |
| | yl]-(2,4-difluoro-phenyl)-methanone; |
| 145 | (3-Chloro-4-fluoro-phenyl)-[6-(4-cyclobutyl-[1,4]diazepane-1-carbonyl)- |
| | 3,4-dihydro-1H-isoquinolin-2-yl]-methanone; |
| 146 | [6-(4-Cyclobutyl-[1,4]diazepane-1-carbonyl)-3,4-dihydro-1H-isoquinolin-2- |
| | yl]-(3-methoxy-cyclohexyl)-methanone; |
| 147 | trans-[6-(4-Cyclobutyl-[1,4]diazepane-1-carbonyl)-3,4-dihydro-1H- |
| | isoquinolin-2-yl]-(4-methoxy-cyclohexyl)-methanone; |
| 148 | cis-[6-(4-Cyclobutyl-[1,4]diazepane-1-carbonyl)-3,4-dihydro-1H- |
| | isoquinolin-2-yl]-(4-methoxy-cyclohexyl)-methanone; |
| 149 | [2-(1-Isopropyl-piperidine-4-carbonyl)-1,2,3,4-tetrahydro-isoquinolin-6-yl]- |
| | morpholin-4-yl-methanone; |
| 150 | (S)-Cyclohexyl-[6-(2-pyrrolidin-1-ylmethyl-pyrrolidine-1-carbonyl)-3,4- |
| | dihydro-1H-isoquinolin-2-yl]-methanone; |
| 151 | Cyclohexyl-{6-[4-(tetrahydro-furan-2-ylmethyl)-piperazine-1-carbonyl]- |
| | 3,4-dihydro-1H-isoquinolin-2-yl}-methanone; |
| 152 | Cyclohexyl-[6-(octahydro-pyrido[1,2-a]pyrazine-2-carbonyl)-3,4-dihydro- |
| | 1H-isoquinolin-2-yl]-methanone; |
| 153 | Cyclohexyl-[6-(hexahydro-pyrrolo[1,2-a]pyrazine-2-carbonyl)-3,4-dihydro- |
| | 1H-isoquinolin-2-yl]-methanone; |
| 154 | Cyclohexyl-[6-(4-dimethylamino-piperidine-1-carbonyl)-3,4-dihydro-1H- |
| | isoquinolin-2-yl]-methanone; |
| 155 | (R)-Cyclohexyl-[6-(3-dimethylamino-pyrrolidine-1-carbonyl)-3,4-dihydro- |
| | 1H-isoquinolin-2-yl]-methanone; |
| 156 | (S)-Cyclohexyl-[6-(3-dimethylamino-pyrrolidine-1-carbonyl)-3,4-dihydro- |
| | 1H-isoquinolin-2-yl]-methanone; |
| 157 | [6-([1,4′]Bipiperidinyl-1′-carbonyl)-3,4-dihydro-1H-isoquinolin-2-yl]- |
| | cyclohexyl-methanone; |
| 158 | Cyclohexyl-[6-(4-morpholin-4-yl-piperidine-1-carbonyl)-3,4-dihydro-1H- |
| | isoquinolin-2-yl]-methanone; |
| 159 | Cyclohexyl-[6-(4-cyclopentyl-piperazine-1-carbonyl)-3,4-dihydro-1H- |
| | isoquinolin-2-yl]-methanone; |
| 160 | Cyclohexyl-[6-(4-cyclohexyl-piperazine-1-carbonyl)-3,4-dihydro-1H- |
| | isoquinolin-2-yl]-methanone; |
| 161 | 2-Cyclohexanecarbonyl-1,2,3,4-tetrahydro-isoquinoline-6-carboxylic acid |
| | methyl-(1-methyl-pyrrolidin-3-yl)-amide; |
| 162 | Cyclohexyl-[6-(4-isopropyl-[1,4]diazepane-1-carbonyl)-3,4-dihydro-1H- |
| | isoquinolin-2-yl]-methanone; |
| 163 | (5-Cyclobutyl-hexahydro-pyrrolo[3,4-c]pyrrol-2-yl)-(2- |
| | cyclohexanecarbonyl-1,2,3,4-tetrahydro-isoquinolin-6-yl)-methanone; |
| 164 | (1S,4S)-(5-Cyclobutyl-2,5-diaza-bicyclo[2.2.1]hept-2-yl)-(2- |
| | cyclohexanecarbonyl-1,2,3,4-tetrahydro-isoquinolin-6-yl)-methanone; |
| 165 | (1-Cyclobutyl-hexahydro-pyrrolo[3,4-b]pyrrol-5-yl)-(2- |
| | cyclohexanecarbonyl-1,2,3,4-tetrahydro-isoquinolin-6-yl)-methanone; |
| 166 | Cyclohexyl-(6-piperidin-1-ylmethyl-3,4-dihydro-1H-isoquinolin-2-yl)- |
| | methanone; |
| 167 | Cyclohexyl-(6-morpholin-4-ylmethyl-3,4-dihydro-1H-isoquinolin-2-yl)- |
| | methanone; |
| 168 | Cyclohexyl-[6-(octahydro-pyrido[1,2-a]pyrazin-2-ylmethyl)-3,4-dihydro- |
| | 1H-isoquinolin-2-yl]-methanone; |
| 169 | Cyclohexyl-[6-(2-pyrrolidin-1-ylmethyl-pyrrolidin-1-ylmethyl)-3,4-dihydro- |
| | 1H-isoquinolin-2-yl]-methanone; |
| 170 | [6-(4-Cyclobutyl-piperazin-1-ylmethyl)-3,4-dihydro-1H-isoquinolin-2-yl]- |
| | cyclohexyl-methanone; |
| 171 | [6-(4-Cyclobutyl-[1,4]diazepan-1-ylmethyl)-3,4-dihydro-1H-isoquinolin-2- |
| | yl]-cyclohexyl-methanone; |
| 172 | (2-Cyclopentyl-1,2,3,4-tetrahydro-isoquinolin-6-yl)-morpholin-4-yl- |
| | methanone; |
| 173 | (2-Isopropyl-1,2,3,4-tetrahydro-isoquinolin-6-yl)-piperidin-1-yl- |
| | methanone; |
| 174 | (2-Isopropyl-1,2,3,4-tetrahydro-isoquinolin-6-yl)-morpholin-4-yl- |
| | methanone; |
| 175 | (2-Isopropyl-1,2,3,4-tetrahydro-isoquinolin-6-yl)-(octahydro-pyrido[1,2- |
| | a]pyrazin-2-yl)-methanone; |
| 176 | (4-tert-Butyl-piperidin-1-yl)-(2-isopropyl-1,2,3,4-tetrahydro-isoquinolin-6- |
| | yl)-methanone; |
| 177 | (4-Cyclobutyl-[1,4]diazepan-1-yl)-(2-isopropyl-1,2,3,4-tetrahydro- |
| | isoquinolin-6-yl)-methanone; |
| 178 | [4-(1-Hydroxy-1-methyl-ethyl)-piperidin-1-yl]-(2-isopropyl-1,2,3,4- |
| | tetrahydro-isoquinolin-6-yl)-methanone; |
| 179 | Piperidin-1-yl-(2-propyl-1,2,3,4-tetrahydro-isoquinolin-6-yl)-methanone; |
| 180 | Morpholin-4-yl-(2-propyl-1,2,3,4-tetrahydro-isoquinolin-6-yl)-methanone; |
| 181 | (Octahydro-pyrido[1,2-a]pyrazin-2-yl)-(2-propyl-1,2,3,4-tetrahydro- |
| | isoquinolin-6-yl)-methanone; |
| 182 | (4-tert-Butyl-piperidin-1-yl)-(2-propyl-1,2,3,4-tetrahydro-isoquinolin-6-yl)- |
| | methanone; |
| 183 | (4-Cyclobutyl-[1,4]diazepan-1-yl)-(2-propyl-1,2,3,4-tetrahydro-isoquinolin- |
| | 6-yl)-methanone; |
| 184 | [4-(1-Hydroxy-1-methyl-ethyl)-piperidin-1-yl]-(2-propyl-1,2,3,4-tetrahydro- |
| | isoquinolin-6-yl)-methanone; |
| 185 | (2-Cyclobutyl-1,2,3,4-tetrahydro-isoquinolin-6-yl)-piperidin-1-yl-methanone; |
| 186 | (2-Cyclobutyl-1,2,3,4-tetrahydro-isoquinolin-6-yl)-morpholin-4-yl-methanone; |
| 187 | (2-Cyclobutyl-1,2,3,4-tetrahydro-isoquinolin-6-yl)-(octahydro-pyrido[1,2- |
| | a]pyrazin-2-yl)-methanone; |
| 188 | (4-tert-Butyl-piperidin-1-yl)-(2-cyclobutyl-1,2,3,4-tetrahydro-isoquinolin-6- |
| | yl)-methanone; |
| 189 | (4-Cyclobutyl-[1,4]diazepan-1-yl)-(2-cyclobutyl-1,2,3,4-tetrahydro- |
| | isoquinolin-6-yl)-methanone; |
| 190 | (2-Cyclobutyl-1,2,3,4-tetrahydro-isoquinolin-6-yl)-[4-(1-hydroxy-1-methyl- |
| | ethyl)-piperidin-1-yl]-methanone; |
| 191 | (2-Cyclopentyl-1,2,3,4-tetrahydro-isoquinolin-6-yl)-piperidin-1-yl- |
| | methanone; |
| 192 | (2-Cyclopentyl-1,2,3,4-tetrahydro-isoquinolin-6-yl)-(octahydro-pyrido[1,2- |
| | a]pyrazin-2-yl)-methanone; |
| 193 | (4-tert-Butyl-piperidin-1-yl)-(2-cyclopentyl-1,2,3,4-tetrahydro-isoquinolin- |
| | 6-yl)-methanone; and |
| 194 | (2-Cyclopentyl-1,2,3,4-tetrahydro-isoquinolin-6-yl)-[4-(1-hydroxy-1- |
| | methyl-ethyl)-piperidin-1-yl]-methanone; |

and pharmaceutically acceptable salts thereof.

The invention includes also pharmaceutically acceptable salts of the compounds of Formula (I), preferably of those described above and of the specific compounds exemplified herein, and methods of treatment using such salts.

A “pharmaceutically acceptable salt” is intended to mean a salt of a free acid or base of a compound represented by Formula (I) that is non-toxic, biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Examples of pharmaceutically acceptable salts are those that are pharmacologically effective and suitable for contact with the tissues of patients without undue toxicity, irritation, or allergic response. A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound of Formula (I) contains a basic nitrogen, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents or acceptable substitutes in light of the ordinary level of skill in this technology.

If the compound of Formula (I) is an acid, such as a carboxylic acid or sulfonic acid, the desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, and piperazine, and inorganic salts derived from sodium, calcium, potassium, magnesium, manganese, iron, copper, zinc, aluminum, and lithium.

The invention also relates to pharmaceutically acceptable prodrugs of the compounds of Formula (I), and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

Examples of prodrugs include compounds having an amino acid residue, or a polypeptide chain of two or more (e.g., two, three or four) amino acid residues, covalently joined through an amide or ester bond to a free amino, hydroxy, or carboxylic acid group of a compound of Formula (I). Examples of amino acid residues include the twenty naturally occurring amino acids, commonly designated by three letter symbols, as well as 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Additional types of prodrugs may be produced, for instance, by derivatizing free carboxyl groups of structures of Formula (I) as amides or alkyl esters. Examples of amides include those derived from ammonia, primary C₁₋₆alkyl amines and secondary di(C₁₋₆alkyl) amines. Secondary amines include 5- or 6-membered heterocycloalkyl or heteroaryl ring moieties. Examples of amides include those that are derived from ammonia, C₁₋₃alkyl primary amines, and di(C₁₋₂alkyl)amines. Examples of esters of the invention include C₁₋₇alkyl, C₅₋₇cycloalkyl, phenyl, and phenyl(C₁₋₆alkyl) esters. Preferred esters include methyl esters. Prodrugs may also be prepared by derivatizing free hydroxy groups using groups including hemisuccinates, phosphate esters, dimethylaminoacetates, and phosphoryloxymethyloxycarbonyls, following procedures such as those outlined in Adv. Drug Delivery Rev. 1996, 19, 115. Carbamate derivatives of hydroxy and amino groups may also yield prodrugs. Carbonate derivatives, sulfonate esters, and sulfate esters of hydroxy groups may also provide prodrugs. Derivatization of hydroxy groups as (acyloxy)methyl and (acyloxy)ethyl ethers, wherein the acyl group may be an alkyl ester, optionally substituted with one or more ether, amine, or carboxylic acid functionalities, or where the acyl group is an amino acid ester as described above, is also useful to yield prodrugs. Prodrugs of this type may be prepared as described in J. Med. Chem. 1996, 39, 10. Free amines can also be derivatized as amides, sulfonamides or phosphonamides. All of these prodrug moieties may incorporate groups including ether, amine, and carboxylic acid functionalities.

The present invention also relates to pharmaceutically active metabolites of the compounds of Formula (I), which may also be used in the methods of the invention. A “pharmaceutically active metabolite” means a pharmacologically active product of metabolism in the body of a compound of Formula (I) or salt thereof. Prodrugs and active metabolites of a compound may be determined using routine techniques known or available in the art. See, e.g., Bertolini, et al., J. Med. Chem. 1997, 40, 2011-2016; Shan, et al., J. Pharm. Sci. 1997, 86 (7), 765-767; Bagshawe, Drug Dev. Res. 1995, 34, 220-230; Bodor, Adv. Drug Res. 1984, 13, 224-331; Bundgaard, Design of Prodrugs (Elsevier Press, 1985); and Larsen, Design and Application of Prodrugs, Drug Design and Development (Krogsgaard-Larsen, et al., eds., Harwood Academic Publishers, 1991).

The compounds of Formula (I) and their pharmaceutically acceptable salts, pharmaceutically acceptable prodrugs, and pharmaceutically active metabolites of the present invention are useful as modulators of the histamine H₃ receptor in the methods of the invention. As such modulators, the compounds may act as antagonists, agonists, or inverse agonists. “Modulators” include both inhibitors and activators, where “inhibitors” refer to compounds that decrease, prevent, inactivate, desensitize or down-regulate histamine H₃ receptor expression or activity, and “activators” are compounds that increase, activate, facilitate, sensitize, or up-regulate histamine H₃ receptor expression or activity.

The term “treat” or “treating” as used herein is intended to refer to administration of an active agent or composition of the invention to a subject for the purpose of effecting a therapeutic or prophylactic benefit through modulation of histamine H₃ receptor activity. Treating includes reversing, ameliorating, alleviating, inhibiting the progress of, lessening the severity of, or preventing a disease, disorder, or condition, or one or more symptoms of such disease, disorder or condition mediated through modulation of histamine H₃ receptor activity. The term “subject” refers to a mammalian patient in need of such treatment, such as a human.

Accordingly, the invention relates to methods of using the compounds described herein to treat subjects diagnosed with or suffering from a disease, disorder, or condition mediated by histamine H₃ receptor activity, such as: cognitive disorders, sleep disorders, psychiatric disorders, and other disorders. Symptoms or disease states are intended to be included within the scope of “medical conditions, disorders, or diseases.”

Cognitive disorders include, for example, dementia, Alzheimer's disease (Panula, P. et al., Soc. Neurosci. Abstr. 1995, 21, 1977), cognitive dysfunction, mild cognitive impairment (pre-dementia), attention deficit hyperactivity disorders (ADHD), attention-deficit disorders, and learning and memory disorders (Barnes, J. C. et al., Soc. Neurosci. Abstr. 1993, 19, 1813). Learning and memory disorders include, for example, learning impairment, memory impairment, age-related cognitive decline, and memory loss. H₃ antagonists have been shown to improve memory in a variety of memory tests, including the elevated plus maze in mice (Miyazaki, S. et al. Life Sci. 1995, 57(23), 2137-2144), a two-trial place recognition task (Orsetti, M. et al. Behav. Brain Res. 2001, 124(2), 235-242), the passive avoidance test in mice (Miyazaki, S. et al. Meth. Find. Exp. Clin. Pharmacol. 1995, 17(10), 653-658) and the radial maze in rats (Chen, Z. Acta Pharmacol. Sin. 2000, 21(10), 905-910). Also, in the spontaneously hypertensive rat, an animal model for the learning impairments in attention-deficit disorders, H₃ antagonists were shown to improve memory (Fox, G. B. et al. Behav. Brain Res. 2002, 131(1-2), 151-161).

Sleep disorders include, for example, insomnia, disturbed sleep, narcolepsy (with or without associated cataplexy), cataplexy, disorders of sleep/wake homeostasis, idiopathic somnolence, excessive daytime sleepiness (EDS), circadian rhythm disorders, fatigue, lethargy, jet lag (phase delay), and REM-behavioral disorder. Fatigue and/or sleep impairment may be caused by or associated with various sources, such as, for example, sleep apnea, perimenopausal hormonal shifts, Parkinson's disease, multiple sclerosis (MS), depression, chemotherapy, or shift work schedules.

Psychiatric disorders include, for example, schizophrenia (Schlicker, E. and Marr, I., Naunyn-Schmiedeberg's Arch. Pharmacol. 1996, 353, 290-294), including cognitive deficits and negative symptoms associated with schizophrenia, bipolar disorders, manic disorders, depression (Lamberti, C. et al. Br. J. Pharmacol. 1998, 123(7), 1331-1336; Perez-Garcia, C. et al. Psychopharmacology 1999, 142(2), 215-220) (Also see: Stark, H. et al., Drugs Future 1996, 21(5), 507-520; and Leurs, R. et al., Prog. Drug Res. 1995, 45, 107-165 and references cited therein.), including bipolar depression, obsessive-compulsive disorder, and post-traumatic stress disorder.

Other disorders include, for example, motion sickness, vertigo (e.g. vertigo or benign postural vertigo), tinitus, epilepsy (Yokoyama, H. et al., Eur. J. Pharmacol. 1993, 234, 129-133), migraine, neurogenic inflammation, neuropathic pain, Down Syndrome, seizures, eating disorders (Machidori, H. et al., Brain Res. 1992, 590, 180-186), obesity, substance abuse disorders, movement disorders (e.g. restless legs syndrome), and eye-related disorders (e.g. macular degeneration and retinitis pigmentosis).

Particularly, as modulators of the histamine H₃ receptor, the compounds of the present invention are useful in the treatment or prevention of depression, disturbed sleep, narcolepsy, fatigue, lethargy, cognitive impairment, memory impairment, memory loss, learning impairment, attention-deficit disorders, and eating disorders.

In treatment methods according to the invention, an effective amount of at least one compound according to the invention is administered to a subject suffering from or diagnosed as having such a disease, disorder, or condition. An “effective amount” means an amount or dose sufficient to generally bring about the desired therapeutic or prophylactic benefit in patients in need of such treatment for the designated disease, disorder, or condition. Effective amounts or doses of the compounds of the present invention may be ascertained by routine methods such as modeling, dose escalation studies or clinical trials, and by taking into consideration routine factors, e.g., the mode or route of administration or drug delivery, the pharmacokinetics of the compound, the severity and course of the disease, disorder, or condition, the subject's previous or ongoing therapy, the subject's health status and response to drugs, and the judgment of the treating physician. An example of a dose is in the range of from about 0.001 to about 200 mg of compound per kg of subject's body weight per day, preferably about 0.05 to 100 mg/kg/day, or about 1 to 35 mg/kg/day, in single or divided dosage units (e.g., BID, TID, QID). For a 70-kg human, an illustrative range for a suitable dosage amount is from about 0.05 to about 7 g/day, or about 0.2 to about 2.5 g/day.

Once improvement of the patient's disease, disorder, or condition has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

In addition, the compounds of the invention may be used in combination with additional active ingredients in the treatment of the above conditions. In an exemplary embodiment, additional active ingredients are those that are known or discovered to be effective in the treatment of conditions, disorders, or diseases mediated by histamine H₃ receptor activity or that are active against another target associated with the particular condition, disorder, or disease, such as H₁ receptor antagonists, H₂ receptor antagonists, H₃ receptor antagonists, topiramate (TOPAMAX™), and neurotransmitter modulators such as serotonin-norepinephrine reuptake inhibitors, selective serotonin reuptake inhibitors (SSRIs), noradrenergic reuptake inhibitors, non-selective serotonin re-uptake inhibitors (NSSRIs), acetylcholinesterase inhibitors (such as tetrahydroaminoacridine, Donepezil (ARICEPT™), Rivastigmine, or Galantamine (REMINYL™)), or modafinil. The combination may serve to increase efficacy (e.g., by including in the combination a compound potentiating the potency or effectiveness of a compound according to the invention), decrease one or more side effects, or decrease the required dose of the compound according to the invention.

More particularly, compounds of the invention in combination with modafinil are useful for the treatment of narcolepsy, excessive daytime sleepiness (EDS), Alzheimer's disease, depression, attention-deficit disorders, MS-related fatigue, post-anesthesia grogginess, cognitive impairment, schizophrenia, spasticity associated with cerebral palsy, age-related memory decline, idiopathic somnolence, or jet-lag. Preferably, the combination method employs doses of modafinil in the range of about 20 to 300 mg per dose.

In another embodiment, compounds of the invention in combination with topiramate are useful for the treatment of obesity. Preferably, the combination method employs doses of topiramate in the range of about 20 to 300 mg per dose.

The compounds of the invention are used, alone or in combination with one or more other active ingredients, to formulate pharmaceutical compositions of the invention. A pharmaceutical composition of the invention comprises: (a) an effective amount of a compound of Formula (I), or a pharmaceutically acceptable salt, pharmaceutically acceptable prodrug, or pharmaceutically active metabolite thereof; and (b) a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of a compound of the invention and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the compounds of the invention may be prepared using suitable pharmaceutical excipients and compounding techniques now or later known or available to those skilled in the art. The compositions may be administered in the inventive methods by oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the compounds of the invention can be provided in the form of tablets or capsules, or as a solution, emulsion, or suspension. To prepare the oral compositions, the compounds may be formulated to yield a dosage of, e.g., from about 0.01 to about 100 mg/kg daily, or from about 0.05 to about 35 mg/kg daily, or from about 0.1 to about 10 mg/kg daily.

Oral tablets may include a compound according to the invention mixed with pharmaceutically acceptable excipients such as inert diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are suitable disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, compounds of the invention may be mixed with a solid, semi-solid, or liquid diluent. Soft gelatin capsules may be prepared by mixing the compound of the invention with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The compounds of this invention may also be administered by non-oral routes. For example, the compositions may be formulated for rectal administration as a suppository. For parenteral use, including intravenous, intramuscular, intraperitoneal, or subcutaneous routes, the compounds of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of compound, admixed with a pharmaceutical carrier over a period ranging from several minutes to several days.

For topical administration, the compounds may be mixed with a pharmaceutical carrier at a concentration of about 0.1% to about 10% of drug to vehicle. Another mode of administering the compounds of the invention may utilize a patch formulation to affect transdermal delivery.

Compounds of the invention may alternatively be administered in methods of this invention by inhalation, via the nasal or oral routes, e.g., in a spray formulation also containing a suitable carrier.

Exemplary compounds useful in methods of the invention will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I). Reactions may be performed between the melting point and the reflux temperature of the solvent, and preferably between 0° C. and the reflux temperature of the solvent.

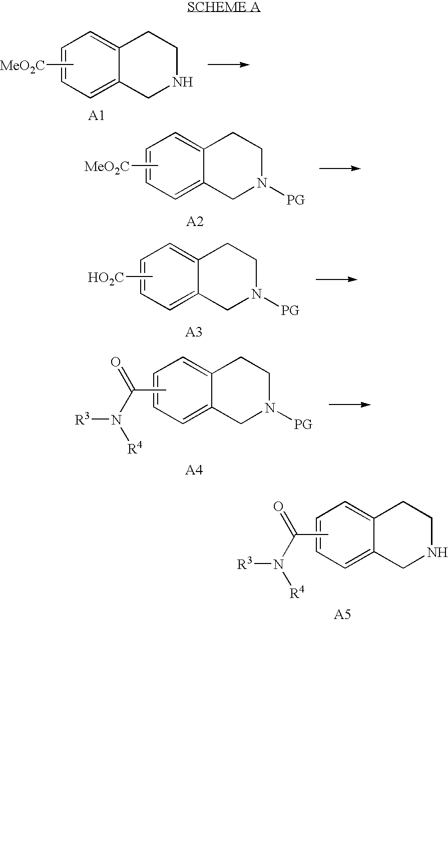

Certain embodiments of compounds of Formula (I), such as amides A5, are prepared from commercially available alkyl ester substituted tetrahydro-isoquinoline derivatives (such as A1) as shown in Scheme A. Installation of a suitable nitrogen protecting group under standard conditions gives protected amines A2. Preferably, PG is a tert-butoxycarbonyl group. Hydrolysis of the ester moiety under general conditions provides acids A3 or their corresponding salts. Coupling of acids A3 with suitable amines HNR³R⁴ gives amides A4. Preferred reaction conditions include, for example: 1) treatment with 1-(3-dimethylaminopropyl)-3-ethylcarbodiimide (EDC) and 1-hydroxybenzotriazole (HOBt) in a solvent such as N,N-dimethylformamide (DMF); or 2) formation of the mixed anhydride and subsequent treatment with amines HNR³R⁴. Removal of the PG protecting group under conditions known in the art provides amides A5.

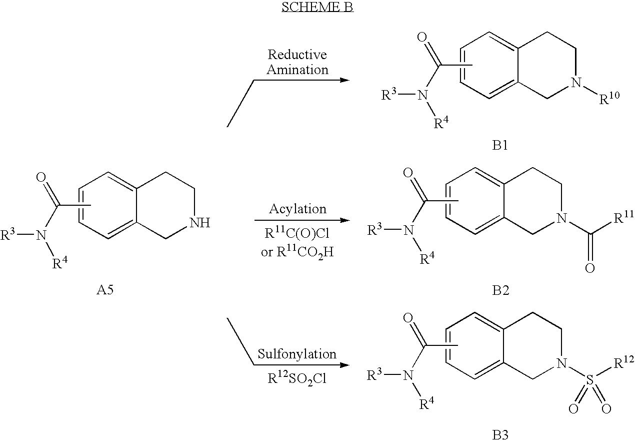

Further embodiments of compounds of Formula (I), such as compounds B1 (where R¹⁰ is C₁₋₄alkyl, C₃₋₇cycloalkyl, —CH₂-phenyl, or —CH₂-(monocyclic heteroaryl), B2 (where R¹¹ is —C₁₋₄alkyl, —C₃₋₇cycloalkyl, -(monocyclic heterocycloalkyl), -phenyl, -(monocyclic heteroaryl), —CH₂—C₃₋₇cycloalkyl, —CH₂-phenyl, or —CH₂-(monocyclic heteroaryl), and B3 (where R¹² is C₁₋₄alkyl or phenyl), are prepared as shown in Scheme B. Reductive amination of amines A5 with a suitable aldehyde or ketone provides amines B1. Preferred conditions include treatment with a reducing agent such as NaBH(OAc)₃ or NaCNBH₃ in a solvent such as 1,2-dichloroethane (DCE), with optional additives such as acetic acid or a Lewis acid (e.g. ZnCl₂). Formation of amides B2 is accomplished by, for example: 1) reacting amines A5 with acid chlorides R¹¹C(O)Cl in the presence of a suitable base such as triethylamine, in a solvent such as dichloromethane (DCM); 2) reacting amines A5 with acids R¹¹CO₂H under peptide coupling conditions; or 3) preparing the corresponding mixed anhydrides and reacting with R¹¹—OH. Synthesis of sulfonamides B3 is done by reacting amines A5 with sulfonyl chlorides R¹²SO₂Cl in the presence of a suitable base (such as triethylamine) in a solvent such as DCM.

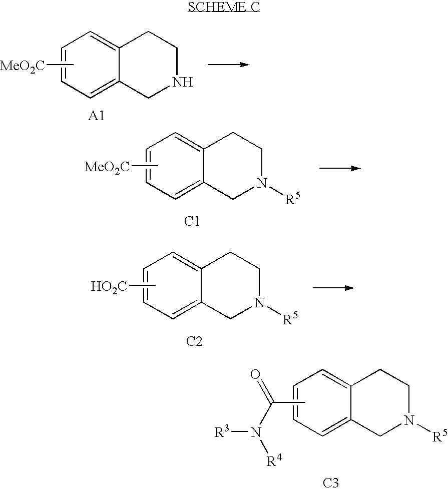

Further embodiments of compounds of Formula (I), such as compounds C3) are prepared according to Scheme C. Esters A1 are reacted using methods described in Scheme B to provide R⁵-substituted esters C1. Hydrolysis and amide formation as described in Scheme A give rise to compounds C3.

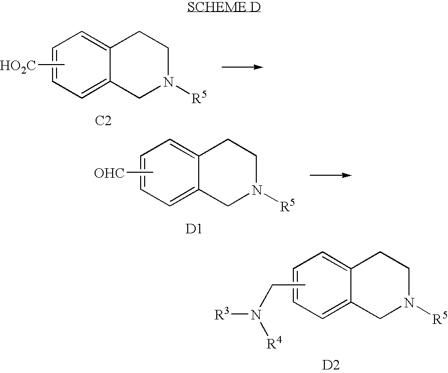

Further embodiments of Formula (I), such as amines D2, are prepared as shown in Scheme C. Acids C2 are reduced to aldehydes D1 using standard methods (such as, for example, reduction via a corresponding mixed anhydride or ester to the alcohol followed by oxidation to the aldehyde; or by conversion to an ester and subsequent reduction to the aldehyde). Aldehydes D1 are reacted with amines HNR³R⁴ under reductive amination conditions to provide aminomethyl compounds D2.

Those skilled in the art will recognize that several of the chemical transformations described above may be performed in a different order than that depicted in the above Schemes.

Compounds of Formula (I) may be converted to their corresponding salts using methods known to those skilled in the art. For example, amines of Formula (I) may be treated with trifluoroacetic acid (TFA), HCl, maleic acid, or citric acid in a solvent such as diethyl ether (Et₂O), DCM, tetrahydrofuran (THF), or methanol (MeOH) to provide the corresponding salt forms.

Compounds prepared according to the schemes described above may be obtained as single enantiomers, diastereomers, or regioisomers, by enantio-, diastero-, or regiospecific synthesis, or by resolution. Compounds prepared according to the schemes above may alternately be obtained as racemic (1:1) or non-racemic (not 1:1) mixtures or as mixtures of diastereomers or regioisomers. Where racemic and non-racemic mixtures of enantiomers are obtained, single enantiomers may be isolated using conventional separation methods known to one skilled in the art, such as chiral chromatography, recrystallization, diastereomeric salt formation, derivatization into diastereomeric adducts, biotransformation, or enzymatic transformation. Where regioisomeric or diastereomeric mixtures are obtained, single isomers may be separated using conventional methods such as chromatography or crystallization.

The following examples are provided to further illustrate the invention and various preferred embodiments.

### EXAMPLES

### Chemistry

In preparing the compounds described in the examples below and obtaining the corresponding analytical data, the following experimental and analytical protocols were followed unless otherwise indicated.

Unless otherwise specified, reaction mixtures were magnetically stirred at room temperature (rt) under a N_{2(g)} atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure.

Normal phase flash column chromatography (FCC) was typically performed with RediSep® silica gel columns using MeOH/DCM or 2 M NH₃ in MeOH/DCM as eluent, unless otherwise indicated.

Reverse phase high performance liquid chromatography (HPLC) was performed on a Dionex APS2000 LC/MS with a Phenomenex Gemini C18 (5 μm, 30×100 mm) column, and a gradient of 5 to 100% acetonitrile/water (20 mM NH₄OH) over 16.3 min, and a flow rate of 30 mL/min.

Mass spectra (MS) were obtained on an Agilent series 1100 MSD using electrospray ionization (ESI) in positive mode unless otherwise indicated. Calculated (calcd.) mass corresponds to the exact mass.

Nuclear magnetic resonance (NMR) spectra were obtained on Bruker model DRX spectrometers. The format of the ¹H NMR data below is: chemical shift in ppm downfield of the tetramethylsilane reference (multiplicity, coupling constant J in Hz, integration). For multiplicity, “p” indicates a quintuplet.

Chemical names were generated using ChemDraw Ultra 6.0.2 (CambridgeSoft Corp., Cambridge, Mass.).

### Example 1

### 6-(4-Isopropyl-[1,4]diazepane-1-carbonyl)-3,4-dihydro-1H-isoquinoline-2-carboxylic acid tert-butyl ester

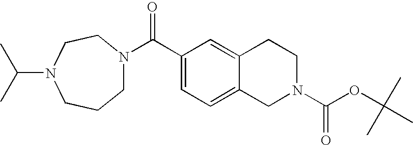

Step A: 1-Isopropyl-[1,4]diazepane. A solution of N-Boc-homopiperazine (20.0 g, 100 mmol), and acetone (7.4 mL, 100 mmol) in DCE (330 mL) was treated with NaBH(OAc)₃ (22.25 g, 105 mmol). After stirring overnight, the mixture was washed with 1 N NaOH (2×). The organic layer was dried and concentrated to provide 4-isopropyl-[1,4]diazepane-1-carboxylic acid tert-butyl ester as a pale yellow liquid. ¹H NMR (CDCl₃): 3.50-3.36 (m, 4H), 2.90 (dsept, J=6.6, 1.6, 1H), 2.67-2.53 (m, 4H), 1.85-1.49 (m, 2H), 1.46 (s, 9H), 1.00 (d, J=6.6, 3H), 0.99 (d, J=6.6, 3H). A rapidly stirring solution of crude 4-isopropyl-[1,4]diazepane-1-carboxylic acid tert-butyl ester in 1,4-dioxane (50 mL) was treated with HCl (4.0 M in 1,4-dioxane; 125 mL) at a moderate rate, producing a gummy precipitate. The mixture was heated at 45° C. for 6 h. The mixture was concentrated to provide the 1-isopropyl-[1,4]diazepane hydrochloride salt as a viscous liquid. The crude salt was dissolved in water (300 mL), basified with NaOH (250 g), and extracted with DCM. The combined organic layers were dried and concentrated to provide the free base of the title diazepane as a colorless liquid (11.7 g, 82% over 2 steps). ¹H NMR (CDCl₃): 2.97-2.85 (m, 5H), 2.70-2.62 (m, 4H), 2.25-2.08 (bm, 1H), 1.78-1.69 (m, 2H), 1.01 (d, J=6.6, 6H).

Step B: 3,4-Dihydro-1H-isoquinoline-2,6-dicarboxylic acid 2-tert-butyl ester 6-methyl ester. To a solution of 6-methoxycarbonyl-1,2,3,4-tetrahydroisoquinoline hydrochloride (5.00 g, 22.0 mmol) in MeOH (220 mL) was added di-tert-butyl dicarbonate (7.20 g, 33.0 mmol) and triethylamine (TEA; 9.20 mL, 66.0 mmol). After 24 h, the mixture was concentrated to provide a yellow oil. This oil was dissolved in ethyl acetate (EtOAc; 200 mL) and washed with 0.25 M HCl (200 mL). The aqueous layer was extracted with EtOAc. The combined organic layers were dried and concentrated to provide 6.84 g (100%) of the title compound as a colorless oil. The oil was used in the next step without further purification.

Step C: Potassium 2-tert-butoxycarbonyl-1,2,3,4-tetrahydro-isoquinoline-6-carboxylate. To a solution of 3,4-dihydro-1H-isoquinoline-2,6-dicarboxylic acid 2-tert-butyl ester 6-methyl ester (6.84 g, 23.5 mmol) in i-PrOH (220 mL) was added 2 N KOH (13.2 mL, 26.4 mmol). The solution was stirred at 80° C. for 24 h and then concentrated to provide 7.37 g (100%) of the title compound as a white solid. The solid was used in the next step without further purification.

Step D: 6-(4-Isopropyl-[1,4]diazepane-1-carbonyl)-3,4-dihydro-1H-isoquinoline-2-carboxylic acid tert-butyl ester. A solution of potassium 2-tert-butoxycarbonyl-1,2,3,4-tetrahydro-isoquinoline-6-carboxylate (1.00 g, 3.17 mmol) and EDC (0.913 g, 4.76 mmol) in DMF (30 mL) was stirred until the solution was clear and then was treated with HOBt (0.643 g, 4.76 mmol) and 1-isopropyl-[1,4]diazepane (0.900 g, 6.35 mmol). After 16 h, the reaction mixture was concentrated and the resulting residue was dissolved in DCM (30 mL) and washed with 1 N NaOH (30 mL). The aqueous layer was extracted with DCM (3×30 mL). The combined organic layers were washed with brine, dried and concentrated. The resulting yellow oil was purified by FCC to provide 1.13 g (89%) of the title compound as a white solid. MS (ESI): mass calcd. for C₂₃H₃₅N₃O₃, 401.27; m/z found, 402.3 [M+H]⁺. ¹H NMR (CDCl₃; mixture of rotamers): 7.19 (d, J=7.8, 1H), 7.18 (s, 1H), 7.11 (d, J=7.8, 1H), 4.58 (s, 2H) 3.76-3.74 (m, 2H), 3.65 (bs, 2H), 3.46-3.42 (m, 2H), 2.97-2.87 (m, 1H) 2.84 (t, J=5.2, 2H), 2.79 (t, J=4.7, 1H), 2.68 (t, J=5.7, 1H), 2.62-2.57 (m, 2H) 1.91 (p, J=5.7, 1H), 1.73 (p, J=4.7, 1H), 1.49 (s, 9H), 1.03 (d, J=6.6, 3H), 0.98 (d, J=6.6, 3H).

The compounds in Examples 2-4 were prepared using methods analogous to those described for Example 1, Steps B-D.

### Example 2

### 6-(4-Cyclopentyl-piperazine-1-carbonyl)-3,4-dihydro-1H-isoquinoline-2-carboxylic acid tert-butyl ester

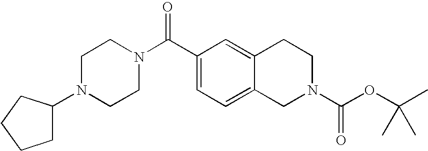

MS (ESI): mass calcd. for C₂₄H₃₅N₃O₂, 413.56; m/z found, 414.3 [M+H]⁺.

### Example 3

### 6-(4-Cyclohexyl-piperazine-1-carbonyl)-3,4-dihydro-1H-isoquinoline-2-carboxylic acid tert-butyl ester

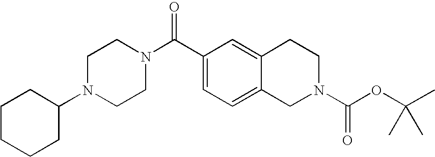

MS (ESI): mass calcd. for C₂₅H₃₇N₃O₃, 427.59; m/z found, 428.3 [M+H]⁺.

### Example 4

### 6-(Octahydro-pyrido[1,2-a]pyrazine-2-carbonyl)-3,4-dihydro-1H-isoquinoline-2-carboxylic acid tert-butyl ester

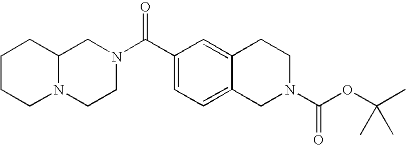

MS (ESI): mass calcd. for C₂₃H₃₃N₃O₃, 399.54; m/z found, 400.3 [M+H]⁺.

### Example 5

### (4-Isopropyl-[1,4]diazepan-1-yl)-(1,2,3,4-tetrahydro-isoquinolin-6-yl)-methanone

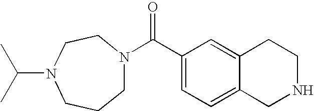

To a solution of 6-(4-isopropyl-[1,4]diazepane-1-carbonyl)-3,4-dihydro-1H-isoquinoline-2-carboxylic acid tert-butyl ester (1.13 g, 2.81 mmol) in DCM (21 mL) was added TFA (9 mL). After 2 h, the solution was concentrated and the resulting residue was dissolved in MeOH (30 mL) and treated with DOWEX® Monosphere 550A (OH) Anion Exchange Resin (DOWEX® resin). After 2 h, the suspension was filtered and concentrated and the residue was purified by FCC to provide 400 mg (47%) of the title compound as an yellow gum. MS (ESI): mass calcd. for C₁₈H₂₇N₃O, 301.22; m/z found, 302.2 [M+H]⁺. ¹H NMR (CDCl₃; mixture of rotamers): 7.15 (d, J=7.9, 1H), 7.14 (s, 1H), 7.04 (d, J=7.9, 1H), 4.06 (s, 2H), 3.81-3.74 (m, 2H), 3.47-3.44 (m, 2H), 3.19 (t, J=5.8, 2H), 3.08 (sept, J=6.5, 0.5H), 2.96-2.88 (m, 1.5H), 2.86 (t, J=5.8, 2H), 2.72-2.69 (m, 2H), 2.61-2.59 (m, 1H), 1.94 (p, J=5.7, 1H), 1.85-1.81 (bm, 1H), 1.09 (d, J=6.5, 3H), 1.00 (d, J=6.5, 3H).

### Example 6

### Piperidin-1-yl-(1,2,3,4-tetrahydro-isoquinolin-6-yl)-methanone

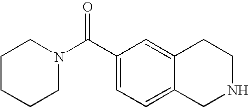

Step A: 6-(Piperidine-1-carbonyl)-3,4-dihydro-1H-isoquinoline-2-carboxylic acid tert-butyl ester. The title compound was prepared using methods analogous to those described in Example 1, Steps B-D. MS (ESI): mass calcd. for C₂₀H₂₈N₂O₃, 344.21; m/z found, 345.2 [M+H]⁺.

Step B. The title compound was prepared as described in Example 7. MS (ESI): mass calcd. for C₁₅H₂₀N₂O, 244.16; m/z found, 245.2 [M+H]⁺.

The compounds in Examples 7-10 were prepared using methods analogous to those described for Example 8.

### Example 7

### Morpholin-4-yl-(1,2,3,4-tetrahydro-isoquinolin-6-yl)-methanone

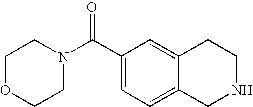

Step A: 6-(Morpholine-4-carbonyl)-3,4-dihydro-1H-isoquinoline-2-carboxylic acid tert-butyl ester. MS (ESI): mass calcd. for C₁₉H₂₆N₂O₄, 346.19; m/z found, 347.2 [M+H]⁺.

Step B. MS (ESI): mass calcd. for C₁₄H₁₈N₂O₂, 246.14; m/z found, 247.2 [M+H]⁺.

### Example 8

### (4-Cyclopentyl-piperazin-1-yl)-(1,2,3,4-tetrahydro-isoquinolin-6-yl)-methanone

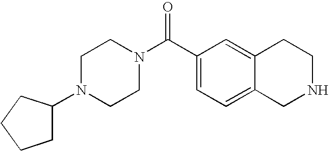

MS (ESI): mass calcd. for C₁₉H₂₇N₃O, 313.45; m/z found, 314.2 [M+H]⁺.

### Example 9

### (4-Cyclohexyl-piperazin-1-yl)-(1,2,3,4-tetrahydro-isoquinolin-6-yl)-methanone

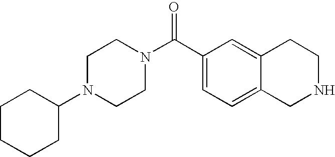

MS (ESI): mass calcd. for C₂₀H₂₉N₃O, 327.47; m/z found, 428.2 [M+H]⁺.

### Example 10

### (Octahydro-pyrido[1,2-a]pyrazin-2-yl)-(1,2,3,4-tetrahydro-isoquinolin-6-yl)-methanone

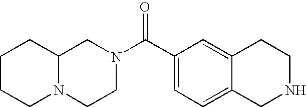

MS (ESI): mass calcd. for C₁₈H₂₅N₃O, 299.42; m/z found, 300.2 [M+H]⁺.

### Example 11

### (2-Benzyl-1,2,3,4-tetrahydro-isoquinolin-6-yl)-(4-isopropyl-[1,4]diazepan-1-yl)-methanone

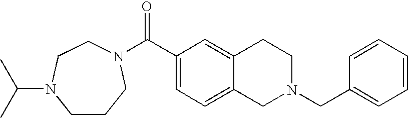

A mixture of acetic acid (46 μL, 0.83 mmol), benzaldehyde (88 μL, 0.83 mmol), and (4-isopropyl-[1,4]diazepan-1-yl)-(1,2,3,4-tetrahydro-isoquinolin-6-yl)-methanone (125 mg, 0.415 mmol) in DCE (4 mL) was stirred at rt for 1 h, and then was treated with NaBH(OAc)₃ (176 mg, 0.830 mmol). After 15 h, the reaction was quenched with saturated (satd.) aqueous (aq.) NaHCO₃ (5 mL) and extracted with DCM (3×5 mL). The combined organic layers were washed with brine, dried and concentrated. The resulting yellow oil was purified by FCC to provide 103 mg (64%) of the title compound as a colorless gum. MS (ESI): mass calcd. for C₂₅H₃₃N₃O, 391.26; m/z found, 392.3 [M+H]⁺. ¹H NMR (CDCl₃; mixture of rotamers): 7.39 (d, J=7.1, 2H). 7.34 (t, J=7.1, 2H), 7.30-7.26 (m, 1H), 7.13 (s, 1H), 7.10 (d, J=7.9, 1H), 6.99 (d, J=7.9, 1H), 3.75-3.73 (m, 2H), 3.69 (s, 2H) 3.63 (s, 2H), 3.44-3.40 (m, 2H), 2.96-2.85 (m, 3H), 2.78 (t, J=5.1, 1H), 2.75 (t, J=5.9, 2H), 2.67 (t, J=5.8, 1H), 2.59 (t, J=5.6, 1H), 2.55 (t, J=5.1, 1H), 1.90 (p, J=5.8, 1H), 1.69 (p, J=5.6, 1H), 1.02 (d, J=6.6, 3H), 0.97 (d, J=6.6, 3H).

The compounds in Examples 12-13 were prepared using methods analogous to those described for Example 11.

### Example 12

### (2-Benzyl-1,2,3,4-tetrahydro-isoquinolin-6-yl)-piperidin-1-yl-methanone

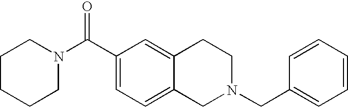

MS (ESI): mass calcd. for C₂₂H₂₆N₂O, 334.20; m/z found, 335.2 [M+H]⁺. ¹H NMR (CDCl₃): 7.39, (d, J=7.1, 2H), 7.33 (t, J=7.1, 2H), 7.27 (t, J=7.1, 1H), 7.14 (s, 1H), 7.10 (d, J=7.8, 1H), 6.99 (d, J=7.8, 1H), 3.69 (s, 2H), 3.68 (bs, 2H), 3.63 (s, 2H), 3.33 (bs, 2H), 2.90 (t, J=5.9, 2H), 2.75 (t, J=5.9, 2H), 1.68-1.60 (m, 4H), 1.49 (bs, 2H).

### Example 13

### (2-Benzyl-1,2,3,4-tetrahydro-isoquinolin-6-yl)-morpholin-4-yl-methanone

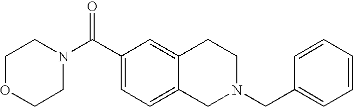

MS (ESI): mass calcd. for C₂₁H₂₄N₂O₂, 336.18; m/z found, 337.2 [M+H]⁺.

### Example 14

### (4-Cyclobutyl-piperazin-1-yl)-[2-(4-trifluoromethyl-benzyl)-1,2,3,4-tetrahydro-isoquinolin-6-yl]-methanone

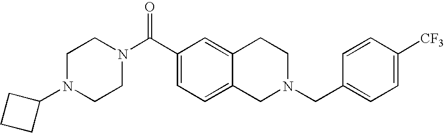

Step A: 2-(4-Trifluoromethyl-benzyl)-1,2,3,4-tetrahydro-isoquinoline-6-carboxylic acid methyl ester. The title compound was prepared using methods analogous to those described in Example 11 to give a pale yellow oil, which was used in the next step without further purification. MS (ESI): mass calcd. for C₁₉H₁₈F₃NO₂, 349.13; m/z found, 350.3 [M+H]⁺.

Step B: Potassium 2-(4-trifluoromethyl-benzyl)-1,2,3,4-tetrahydro-isoquinoline-6-carboxylate. To a solution of 2-(4-trifluoromethyl-benzyl)-1,2,3,4-tetrahydro-isoquinoline-6-carboxylic acid methyl ester (1.27 g crude) in i-PrOH (18 mL) was added 2 N KOH (2.0 mL, 4.0 mmol). The solution was stirred at 80° C. for 16 h and then concentrated to provide 1.27 g (100%) of the title compound as a pale yellow solid. The solid was used in the next step without further purification. MS (ESI): mass calcd. for C₁₈H₁₅F₃KNO₂, 373.07; m/z found, 335.1 [M-K+H]⁺.

Step C: (4-Cyclobutyl-piperazin-1-yl)-[2-(4-trifluoromethyl-benzyl)-1,2,3,4-tetrahydro-isoquinolin-6-yl]-methanone. Potassium 2-(4-trifluoromethyl-benzyl)-1,2,3,4-tetrahydro-isoquinoline-6-carboxylate (318 mg, 0.825 mmol) and EDC (237 mg, 1.24 mmol) were stirred in DMF (8 mL) until the solution was clear. TEA (253 μL, 1.82 mmol) and 1-cyclobutylpiperazine dihydrochloride (194 mg, 0.908 mmol) were added and the solution was stirred at rt for 20 h. After concentrating the reaction mixture, the resulting residue was dissolved in DCM (10 mL) and washed with 1 N NaOH (10 mL). The aqueous layer was extracted with DCM (3×10 mL). The combined organic layers were washed with brine, dried and concentrated. The resulting yellow gum was purified by FCC to provide 198 mg (52%) of the title compound as an orange solid. MS (ESI): mass calcd. for C₂₆H₃₀F₃N₃O, 457.53; m/z found, 458.3 [M+H]⁺. ¹H NMR (CDCl₃): 7.59 (d, J=8.1, 2H), 7.51 (d, J=8.1, 2H), 7.16 (s, 1H), 7.12 (d, J=7.8, 1H), 6.99 (d, J=7.8, 1H), 3.77 (bs, 2H), 3.73 (s, 2H), 3.63 (s, 2H), 3.43 (bs, 2H), 2.91 (t, J=5.7, 2H), 2.76-2.70 (m, 3H), 2.38 (bs, 2H), 2.23 (bs, 2H), 2.06-2.00 (m, 2H), 1.90-1.83 (m, 2H), 1.76-1.65, (m, 2H).

The compounds from Example 15 to Example 26 were prepared using methods analogous to those described for Example 14.

### Example 15

### (4-Cyclobutyl-piperazin-1-yl)-(2-thiophen-3-ylmethyl-1,2,3,4-tetrahydro-isoquinolin-6-yl)-methanone

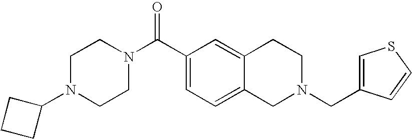

MS (ESI): mass calcd. for C₂₃H₂₉N₃OS, 395.20; m/z found, 396.2 [M+H]⁺. ¹H NMR (CDCl₃): 7.30 (dd, J=4.9, 2.9, 1H), 7.18 (dd, J=7.8, 2.9, 1H), 7.15 (s, 1H), 7.12-7.10 (m, 2H), 7.01 (d, J=7.8, 1H), 3.77 (bs, 2H), 3.72 (s, 2H), 3.64 (s, 2H), 3.43 (bs, 2H), 2.91 (t, J=5.8, 2H), 2.76-2.70 (m, 3H), 2.40 (bs, 2H), 2.23 (bs, 2H), 2.06-2.01 (m, 2H), 1.91-1.83 (m, 2H), 1.76-1.64 (m, 2H).

### Example 16

### (4-Cyclobutyl-piperazin-1-yl)-[2-(3,4-dichloro-benzyl)-1,2,3,4-tetrahydro-isoquinolin-6-yl]-methanone

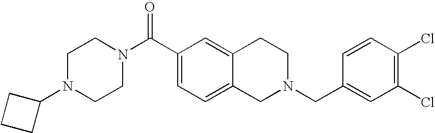

MS (ESI): mass calcd. for C₂₆H₃₀F₃N₃O, 457.23; m/z found, 458.3 [M+H]⁺.

### Example 17

### [2-(4-Chloro-benzyl)-1,2,3,4-tetrahydro-isoquinolin-6-yl]-(4-cyclobutyl-piperazin-1-yl)-methanone

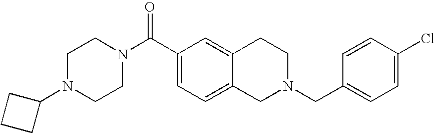

MS (ESI): mass calcd. for C₂₅H₃₀ClN₃O, 423.21; m/z found, 424.2 [M+H]⁺.

### Example 18

### (2-Benzyl-1,2,3,4-tetrahydro-isoquinolin-6-yl)-(4-cyclobutyl-piperazin-1-yl)-methanone

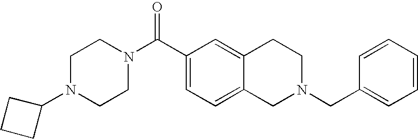

MS (ESI): mass calcd. for C₂₅H₃₁N₃O, 389.25; m/z found, 390.3 [M+H]⁺. ¹H NMR (CDCl₃): 7.38, (d, J=7.2, 2H), 7.34 (t, J=7.2, 2H), 7.28 (t, J=7.2, 1H), 7.15 (s, 1H), 7.11 (d, J=7.8, 1H), 6.99 (d, J=7.8, 1H), 3.77 (bs, 2H), 3.69 (s, 2H), 3.63 (s, 2H), 3.43 (bs, 2H), 2.91 (t, J=5.8, 2H), 2.76-2.70 (m, 3H), 2.78 (bs, 2H), 2.23 (bs, 2H), 2.06-2.01 (m, 2H), 1.91-1.83 (m, 2H), 1.76-1.65 (m, 2H).

### Example 19

### [2-(3,4-Dichloro-benzyl)-1,2,3,4-tetrahydro-isoquinolin-6-yl]-(4-isopropyl-piperazin-1-yl)-methanone

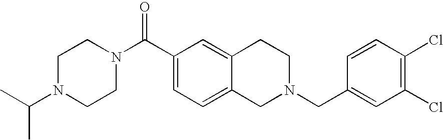

MS (ESI): mass calcd. for C₂₄H₂₉Cl₂N₃O, 445.17; m/z found, 446.2 [M+H]⁺. ¹H NMR (CDCl₃): 7.51 (d, J=1.9, 1H), 7.40 (d, J=8.2, 1H), 7.23 (dd, J=8.2, 0.9, 1H), 7.17 (s, 1H), 7.13 (d, J=7.8, 1H), 7.00 (d, J=7.8, 1H), 3.77 (bs, 2H), 3.63 (s, 2H), 3.62 (s, 2H), 3.44 (bs, 2H), 2.92 (t, J=5.8, 2H), 2.70-2.68 (m, 3H), 2.58 (bs, 2H), 2.44 (bs, 2H), 1.05 (d, J=6.5, 6H).

### Example 20

### (4-Isopropyl-piperazin-1-yl)-[2-(4-trifluoromethyl-benzyl)-1,2,3,4-tetrahydro-isoquinolin-6-yl]-methanone

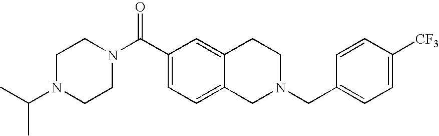

MS (ESI): mass calcd. for C₂₅H₃₀F₃N₃O, 445.23; m/z found, 446.2 [M+H]⁺.

### Example 21

### (2-Benzyl-1,2,34-tetrahydro-isoquinolin-6-yl)-(4-isopropyl-piperazin-1-yl)-methanone

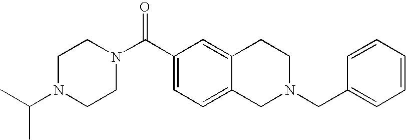

MS (ESI): mass calcd. for C₂₄H₃₁N₃O, 377.25; m/z found, 378.3 [M+H]⁺. ¹H NMR (CDCl₃): 7.39, (d, J=7.2, 2H), 7.34 (t, J=7.2, 2H), 7.29 (t, J=7.2, 1H), 7.16 (s, 1H), 7.12 (d, J=7.8, 1H), 7.00 (d, J=7.8, 1H), 3.77 (bs, 2H), 3.69 (s, 2H), 3.64 (s, 2H), 3.43 (bs, 2H), 2.91 (t, J=5.9, 2H), 2.75 (t, J=5.9, 2H), 2.71 (sept, J=6.5, 1H), 2.57 (bs, 2H), 2.42 (bs, 2H), 1.04 (d, J=6.5, 6H).

### Example 22

### [2-(4-Chloro-benzyl)-1,2,3,4-tetrahydro-isoquinolin-6-yl]-(4-isoproplyl-piperazin-1-yl)-methanone

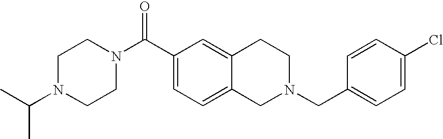

MS (ESI): mass calcd. for C₂₄H₃₀ClN₃O, 411.21; m/z found, 412.2 [M+H]⁺.

### Example 23

### (4-Cyclopropyl-piperazin-1-yl)-[2-(4-trifluoromethyl-benzyl)-1,2,3,4-tetrahydro-isoquinolin-6-yl]-methanone

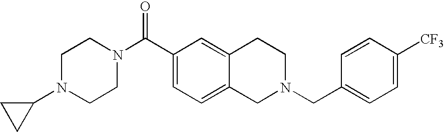

MS (ESI): mass calcd. for C₂₅H₂₈F₃N₃O, 443.22; m/z found, 444.2 [M+H]⁺.

### Example 24

### [2-(4-Chloro-benzyl)-1,2,3,4-tetrahydro-isoquinolin-6-yl]-(4-cyclopropyl-piperazin-1-yl)-methanone

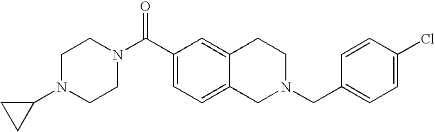

MS (ESI): mass calcd. for C₂₄H₂₈ClN₃O, 409.19; m/z found, 410.2 [M+H]⁺. ¹H NMR (CDCl₃): 7.33-7.27 (m, 4H), 7.17 (s, 1H), 7.12 (d, J=7.8, 1H), 7.00 (d, J=7.8, 1H), 3.73 (bs, 2H), 3.64 (s, 2H), 3.61 (s, 2H), 3.38 (bs, 2H), 2.90 (t, J=5.8, 2H), 2.73 (t, J=5.8, 2H), 2.67 (bs, 2H), 2.53 (bs, 2H), 1.65-1.61 (m, 1H), 0.49-0.45 (m, 2H), 0.44-0.40 (m, 2H).

### Example 25

### 4-[6-(4-Cyclobutyl-piperazine-1-carbonyl)-3,4-dihydro-1H-isoquinolin-2-ylmethyl]-benzonitrile

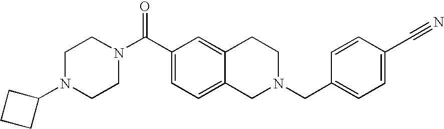

MS (ESI): mass calcd. for C₂₆H₃₀N₄O, 414.24; m/z found, 415.2 [M+H]⁺.

### Example 26

### (2-Benzyl-1,2,3,4-tetrahydro-isoquinolin-7-yl)-(4-cyclobutyl-piperazin-1-yl)-methanone

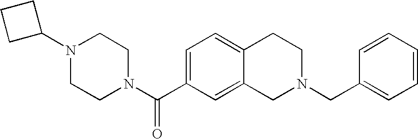

MS (ESI): mass calcd. for C₂₅H₃₁N₃O, 389.25; m/z found, 390.3 [M+H]⁺. ¹H NMR (CDCl₃): 7.39-7.32 (m, 4H), 7.29-7.27 (m, 1H), 7.15-7.10 (m, 2H), 7.03 (s, 1H), 3.76 (bs, 2H), 3.69 (s, 2H), 3.63 (s, 2H), 3.42 (bs, 2H), 2.90 (t, J=5.8, 2H), 2.76-2.70 (m, 3H), 2.37 (bs, 2H), 2.22 (bs, 2H), 2.06-2.00 (m, 2H), 1.90-1.82 (m, 2H), 1.76-1.66 (m, 2H).

### Example 27

### (2-Benzoyl-1,2,3,4-tetrahydro-isoquinolin-6-yl)-(4-cyclobutyl-piperazin-1-yl)-methanone

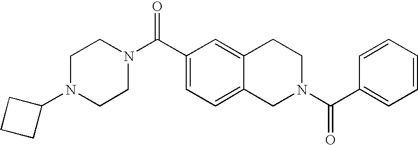

A 0° C. solution of (4-cyclobutyl-piperazin-1-yl)-(1,2,3,4-tetrahydro-isoquinolin-6-yl)-methanone (0.20 g, 0.67 mmol) and TEA (190 μL, 1.4 mmol) in DCM (7 mL) was treated with benzoyl chloride (160 μL, 1.4 mmol), and the reaction was allowed to warm to rt over 18 h. The reaction was quenched with satd. aq. NaHCO₃ (10 mL) and extracted with DCM (2×20 mL). The combined organic layers were washed with brine, dried and concentrated. The resulting residue was purified by reverse phase HPLC to provide 160 mg (60%) of the title compound as a white solid. MS (ESI): mass calcd. for C₂₅H₂₉N₃O₂, 403.23; m/z found, 404.2 [M+H]⁺. ¹H NMR (CDCl₃; mixture of rotamers): 7.45 (s, 5H), 7.26-7.14 (m, 2.6H), 6.92 (bs, 0.4), 4.91 (bs, 1.2H), 4.60 (bs, 0.8H), 4.00-3.45 (m, 6H), 2.98-2.89 (m, 2H), 2.74 (p, J=7.7, 1H), 2.40 (bs, 2H), 2.27 (bs, 2H), 2.07-2.01 (m, 2H), 1.93-1.84 (m, 2H), 1.77-1.66 (m, 2H).

The compounds from Example 28 to Example 104 were prepared using methods analogous to those described for Example 27.

### Example 28

### (2-Benzoyl-1,2,3,4-tetrahydro-isoquinolin-6-yl)-(4-isopropyl-piperazin-1-yl)-methanone

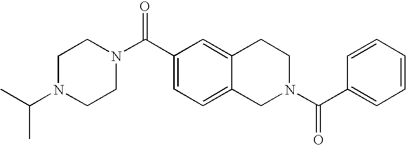

MS (ESI): mass calcd. for C₂₂H₃₃N₃O₃, 387.25; m/z found, 388.3 [M+H]⁺.

### Example 29

### 1-[6-(4-Isopropyl-piperazine-1-carbonyl)-3,4-dihydro-1H-isoquinolin-2-yl]-ethanone

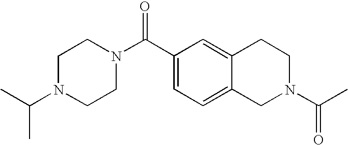

MS (ESI): mass calcd. for C₁₉H₂₇N₃O₂, 329.21; m/z found, 330.2 [M+H]⁺.

### Example 30

### 1-[6-(4-Cyclobutyl-piperazine-1-carbonyl)-3,4-dihydro-1H-isoquinolin-2-yl]-ethanone

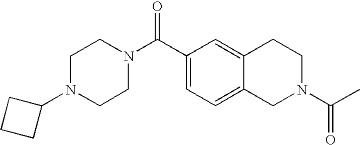

MS (ESI): mass calcd. for C₂₀H₂₇N₃O₂, 341.21; m/z found, 342.2 [M+H]⁺. ¹H NMR (CDCl₃; mixture of rotamers): 7.25-7.13 (m, 3H), 4.75 (s, 1.25H), 4.63 (s, 0.75H), 3.83 (t, J=5.9, 0.75H), 3.79 (bs, 2H), 3.68 (t, J=5.9, 1.25H), 3.45 (bs, 2H), 2.93 (t, J=5.9, 1.25H), 2.86 (t, J=5.9, 0.75H), 2.75 (p, J=7.7, 1H), 2.40 (bs, 2H), 2.26 (bs, 2H), 2.19 (s, 3H), 2.06-2.02 (m, 2H), 1.91-1.85 (m, 2H), 1.76-1.67 (m, 2H).

### Example 31

### Cyclobutyl-[6-(4-cyclobutyl-piperazine-1-carbonyl)-3,4-dihydro-1H-isoquinolin-2-yl]-methanone

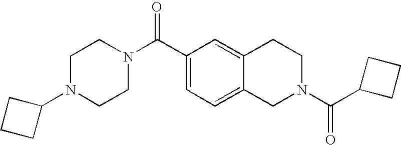

MS (ESI): mass calcd. for C₂₃H₃₁N₃O₂, 381.24; m/z found, 382.3 [M+H]⁺.

### Example 32

### [6-(4-Cyclobutyl-piperazine-1-carbonyl)-3,4-dihydro-1H-isoquinolin-2-yl]-cyclopentyl-methanone

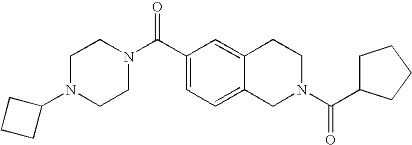

MS (ESI): mass calcd. for C₂₄H₃₃N₃O₂, 395.26; m/z found, 396.3 [M+H]⁺.

### Example 33

### [6-(4-Cyclobutyl-piperazine-1-carbonyl)-3,4-dihydro-1H-isoquinolin-2-yl]-cyclohexyl-methanone

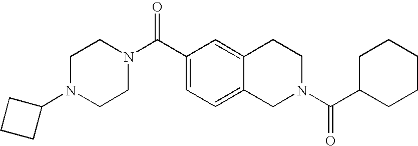

MS (ESI): mass calcd. for C₂₅H₃₅N₃O₂, 409.27; m/z found, 410.3 [M+H]⁺. ¹H NMR (CDCl₃; mixture of rotamers): 7.23-7.14 (m, 3H), 4.73 (s, 1.2H), 4.67 (s, 0.8H), 3.82 (t, J=5.8, 0.8H), 3.78 (bs, 2H), 3.72 (t, J=5.8, 1.2H), 3.44 (bs, 2H), 2.92 (t, J=5.8, 1.2H), 2.85 (t, J=5.8, 0.8H), 2.75 (p, J=7.8, 1H), 2.55 (tt, J=11.6, 3.3, 1H), 2.40 (bs, 2H), 2.26 (bs, 2H), 2.06-2.01 (m, 2.2H), 1.91-1.67 (m, 9.8H), 1.60-1.51 (m, 4H).

### Example 34

### [6-(4-Cyclobutyl-piperazine-1-carbonyl)-3,4-dihydro-1H-isoquinolin-2-yl]-cyclopropyl-methanone

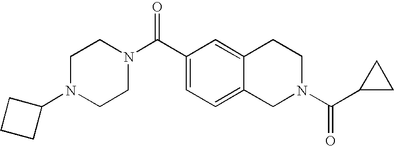

MS (ESI): mass calcd. for C₂₂H₂₉N₃O₂, 367.23; m/z found, 368.2 [M+H]⁺.

### Example 35

### [6-(4-Cyclobutyl-[1,4]diazepane-1-carbonyl)-3,4-dihydro-1H-isoquinolin-2-yl]-phenyl-methanone

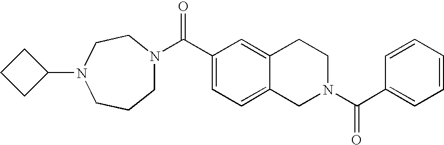

MS (ESI): mass calcd. for C₂₆H₃₁N₃O₂, 417.24; m/z found, 418.3 [M+H]⁺. ¹H NMR (CDCl₃; mixture of rotamers): 7.44 (s, 5H), 7.22-7.16 (m, 2.6H), 6.92 (bs, 0.4H), 4.90 (bs, 1.2H), 4.59 (bs, 0.8H), 3.99 (bs, 0.8H), 3.77-3.75 (m, 2H), 3.64 (bs, 1.2H), 3.49-3.44 (m, 2H), 2.97-2.82 (m, 3H), 2.62-2.61 (m, 1H), 2.51-2.49 (m, 1H), 2.44-2.40 (m, 2H), 2.08-1.93 (m, 3H), 1.87-1.74 (m, 3H), 1.72-1.57 (m, 2H).

### Example 36

### [7-(4-Cyclobutyl-[1,4]diazepane-1-carbonyl)-3,4-dihydro-1H-isoquinolin-2-yl]-phenyl-methanone

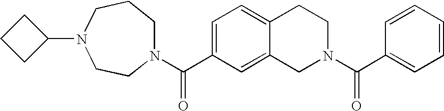

MS (ESI): mass calcd. for C₂₆H₃₁N₃O₂, 417.24; m/z found, 418.3 [M+H]⁺. ¹H NMR (CDCl₃; mixture of rotamers): 7.44 (s, 5H), 7.22-7.16 (m, 2.6H), 6.96 (bs, 0.4H), 4.90 (bs, 1.2H), 4.59 (bs, 0.8H), 3.99 (bs, 0.8H), 3.76-3.64 (m, 3.2H), 3.51-3.41 (m, 2H), 2.98-2.87 (m, 3H), 2.62 (bs, 1H), 2.50-2.42 (m, 3H), 2.04-1.95 (m, 3H), 1.86-1.75 (m, 3H), 1.70-1.61 (m, 2H).

### Example 37

### [7-(4-Cyclobutyl-[1,4]diazepane-1-carbonyl)-3,4-dihydro-1H-isoquinolin-2-yl]-cyclopentyl-methanone

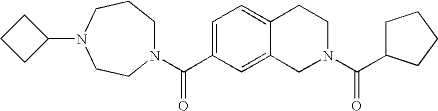

MS (ESI): mass calcd. for C₂₅H₃₅N₃O₂, 409.27; m/z found, 410.3 [M+H]⁺.

### Example 38

### [7-(4-Cyclobutyl-[1,4]diazepane-1-carbonyl)-3,4-dihydro-1H-isoquinolin-2-yl]-cyclohexyl-methanone

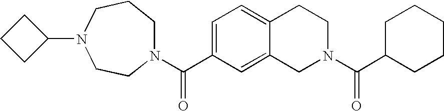

MS (ESI): mass calcd. for C₂₆H₃₇N₃O₂, 423.29; m/z found, 424.3 [M+H]⁺.

### Example 39

### [6-(4-Cyclobutyl-[1,4]diazepane-1-carbonyl)-3,4-dihydro-1H-isoquinolin-2-yl]-cyclopentyl-methanone

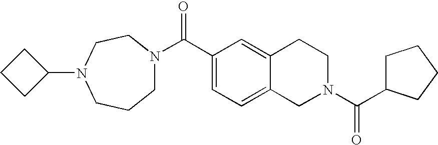

MS (ESI): mass calcd. for C₂₅H₃₅N₃O₂, 409.27; m/z found, 410.3 [M+H]⁺.

### Example 40

### [6-(4-Cyclobutyl-[1,4]diazepane-1-carbonyl)-3,4-dihydro-1H-isoquinolin-2-yl]-cyclohexyl-methanone

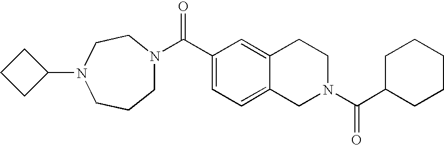

MS (ESI): mass calcd. for C₂₆H₃₇N₃O₂, 423.29; m/z found, 423.3 [M+H]⁺.

### Example 41

### 1-[6-(4-Cyclobutyl-piperazine-1-carbonyl)-3,4-dihydro-1H-isoquinolin-2-yl]-2,2-dimethyl-propan-1-one

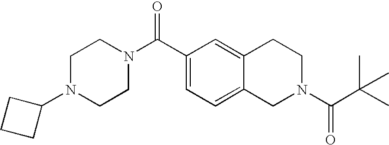

MS (ESI): mass calcd. for C₂₃H₃₃N₃O₂, 383.26; m/z found, 384.3 [M+H]⁺.

### Example 42

### (2-Chloro-phenyl)-[6-(4-cyclobutyl-piperazine-1-carbonyl)-3,4-dihydro-1H-isoquinolin-2-yl]-methanone

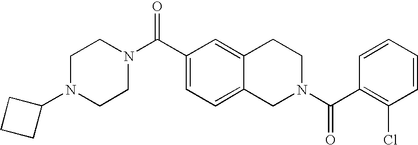

MS (ESI): mass calcd. for C₂₅H₂₈N₃O₂, 437.19; m/z found, 438.2 [M+H]⁺.

### Example 43

### 1-[6-(4-Cyclobutyl-piperazine-1-carbonyl)-3,4-dihydro-1H-isoquinolin-2-yl]-2-cyclopentyl-ethanone

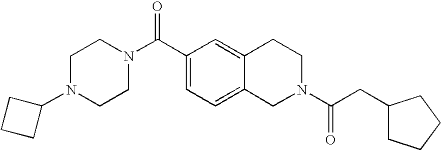

MS (ESI): mass calcd. for C₂₅H₃₅N₃O₂, 409.27; m/z found, 410.3 [M+H]⁺. ¹H NMR (CDCl₃; mixture of rotamers): 7.23-7.12 (m, 3H), 4.75 (s, 1.2H), 4.64 (s, 0.8H), 3.83 (t, J=5.8, 0.8H), 3.78 (bs, 2H), 3.70 (t, J=5.8, 1.2H), 3.44 (bs, 2H), 2.91 (t, J=5.8, 1.2H), 2.85 (t, J=5.8, 0.8H), 2.75 (p, J=7.8, 1H), 2.43 (d, J=7.2, 2H), 2.39 (bs, 2H), 2.33-2.25 (m, 3H), 2.07-2.01 (m, 2H), 1.91-1.83 (m, 4H), 1.75-1.56 (m, 6H), 1.22-1.14 (m, 2H).

### Example 44

### [6-(4-Cyclobutyl-piperazine-1-carbonyl)-3,4-dihydro-1H-isoquinolin-2-yl]-furan-3-yl-methanone

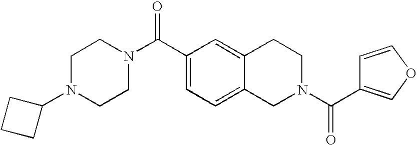

MS (ESI): mass calcd. for C₂₃H₂₇N₃O₃, 393.21; m/z found, 394.2 [M+H]⁺.

### Example 45

### (S)-1-[6-(2-Pyrrolidin-1-ylmethyl-pyrrolidine-1-carbonyl)-3,4-dihydro-1H-isoquinolin-2-yl]-propan-1-one

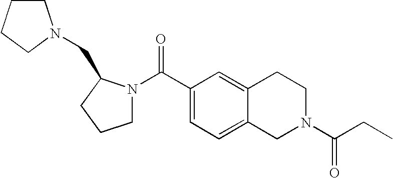

MS (ESI): mass calcd. for C₂₂H₃₁N₃O₂, 369.24; m/z found, 370.3 [M+H]⁺. ¹H NMR (CDCl₃; mixture of rotamers): 7.33-7.29 (m, 2H), 7.17-7.11 (m, 1H), 4.79-4.71 (m, 1.2H), 4.63 (s, 0.8H), 4.42 (bs, 0.8H), 4.01-3.41 (m, 4.2H), 2.91-2.85 (m, 2.8H), 2.63 (bs, 3.2H), 2.44 (q, J=7.5, 2H), 2.26-1.60 (m, 10H), 1.21-1.17 (m, 3H).

### Example 46

### (S)-1-[6-(2-Pyrrolidin-1-ylmethyl-pyrrolidine-1-carbonyl)-3,4-dihydro-1H-isoquinolin-2-yl]-butan-1-one

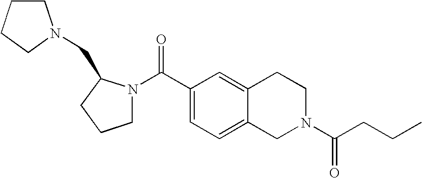

MS (ESI): mass calcd. for C₂₃H₃₃N₃O₂, 383.26; m/z found, 384.3 [M+H]⁺.

### Example 47

### (S)-2,2-Dimethyl-1-[6-(2-pyrrolidin-1-ylmethyl-pyrrolidine-1-carbonyl)-3,4-dihydro-1H-isoquinolin-2-yl]-propan-1-one

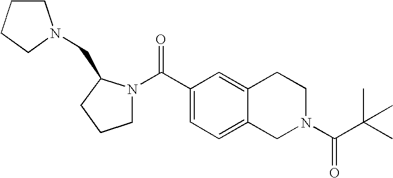

MS (ESI): mass calcd. for C₂₄H₃₅N₃O₂, 397.27; m/z found, 398.3 [M+H]⁺.

### Example 48

### (S)-Phenyl-[6-(2-pyrrolidin-1-ylmethyl-pyrrolidine-1-carbonyl)-3,4-dihydro-1H-isoquinolin-2-yl]-methanone

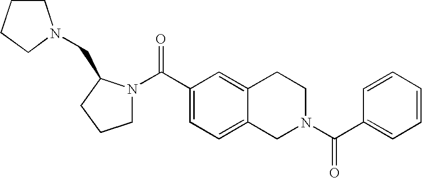

MS (ESI): mass calcd. for C₂₆H₃₁N₃O₂, 417.24; m/z found, 418.3 [M+H]⁺. ¹H NMR (CDCl₃; mixture of rotamers): 7.45 (s, 5H), 7.36-7.21 (m, 2.6H), 6.93 (bs, 0.4H), 4.91 (bs, 1.2H), 4.60 (bs, 0.8H), 4.42 (bs, 0.8H), 4.01 (bs, 1H), 3.72-3.42 (m, 3.2H), 2.99-2.88 (m, 2.8H), 2.63 (bs, 3.2H), 2.41-1.61 (m, 10H).

### Example 49

### (S)-(4-tert-Butyl-phenyl)-[6-(2-pyrrolidin-1-ylmethyl-pyrrolidine-1-carbonyl)-3,4-dihydro-1H-isoquinolin-2-yl]-methanone

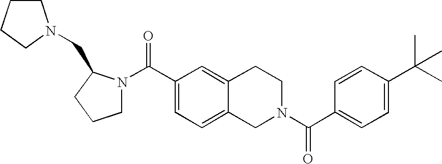

MS (ESI): mass calcd. for C₃₀H₃₉N₃O₂, 473.30; m/z found, 474.3 [M+H]⁺.

### Example 50

### (S)-(2-Chloro-phenyl)-[6-(2-pyrrolidin-1-ylmethyl-pyrrolidine-1-carbonyl)-3,4-dihydro-1H-isoquinolin-2-yl]-methanone

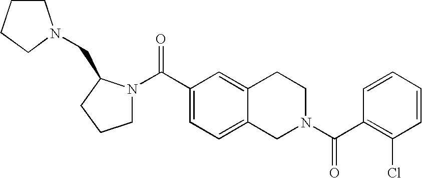

MS (ESI): mass calcd. for C₂₆H₃₀ClN₃O₂, 451.20; m/z found, 452.2 [M+H]⁺.

### Example 51

### (S)-(3-Chloro-phenyl)-[6-(2-pyrrolidin-1-ylmethyl-pyrrolidine-1-carbonyl)-3,4-dihydro-1H-isoquinolin-2-yl]-methanone

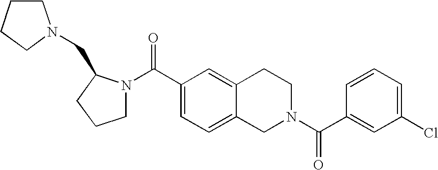

MS (ESI): mass calcd. for C₂₆H₃₀ClN₃O₂, 451.20; m/z found, 452.2 [M+H]⁺.

### Example 52

### (S)-3-[6-(2-Pyrrolidin-1-ylmethyl-pyrrolidine-1-carbonyl)-3,4-dihydro-1H-isoquinoline-2-carbonyl]-benzonitrile

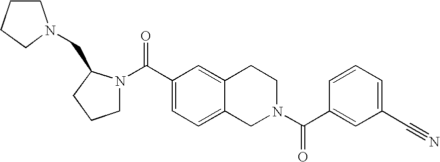

MS (ESI): mass calcd. for C₂₇H₃₀N₄O₂, 442.24; m/z found, 443.3 [M+H]⁺.

### Example 53

### (S)-4-[6-(2-Pyrrolidin-1-ylmethyl-pyrrolidine-1-carbonyl)-3,4-dihydro-1H-isoquinoline-2-carbonyl]-benzonitrile

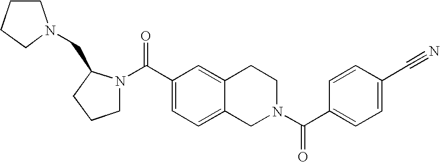

MS (ESI): mass calcd. for C₂₇H₃₀N₄O₂, 442.24; m/z found, 443.3 [M+H]⁺.

### Example 54

### (S)-[6-(2-Pyrrolidin-1-ylmethyl-pyrrolidine-1-carbonyl)-3,4-dihydro-1H-isoquinolin-2-yl]-o-tolyl-methanone

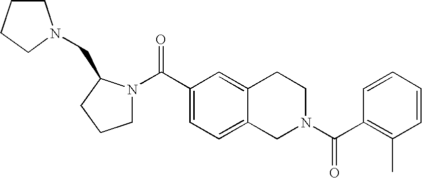

MS (ESI): mass calcd. for C₂₇H₃₃N₃O₂, 431.26; m/z found, 432.3 [M+H]⁺.

### Example 55

### (S)-[6-(2-Pyrrolidin-1-ylmethyl-pyrrolidine-1-carbonyl)-3,4-dihydro-1H-isoquinolin-2-yl]-p-tolyl-methanone

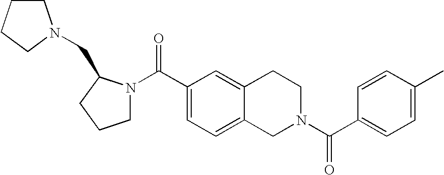

MS (ESI): mass calcd. for C₂₇H₃₃N₃O₂, 431.26; m/z found, 432.3 [M+H]⁺.

### Example 56

### (S)-(2-Fluoro-phenyl)-[6-(2-Pyrrolidin-1-ylmethyl-pyrrolidine-1-carbonyl)-3,4-dihydro-1H-isoquinolin-2-yl]-methanone

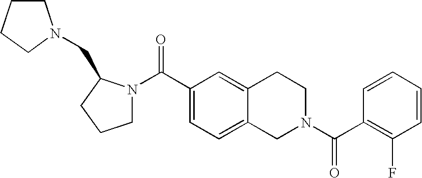

MS (ESI): mass calcd. for C₂₆H₃₀FN₃O₂, 435.23; m/z found, 436.3 [M+H]⁺.

### Example 57

### (S)-(3-Fluoro-phenyl)-[6-(2-pyrrolidin-1-ylmethyl-pyrrolidine-1-carbonyl)-3,4-dihydro-1H-isoquinolin-2-yl]-methanone

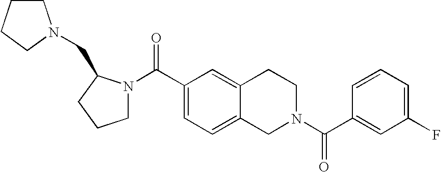

MS (ESI): mass calcd. for C₂₆H₃₀FN₃O₂, 435.23; m/z found, 436.3 [M+H]⁺.

### Example 58

### (S)-(4-Fluoro-phenyl)-[6-(2-pyrrolidin-1-ylmethyl-pyrrolidine-1-carbonyl)-3,4-dihydro-1H-isoquinolin-2-yl]-methanone

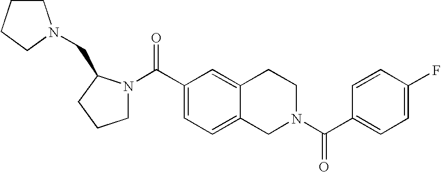

MS (ESI): mass calcd. for C₂₆H₃₀FN₃O₂, 435.23; m/z found, 436.2 [M+H]⁺.

### Example 59

### (S)-(3-Methoxy-phenyl)-[6-(2-Pyrrolidin-1-ylmethyl-pyrrolidine-1-carbonyl)-3,4-dihydro-1H-isoquinolin-2-yl]-methanone

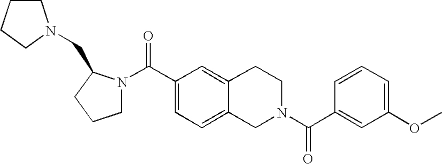

MS (ESI): mass calcd. for C₂₇H₃₃N₃O₃, 447.25; m/z found, 448.3 [M+H]⁺.

### Example 60

### (S)-(4-Methoxy-phenyl)-[6-(2-pyrrolidin-1-ylmethyl-pyrrolidine-1-carbonyl)-3,4-dihydro-1H-isoquinolin-2-yl]-methanone

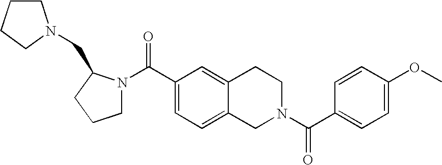

MS (ESI): mass calcd. for C₂₇H₃₃N₃O₃, 447.25; m/z found, 448.3 [M+H]⁺.

### Example 61

### (S)-2-Phenyl-1-[6-(2-Pyrrolidin-1-ylmethyl-pyrrolidine-1-carbonyl)-3,4-dihydro-1H-isoquinolin-2-yl]-ethanone

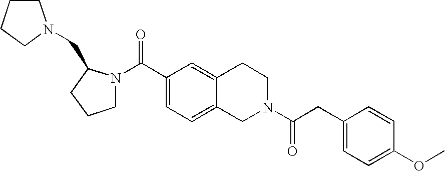

MS (ESI): mass calcd. for C₂₈H₃₅N₃O₃, 461.27; m/z found, 462.3 [M+H]⁺.

### Example 62

### (4-Cyclobutyl-piperazin-1-yl)-[2-(3-fluoro-benzoyl)-1,2,3,4-tetrahydro-isoquinolin-6-yl]-methanone

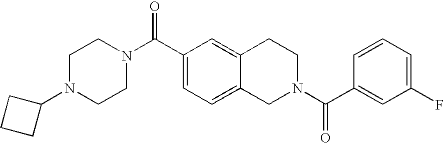

MS (ESI): mass calcd. for C₂₅H₂₈FN₃O₂, 421.22; m/z found, 422.2 [M+H]⁺.

### Example 63

### 1-[6-(4-Cyclopentyl-piperazine-1-carbonyl)-3,4-dihydro-1H-isoquinolin-2-yl]-propan-1-one

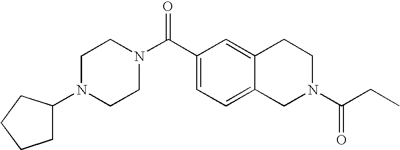

MS (ESI): mass calcd. for C₂₂H₃₁N₃O₂, 369.51; m/z found, 370.3 [M+H]⁺.

### Example 64

### 1-[6-(4-Cyclopentyl-piperazine-1-carbonyl)-3,4-dihydro-1H-isoquinolin-2-yl]-2,2-dimethyl-propan-1-one

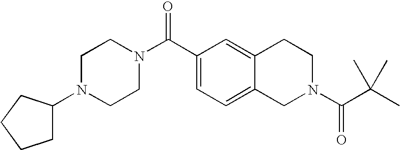

MS (ESI): mass calcd. for C₂₄H₃₅N₃O₂, 397.57; m/z found, 398.3 [M+H]⁺.

### Example 65

### (2-Benzoyl-1,2,3,4-tetrahydro-isoquinolin-6-yl)-(4-cyclopentyl-piperazin-1-yl)-methanone

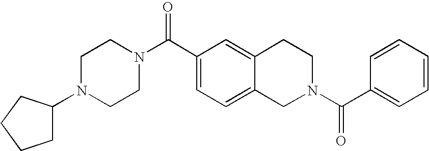

MS (ESI): mass calcd. for C₂₆H₃₁N₃O₂, 417.56; m/z found, 418.3 [M+H]⁺.

### Example 66

### (4-Cyclopentyl-piperazin-1-yl)-[2-(2-fluoro-benzoyl)-1,2,3,4-tetrahydro-isoquinolin-6-yl]-methanone

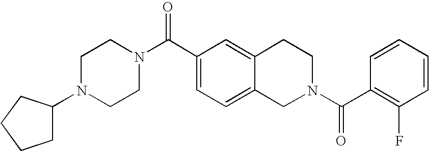

MS (ESI): mass calcd. for C₂₆H₃₀FN₃O₂, 435.55; m/z found, 436.2 [M+H]⁺.

### Example 67

### (4-Cyclopentyl-piperazin-1-yl)-[2-(4-fluoro-benzoyl)-1,2,3,4-tetrahydro-isoquinolin-6-yl]-methanone

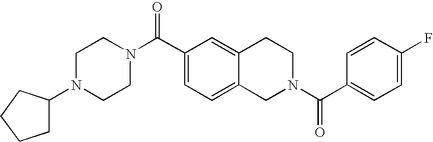

MS (ESI): mass calcd. for C₂₆H₃₀FN₃O₂, 435.55; m/z found, 436.2 [M+H]⁺.

### Example 68

### [2-(2-Chloro-benzoyl)-1,2,3,4-tetrahydro-isoquinolin-6-yl]-(4-cyclopentyl-piperazin-1-yl)-methanone

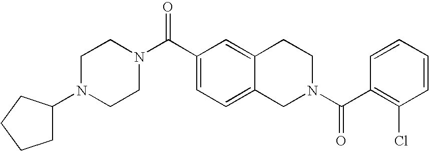

MS (ESI): mass calcd. for C₂₆H₃₀ClN₃O₂, 452.00; m/z found, 453.2 [M+H]⁺.

### Example 69

### [2-(4-Chloro-benzoyl)-1,2,3,4-tetrahydro-isoquinolin-6-yl]-(4-cyclopentyl-piperazin-1-yl)-methanone

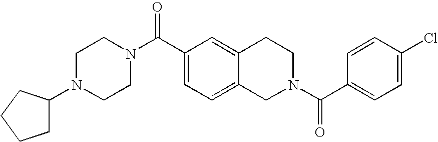

MS (ESI): mass calcd. for C₂₆H₃₀ClN₃O₂, 452.00; m/z found, 453.2 [M+H]⁺.

### Example 70

### (4-Cyclopentyl-piperazin-1-yl)-[2-(4-methyl-benzoyl)-1,2,3,4-tetrahydro-isoquinolin-6-yl]-methanone

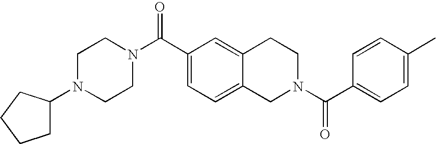

MS (ESI): mass calcd. for C₂₇H₃₃N₃O₂, 431.58; m/z found, 432.3 [M+H]⁺.

### Example 71

### 1-[6-(4-Cyclopentyl-piperazine-1-carbonyl)-3,4-dihydro-1H-isoquinolin-2-yl]-2-(4-fluoro-phenyl)-ethanone

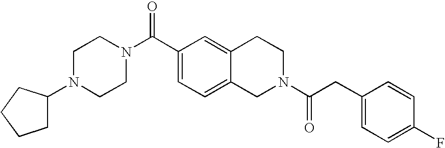

MS (ESI): mass calcd. for C₂₇H₃₂FN₃O₂, 449.57; m/z found, 450.3 [M+H]⁺.

### Example 72

### (4-Cyclohexyl-piperazin-1-yl)-[2-(2-fluoro-benzoyl)-1,2,3,4-tetrahydro-isoquinolin-6-yl]-methanone

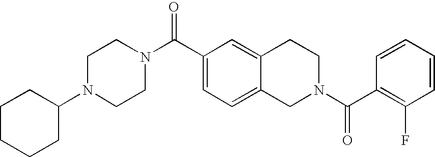

MS (ESI): mass calcd. for C₂₇H₃₂FN₃O₂, 449.57; m/z found, 450.3 [M+H]⁺.

### Example 73

### (4-Cyclohexyl-piperazin-1-yl)-[2-(3-fluoro-benzoyl)-1,2,3,4-tetrahydro-isoquinolin-6-yl]-methanone

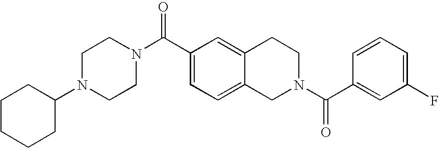

MS (ESI): mass calcd. for C₂₇H₃₂FN₃O₂, 449.57; m/z found, 450.3 [M+H]⁺.

### Example 74

### (4-Cyclohexyl-piperazin-1-yl)-[2-(4-fluoro-benzoyl)-1,2,3,4-tetrahydro-isoquinolin-6-yl]-methanone

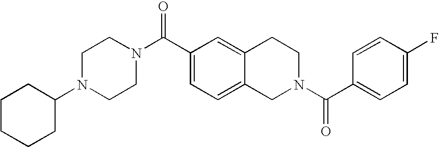

MS (ESI): mass calcd. for C₂₇H₃₂FN₃O₂, 449.57; m/z found, 450.3 [M+H]⁺.

### Example 75

### [2-(2-Chloro-benzoyl)-1,2,3,4-tetrahydro-isoquinolin-6-yl]-(4-cyclohexyl-piperazin-1-yl)-methanone

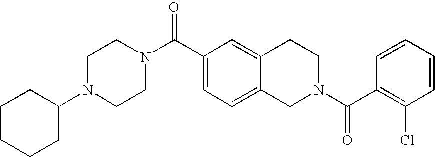

MS (ESI): mass calcd. for C₂₇H₃₂ClN₃O₂, 466.03; m/z found, 466.3 [M]⁺.

### Example 76

### [2-(3-Chloro-benzoyl)-1,2,3,4-tetrahydro-isoquinolin-6-yl]-(4-cyclohexyl-piperazin-1-yl)-methanone

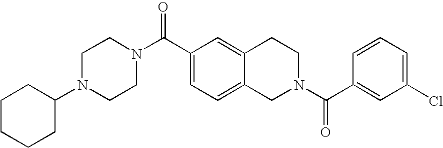

MS (ESI): mass calcd. for C₂₇H₃₂ClN₃O₂, 466.03; m/z found, 466.2 [M]⁺.

### Example 77

### [2-(4-Chloro-benzoyl)-1,2,3,4-tetrahydro-isoquinolin-6-yl]-(4-cyclohexyl-piperazin-1-yl)-methanone

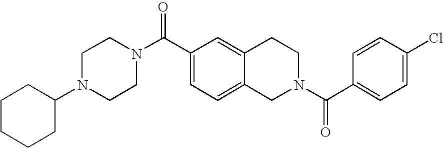

MS (ESI): mass calcd. for C₂₇H₃₂ClN₃O₂, 466.03; m/z found, 466.2 [M]⁺.

### Example 78

### (4-Cyclohexyl-piperazin-1-yl)-[2-(2-methoxy-benzoyl)-1,2,3,4-tetrahydro-isoquinolin-6-yl]-methanone

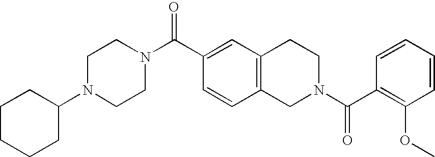

MS (ESI): mass calcd. for C₂₈H₃₅N₃O₃, 461.61; m/z found, 462.3 [M+H]⁺.

### Example 79

### (4-Cyclohexyl-piperazin-1-yl)-[2-(3-methoxy-benzoyl)-1,2,3,4-tetrahydro-isoquinolin-6-yl]-methanone

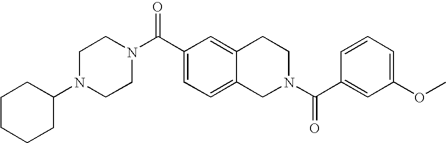

MS (ESI): mass calcd. for C₂₈H₃₅N₃O₃, 461.61; m/z found, 462.3 [M+H]⁺.

### Example 80

### (3-[6-(4-Cyclohexyl-piperazine-1-carbonyl)-3,4-dihydro-1H-isoquinoline-2-carbonyl]-benzonitrile

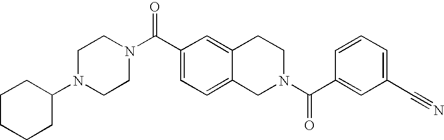

MS (ESI): mass calcd. for C₂₈H₃₂N₄O₂, 456.59; m/z found, 457.3 [M+H]⁺.

### Example 81

### 4-[6-(4-Cyclohexyl-piperazine-1-carbonyl)-3,4-dihydro-1H-isoquinoline-2-carbonyl]-benzonitrile

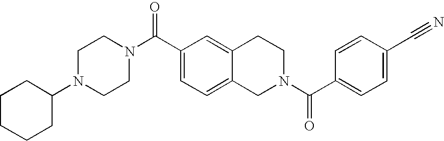

MS (ESI): mass calcd. for C₂₈H₃₂N₄O₂, 456.59; m/z found, 457.3 [M+H]⁺.

### Example 82

### (4-Cyclohexyl-piperazin-1-yl)-[2-(2-methyl-benzoyl)-1,2,3,4-tetrahydro-isoquinolin-6-yl]-methanone

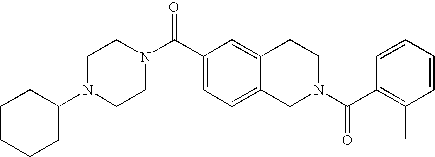

MS (ESI): mass calcd. for C₂₈H₃₅N₃O₂, 445.61; m/z found, 446.3 [M+H]⁺.

### Example 83

### (4-Cyclohexyl-piperazin-1-yl)-[2-(4-methyl-benzoyl)-1,2,3,4-tetrahydro-isoquinolin-6-yl]-methanone

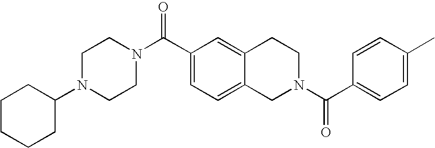

MS (ESI): mass calcd. for C₂₈H₃₅N₃O₂, 445.61; m/z found, 446.3 [M+H]⁺.

### Example 84

### [2-(4-tert-Butyl-benzoyl)-1,2,3,4-tetrahydro-isoquinolin-6-yl]-(4-cyclohexyl-piperazin-1-yl)-methanone

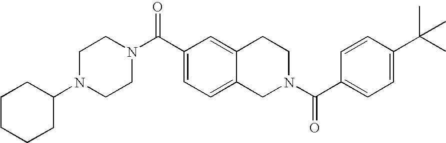

MS (ESI): mass calcd. for C₃₁H₄₁N₃O₂, 487.69; m/z found, 488.3 [M+H]⁺.

### Example 85

### (2-Benzoyl-1,2,3,4-tetrahydro-isoquinolin-6-yl)-(4-cyclohexyl-piperazin-1-yl)-methanone

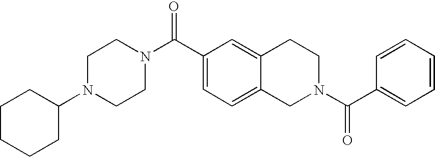

MS (ESI): mass calcd. for C₂₇H₃₃N₃O₂, 431.58; m/z found, 432.3 [M+H]⁺.

### Example 86

### [2-(2-Fluoro-benzoyl)-1,2,3,4-tetrahydro-isoquinolin-6-yl]-(octahydro-pyrido[1,2-a]pyrazin-2-yl)-methanone

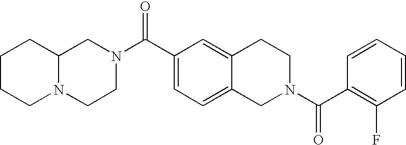

MS (ESI): mass calcd. for C₂₅H₂₈FN₃O₂, 421.52; m/z found, 422.2 [M+H]⁺.

### Example 87

### [2-(3-Fluoro-benzoyl)-1,2,3,4-tetrahydro-isoquinolin-6-yl]-(octahydro-pyrido[1,2-a]pyrazin-2-yl)-methanone

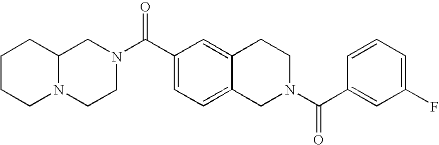

MS (ESI): mass calcd. for C₂₅H₂₈FN₃O₂, 421.52; m/z found, 422.2 [M+H]⁺.

### Example 88

### [2-(4-Fluoro-benzoyl)-1,2,3,4-tetrahydro-isoquinolin-6-yl]-(octahydro-pyrido[1,2-a]pyrazin-2-yl)-methanone

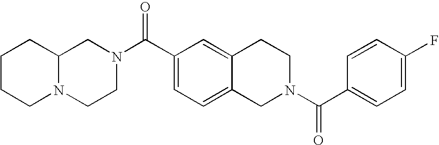

MS (ESI): mass calcd. for C₂₅H₂₈FN₃O₂, 421.52; m/z found, 422.2 [M+H]⁺.

### Example 89

### [2-(2-Chloro-benzoyl)-1,2,3,4-tetrahydro-isoquinolin-6-yl]-(octahydro-pyrido[1,2-a]pyrazin-2-yl)-methanone

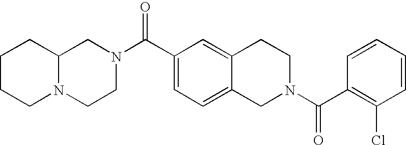

MS (ESI): mass calcd. for C₂₅H₂₈ClN₃O₂, 437.97; m/z found, 438.2 [M+H]⁺.

### Example 90

### [2-(3-Chloro-benzoyl)-1,2,3,4-tetrahydro-isoquinolin-6-yl]-(octahydro-pyrido[1,2-a]pyrazin-2-yl)-methanone

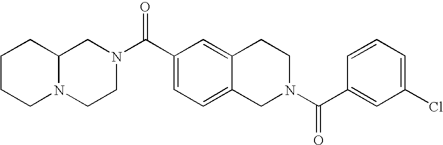

MS (ESI): mass calcd. for C₂₅H₂₈ClN₃O₂, 437.97; m/z found, 438.2 [M+H]⁺.

### Example 91

### [2-(4-Chloro-benzoyl)-1,2,3,4-tetrahydro-isoquinolin-6-yl]-(octahydro-pyrido[1,2-a]pyrazin-2-yl)-methanone

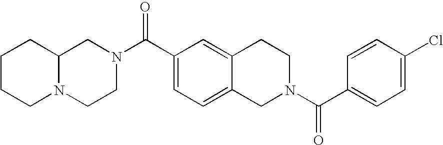

MS (ESI): mass calcd. for C₂₅H₂₈ClN₃O₂, 437.97; m/z found, 438.2 [M+H]⁺.

### Example 92

### [2-(2-Methoxy-benzoyl)-1,2,3,4-tetrahydro-isoquinolin-6-yl]-(octahydro-pyrido[1,2-a]pyrazin-2-yl)-methanone

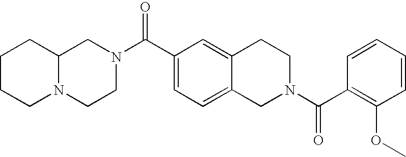

MS (ESI): mass calcd. for C₂₆H₃₁N₃O₃, 433.56; m/z found, 434.3 [M+H]⁺.

### Example 93

### [2-(3-Methoxy-benzoyl)-1,2,3,4-tetrahydro-isoquinolin-6-yl]-(octahydro-pyrido[1,2-a]pyrazin-2-yl)-methanone

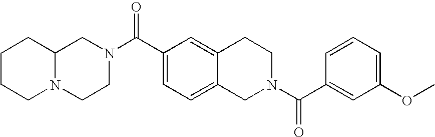

MS (ESI): mass calcd. for C₂₆H₃₁N₃O₃, 433.56; m/z found, 434.2 [M+H]⁺.

### Example 94

### 3-[6-(Octahydro-pyrido[1,2-a]pyrazine-2-carbonyl)-3,4-dihydro-1H-isoquinoline-2-carbonyl]-benzonitrile

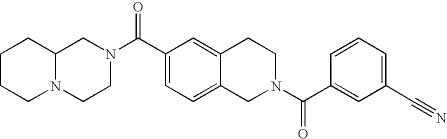

MS (ESI): mass calcd. for C₂₆H₂₈N₄O₂, 428.54; m/z found, 429.3 [M+H]⁺.

### Example 95

### 4-[6-(Octahydro-pyrido[1,2-a]pyrazine-2-carbonyl)-3,4-dihydro-1H-isoquinoline-2-carbonyl]-benzonitrile

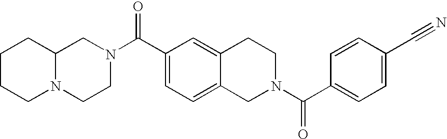

MS (ESI): mass calcd. for C₂₆H₂₈N₄O₂, 428.54; m/z found, 429.3 [M+H]⁺.

### Example 96

### [2-(2-Methyl-benzoyl)-1,2,3,4-tetrahydro-isoquinolin-6-yl]-(octahydro-pyrido[1,2-a]pyrazin-2-yl)-methanone

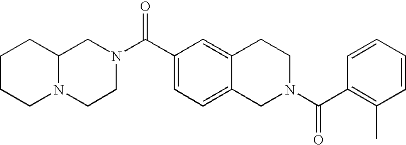

MS (ESI): mass calcd. for C₂₆H₃₁N₃O₂, 417.56; m/z found, 418.2 [M+H]⁺.

### Example 97

### [2-(4-Methyl-benzoyl)-1,2,3,4-tetrahydro-isoquinolin-6-yl]-(octahydro-pyrido[1,2-a]pyrazin-2-yl)-methanone

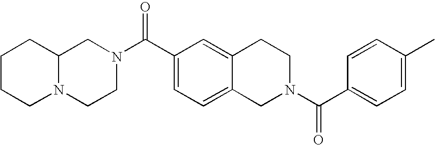

MS (ESI): mass calcd. for C₂₆H₃₁N₃O₂, 417.56; m/z found, 418.2 [M+H]⁺.

### Example 98

### [2-(4-tert-Butyl-benzoyl)-1,2,3,4-tetrahydro-isoquinolin-6-yl]-(octahydro-pyrido[1,2-a]pyrazin-2-yl)-methanone

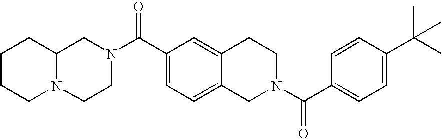

MS (ESI): mass calcd. for C₂₉H₃₇N₃O₂, 459.64; m/z found, 460.3 [M+H]⁺.

### Example 99

### (2-Benzoyl-1,2,3,4-tetrahydro-isoquinolin-6-yl)-(octahydro-pyrido[1,2-a]pyrazin-2-yl)-methanone

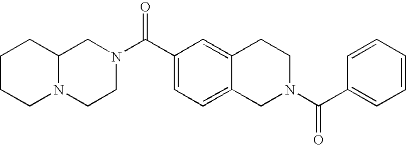

MS (ESI): mass calcd. for C₂₅H₂₉N₃O₂, 403.53; m/z found, 404.2 [M+H]⁺.

### Example 100

### (4-Cyclobutyl-piperazin-1-yl)-(2-ethanesulfonyl-1,2,3,4-tetrahydro-isoquinolin-6-yl)-methanone

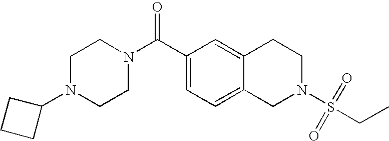

MS (ESI): mass calcd. for C₂₀H₂₉N₃O₃S, 391.19; m/z found, 392.2 [M+H]⁺. ¹H NMR (CDCl₃): 7.22-7.20 (m, 2H), 7.11 (d, J=7.7, 1H), 4.52 (s, 2H), 3.79 (bs, 2H), 3.61 (t, J=5.9, 2H), 3.44 (bs, 2H), 3.03 (q, J=7.4, 2H), 2.97 (t, J=5.8, 2H), 2.75 (p, J=7.4, 1H), 2.40 (bs, 2H), 2.26 (bs, 2H), 2.07-2.02 (m, 2H), 1.91-1.84 (m, 2H), 1.76-1.67 (m, 2H), 1.37 (t, J=7.4, 3H).

### Example 101

### (4-Cyclobutyl-piperazin-1-yl)-[2-(propane-1-sulfonyl)-1,2,3,4-tetrahydro-isoquinolin-6-yl]-methanone

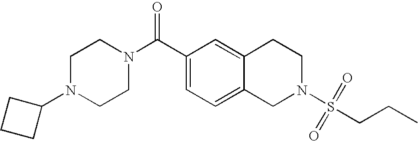

MS (ESI): mass calcd. for C₂₁H₃₁N₃O₃S, 405.21; m/z found, 406.2 [M+H]⁺.

### Example 102

### (4-Cyclobutyl-piperazin-1-yl)-[2-(propane-2-sulfonyl)-1,2,3,4-tetrahydro-isoquinolin-6-yl]-methanone

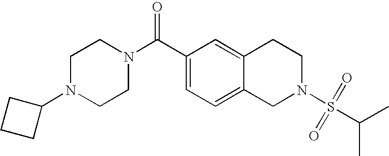

MS (ESI): mass calcd. for C₂₁H₃₁N₃O₃S, 405.21; m/z found, 406.2 [M+H]⁺.

### Example 103

### (2-Benzenesulfonyl-1,2,3,4-tetrahydro-isoquinolin-6-yl)-(4-cyclobutyl-piperazin-1-yl)-methanone

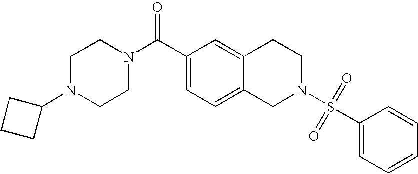

MS (ESI): mass calcd. for C₂₄H₂₉N₃O₃S, 439.19; m/z found, 440.2 [M+H]⁺. ¹H NMR (CDCl₃): 7.85 (d, J=7.5, 2H), 7.61 (t, J=7.5, 1H), 7.55 (t, J=7.5, 2H), 7.16 (d, J=7.8, 1H), 7.15 (s, 1H), 7.06 (d, J=7.8, 1H), 4.28 (s, 2H), 3.77 (bs, 2H), 3.41-3.37 (m, 4H), 2.95 (t, J=5.8, 2H), 2.73 (p, J=7.9, 1H), 2.38 (bs, 2H), 2.24 (bs, 2H), 2.06-2.01 (m, 2H), 1.90-1.83 (m, 2H), 1.76-1.67 (m, 2H).

### Example 104

### (4-Cyclobutyl-piperazin-1-yl)-[2-(4-fluoro-benzenesulfonyl)-1,2,3,4-tetrahydro-isoquinolin-6-yl]-methanone

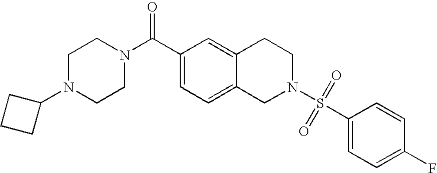

MS (ESI): mass calcd. for C₂₄H₂₈FN₃O₃S, 457.18; m/z found, 458.2 [M+H]⁺. ¹H NMR (CDCl₃): 7.87-7.85 (m, 2H), 7.22 (t, J=8.5, 2H), 7.17 (d, J=7.9, 1H), 7.16 (s, 1H), 7.07 (d, J=7.9, 1H), 4.28 (s, 2H), 3.77 (bs, 2H), 3.41-3.38 (m, 4H), 2.95 (t, J=5.8, 2H), 2.74 (p, J=7.9, 1H), 2.38 (bs, 2H), 2.24 (bs, 2H), 2.06-2.02 (m, 2H), 1.90-1.83 (m, 2H), 1.76-1.70 (m, 2H).

### Example 105

### [2-(4-Chloro-benzoyl)-1,2,3,4-tetrahydro-isoquinolin-6-yl]-(4-cyclobutyl-piperazin-1-yl)-methanone

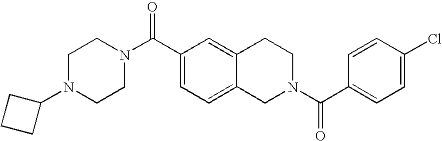

To a solution of (4-cyclobutyl-piperazin-1-yl)-(1,2,3,4-tetrahydro-isoquinolin-6-yl)-methanone (90 mg, 0.3 mmol) in DCM (5 mL) was added EDC (110 mg, 0.75 mmol), HOBt (110 mg, 0.81 mmol), and 4-chlorobenzoic acid (110 g, 0.70 mmol). After 24 h, the mixture was diluted with 1 N NaOH (10 mL) and extracted with DCM (2×10 mL). The combined organic layers were washed with brine, dried and concentrated. The resulting yellow oil was purified by reverse phase HPLC to provide 54 mg (41%) of the title compound as a white solid. MS (ESI): mass calcd. for C₂₅H₂₈ClN₃O₂, 437.19; m/z found, 438.2 [M+H]⁺. ¹H NMR (CDCl₃; mixture of rotamers): 7.43-7.39 (m, 4H), 7.26-7.23 (m, 3H), 4.88 (bs, 1.2H), 4.58 (bs, 0.8H), 3.98 (bs, 0.8H), 3.78 (bs, 2H), 3.64 (bs, 1.2H), 3.44 (bs, 2H), 2.98-2.90 (m, 2H), 2.75 (p, J=7.9, 1H), 2.39 (bs, 2H), 2.26 (bs, 2H), 2.07-2.01 (m, 2H), 1.91-1.83 (m, 2H), 1.76-1.66 (m, 2H).

The compounds from Example 106 to Example 148 were prepared using methods analogous to those described for Example 105.

### Example 106

### (4-Isopropyl-piperazin-1-yl)-[2-(thiophene-3-carbonyl)-1,2,34-tetrahydro-isoquinolin-6-yl]-methanone

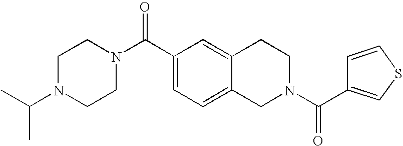

MS (ESI): mass calcd. for C₂₂H₂₇N₃O₂S, 397.18; m/z found, 398.2 [M+H]⁺.

### Example 107

### [2-(4-Hydroxy-benzoyl)-1,2,3,4-tetrahydro-isoquinolin-6-yl]-(4-isopropyl-piperazin-1-yl)-methanone

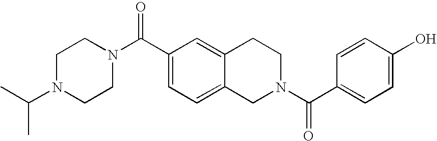

MS (ESI): mass calcd. for C₂₄H₂₉N₃O₃, 407.22; m/z found, 408.2 [M+H]⁺.

### Example 108

### (4-Isopropyl-piperazin-1-yl)-[2-(4-methoxy-benzoyl)-1,2,3,4-tetrahydro-isoquinolin-6-yl]-methanone

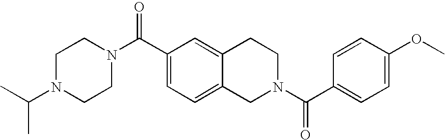

MS (ESI): mass calcd. for C₂₅H₃₁N₃O₃, 421.24; m/z found, 422.3 [M+H]⁺.

### Example 109

### (4-Isopropyl-piperazin-1-yl)-[2-(4-methyl-benzoyl)-1,2,3,4-tetrahydro-isoquinolin-6-yl]-methanone

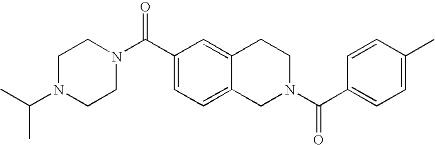

MS (ESI): mass calcd. for C₂₅H₃₁N₃O₂, 405.24; m/z found, 406.3 [M+H]⁺.

### Example 110

### [2-(4-Chloro-benzoyl)-1,2,3,4-tetrahydro-isoquinolin-6-yl]-(4-isopropyl-piperazin-1-yl)-methanone

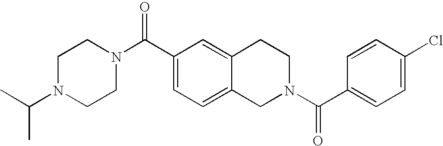

MS (ESI): mass calcd. for C₂₄H₂₈ClN₃O₂, 425.19; m/z found, 426.2 [M+H]⁺.

### Example 111

### [2-(3,4-Dichloro-benzoyl)-1,2,3,4-tetrahydro-isoquinolin-6-yl]-(4-isopropyl-piperazin-1-yl)-methanone

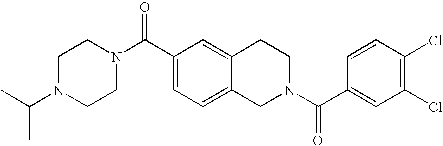

MS (ESI): mass calcd. for C₂₄H₂₇Cl₂N₃O₂, 459.15; m/z found, 460.1 [M+H]⁺.

### Example 112

### (4-Cyclobutyl-piperazin-1-yl)-[2-(4-methoxy-benzoyl)-1,2,3,4-tetrahydro-isoquinolin-6-yl]-methanone

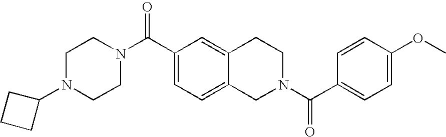

MS (ESI): mass calcd. for C₂₆H₃₁N₃O₃, 433.24; m/z found, 434.2 [M+H]⁺.

### Example 113

### (4-Cyclobutyl-piperazin-1-yl)-[2-(4-methyl-benzoyl)-1,2,3,4-tetrahydro-isoquinolin-6-yl]-methanone

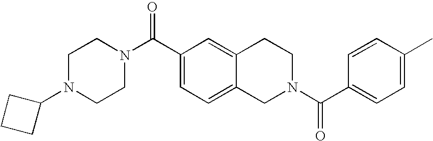

MS (ESI): mass calcd. for C₂₆H₃₁N₃O₂, 417.24; m/z found, 418.3 [M+H]⁺.

### Example 114

### (4-Cyclobutyl-piperazin-1-yl)-[2-(3,4-dichloro-benzoyl)-1,2,3,4-tetrahydro-isoquinolin-6-yl]-methanone

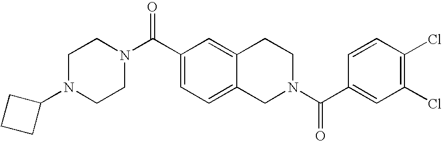

MS (ESI): mass calcd. for C₂₅H₂₇Cl₂N₃O₂, 471.15; m/z found, 472.2 [M+H]⁺.

### Example 115

### (4-Cyclobutyl-piperazin-1-yl)-[2-(thiophene-3-carbonyl)-1,2,3,4-tetrahydro-isoquinolin-6-yl]-methanone

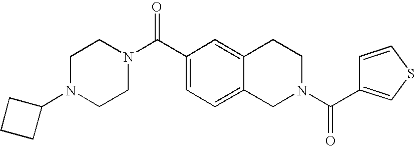

MS (ESI): mass calcd. for C₂₃H₂₇N₃O₂S, 409.18; m/z found, 410.2 [M+H]⁺. ¹H NMR (CDCl₃): 7.59 (dd, J=2.9, 1.1, 1H), 7.37 (dd, J=5.0, 2.9, 1H), 7.24 (dd, J=5.0, 1.1, 1H), 7.23-7.21 (m, 3H), 4.84 (bs, 2H), 3.99-3.79 (m, 4H), 3.44 (bs, 2H), 2.94 (bs, 2H), 2.75 (p, J=7.9, 1H), 2.40 (bs, 2H), 2.26 (bs, 2H), 2.07-2.01 (m, 2H), 1.91-1.83 (m, 2H), 1.77-1.64 (m, 2H).

### Example 116

### [6-(4-Cyclobutyl-piperazine-1-carbonyl)-3,4-dihydro-1H-isoquinolin-2-yl]-(3-dimethylamino-phenyl)-methanone

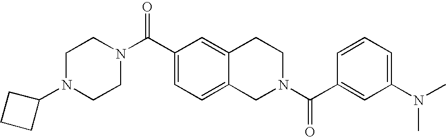

MS (ESI): mass calcd. for C₂₇H₃₄N₄O₂, 446.27; m/z found, 447.3 [M+H]⁺.

### Example 117

### [6-(4-Cyclobutyl-piperazine-1-carbonyl)-3,4-dihydro-1H-isoquinolin-2-yl]-(4-dimethylamino-phenyl)-methanone

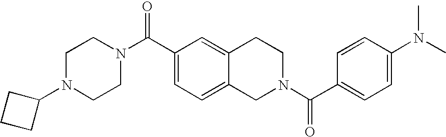

MS (ESI): mass calcd. for C₂₇H₃₄N₄O₂, 446.27; m/z found, 447.3 [M+H]⁺.

### Example 118

### [6-(4-Cyclobutyl-piperazine-1-carbonyl)-3,4-dihydro-1H-isoquinolin-2-yl]-(2,4-dichloro-phenyl)-methanone

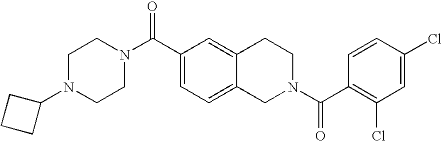

MS (ESI): mass calcd. for C₂₅H₂₇Cl₂N₃O₂, 471.15; m/z found, 472.2 [M+H]⁺.

### Example 119

### (3-Chloro-phenyl)-[6-(4-cyclobutyl-piperazine-1-carbonyl)-3,4-dihydro-1H-isoquinolin-2-yl]-methanone

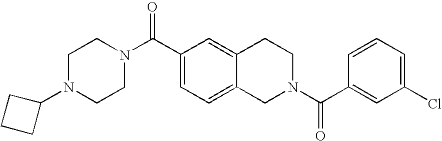

MS (ESI): mass calcd. for C₂₅H₂₈ClN₃O₂, 437.19; m/z found, 438.2 [M+H]⁺.

### Example 120

### [6-(4-Cyclobutyl-piperazine-1-carbonyl)-3,4-dihydro-1H-isoquinolin-2-yl]-m-tolyl-methanone

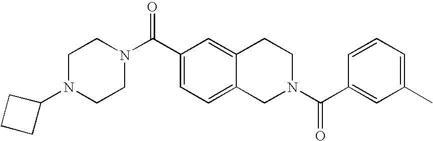

MS (ESI): mass calcd. for C₂₆H₃₁N₃O₂, 417.24; m/z found, 418.3 [M+H]⁺.

### Example 121

### [6-(4-Cyclobutyl-piperazine-1-carbonyl)-3,4-dihydro-1H-isoquinolin-2-yl]-(3-nitro-phenyl)-methanone

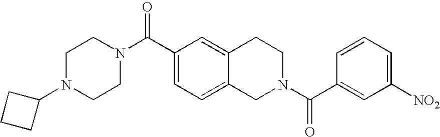

MS (ESI): mass calcd. for C₂₅H₂₈N₄O₄, 448.21; m/z found, 449.2 [M+H]⁺.

### Example 122

### [6-(4-Cyclobutyl-piperazine-1-carbonyl)-3,4-dihydro-1H-isoquinolin-2-yl]-(4-nitro-phenyl)-methanone

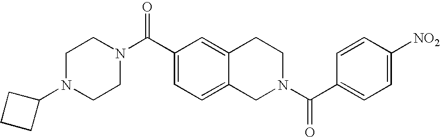

MS (ESI): mass calcd. for C₂₅H₂₈N₄O₄, 448.21; m/z found, 449.2 [M+H]⁺.

### Example 123

### [6-(4-Cyclobutyl-piperazine-1-carbonyl)-3,4-dihydro-1H-isoquinolin-2-yl]-(4-hydroxy-phenyl)-methanone

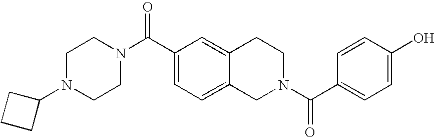

MS (ESI): mass calcd. for C₂₅H₂₉N₃O₃, 419.22; m/z found, 420.2 [M+H]⁺.

### Example 124

### (4-Cyclobutyl-piperazin-1-yl)-[2-(4-fluoro-3-hydroxy-benzoyl)-1,2,34-tetrahydro-isoquinolin-6-yl]-methanone

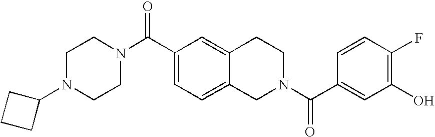

MS (ESI): mass calcd. for C₂₅H₂₈FN₃O₃, 437.52; m/z found, 438.2 [M+H]⁺.

### Example 125

### (4-Cyclobutyl-piperazin-1-yl)-[2-(4-fluoro-benzoyl)-1,2,3,4-tetrahydro-isoquinolin-6-yl]-methanone

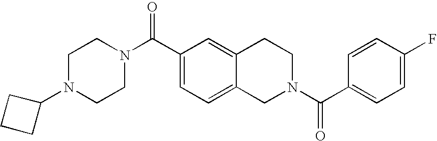

MS (ESI): mass calcd. for C₂₅H₂₈FN₃O₂, 421.52; m/z found, 422.2 [M+H]⁺. ¹H NMR (CDCl₃): 7.47 (dd, J=8.5, 5.6, 2H), 7.33-6.95 (m, 3H), 7.13 (t, J=8.5, 2H), 4.88-4.61 (m, 2H), 4.02-3.45 (m, 6H), 2.93 (bs, 2H), 2.75 (p, J=8.0, 1H), 2.40 (bs, 2H), 2.26 (bs, 2H), 2.07-2.01 (m, 2H), 1.92-1.83 (m, 2H), 1.77-1.65 (m, 2H).

### Example 126

### (4-Cyclobutyl-piperazin-1-yl)-[2-(2,4-difluoro-benzoyl)-1,2,3,4-tetrahydro-isoquinolin-6-yl]-methanone

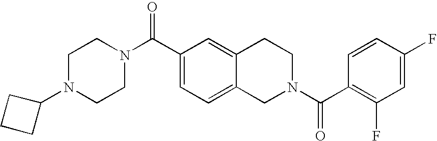

MS (ESI): mass calcd. for C₂₅H₂₇F₂N₃O₂, 439.51; m/z found, 440.2 [M+H]⁺. ¹H NMR (CDCl₃): 7.47-7.39 (m, 1H), 7.26-7.17 (m, 3H), 7.01-6.94 (m, 1H), 6.92-6.87 (m, 1H), 4.93-4.51 (m, 2H), 4.00-3.45 (m, 6H), 3.00-2.89 (m, 2H), 2.75 (p, J=7.8, 1H), 2.40 (bs, 2H), 2.27 (bs, 2H), 2.05-2.01 (m, 2H), 1.90-1.83 (m, 2H), 1.75-1.68 (m, 2H).

### Example 127

### (4-Cyclobutyl-piperazin-1-yl)-[2-(3-fluoro-4-methyl-benzoyl)-1,2,3,4-tetrahydro-isoquinolin-6-yl]-methanone

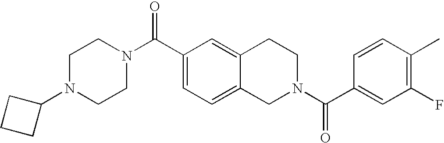

MS (ESI): mass calcd. for C₂₆H₃₀FN₃O₂, 435.55; m/z found, 436.2 [M+H]⁺. ¹H NMR (CDCl₃): 7.28-7.23 (m, 4H), 7.14-7.11 (m, 2H), 4.88-4.61 (m, 2H), 3.97-3.45 (m, 6H), 2.96-2.87 (m, 2H), 2.75 (p, J=7.8, 1H), 2.40 (bs, 2H), 2.32 (s, 3H), 2.27 (bs, 2H), 2.07-2.02 (m, 2H), 1.93-1.83 (m, 2H), 1.77-1.66 (m, 2H).

### Example 128

### [2-(3-Chloro-4-fluoro-benzoyl)-1,2,3,4-tetrahydro-isoquinolin-6-yl]-(4-cyclobutyl-piperazin-1-yl)-methanone

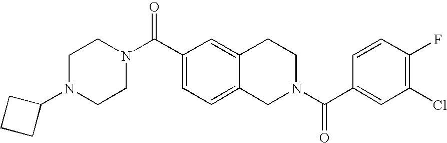

MS (ESI): mass calcd. for C₂₅H₂₇ClFN₃O₂, 455.96; m/z found, 459.2 [M+H]⁺. ¹H NMR (CDCl₃): 7.54 (d, J=6.8, 1H), 7.35 (bs, 1H), 7.28-7.19 (m 4H), 4.87-4.60 (m, 2H), 3.96-3.45 (m, 6H), 2.95-2.91 (m, 2H), 2.75 (p, J=7.6, 1H), 2.39 (bs, 2H), 2.26 (bs, 2H), 2.05-2.01 (m, 2H), 1.92-1.83 (m, 2H), 1.75-1.68 (m, 2H).

### Example 129

### 1-[6-(4-Cyclobutyl-piperazine-1-carbonyl)-3,4-dihydro-1H-isoquinolin-2-yl]-2-phenyl-ethanone

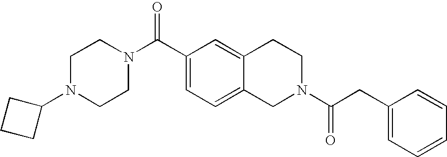

MS (ESI): mass calcd. for C₂₆H₃₁N₃O₂, 417.56; m/z found, 418.3 [M+H]⁺. ¹H NMR (CDCl₃): 7.35-7.24 (m, 5H), 7.22-7.00 (m, 3H), 4.78-4.61 (m, 2H), 3.87-3.42 (m, 6H), 3.78 (s, 2H), 2.88-2.66 (m, 3H), 2.38 (bs, 2H), 2.25 (bs, 2H), 2.06-2.01 (m, 2H), 1.92-1.83 (m, 2H), 1.74-1.67 (m, 2H).

### Example 130

### 1-[6-(4-Cyclobutyl-piperazine-1-carbonyl)-3,4-dihydro-1H-isoquinolin-2-yl]-2-(4-fluoro-phenyl)-ethanone

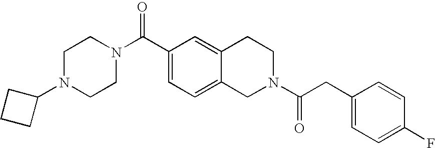

MS (ESI): mass calcd. for C₂₆H₃₀FN₃O₂, 435.55; m/z found, 436.2 [M+H]⁺.

### Example 131

### [2-(4-tert-Butyl-benzoyl)-1,2,3,4-tetrahydro-isoquinolin-6-yl]-(4-cyclobutyl-piperazin-1-yl)-methanone

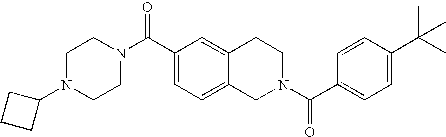

MS (ESI): mass calcd. for C₂₉H₃₇N₃O₂, 459.64; m/z found, 460.3 [M+H]⁺.

### Example 132

### (4-Cyclobutyl-piperazin-1-yl)-[2-(4-cyclohexyl-benzoyl)-1,2,3,4-tetrahydro-isoquinolin-6-yl]-methanone

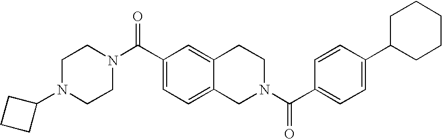

MS (ESI): mass calcd. for C₃₁H₃₉N₃O₂, 485.68; m/z found, 486.3 [M+H]⁺.

### Example 133

### (4-Chloro-phenyl)-[6-(4-cyclobutyl-[1,4]diazepane-1-carbonyl)-3,4-dihydro-1H-isoquinolin-2-yl]-methanone

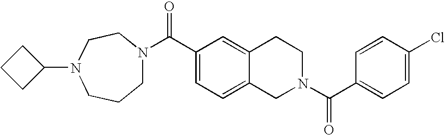

MS (ESI): mass calcd. for C₂₆H₃₀ClN₃O₂, 451.20; m/z found, 452.2 [M+H]⁺.

### Example 134

### [6-(4-Cyclobutyl-[1,4]diazepane-1-carbonyl)-3,4-dihydro-1H-isoquinolin-2-yl]-(4-fluoro-phenyl)-methanone

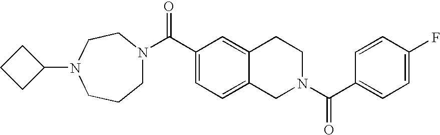

MS (ESI): mass calcd. for C₂₆H₃₀FN₃O₂, 435.23; m/z found, 436.2 [M+H]⁺.

### Example 135

### (3-Chloro-phenyl)-[6-(4-cyclobutyl-[1,4]diazepane-1-carbonyl)-3,4-dihydro-1H-isoquinolin-2-yl]-methanone

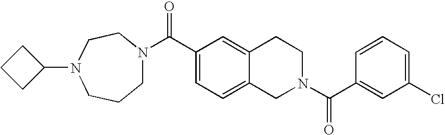

MS (ESI): mass calcd. for C₂₆H₃₀ClN₃O₂, 451.20; m/z found, 452.2 [M+H]⁺.

### Example 136

### [6-(4-Cyclobutyl-[1,4]diazepane-1-carbonyl)-3,4-dihydro-1H-isoquinolin-2-yl]-(2-fluoro-phenyl)-methanone

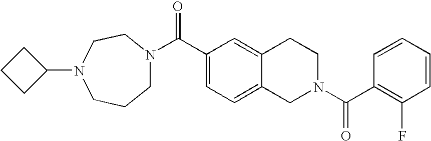

MS (ESI): mass calcd. for C₂₆H₃₀FN₃O₂, 435.23; m/z found, 436.2 [M+H]⁺.

### Example 137

### [6-(4-Cyclobutyl-[1,4]diazepane-1-carbonyl)-3,4-dihydro-1H-isoquinolin-2-yl]-(tetrahydro-furan-3-yl)-methanone

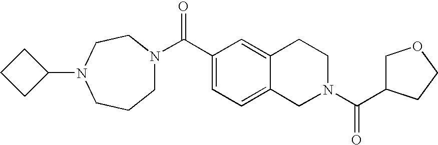

MS (ESI): mass calcd. for C₂₄H₃₃N₃O₃, 411.25; m/z found, 412.3 [M+H]⁺.

### Example 138

### [6-(4-Cyclobutyl-[1,4]diazepane-1-carbonyl)-3,4-dihydro-1H-isoquinolin-2-yl]-(tetrahydro-furan-2-yl)-methanone

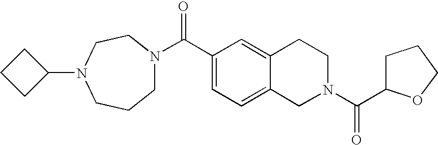

MS (ESI): mass calcd. for C₂₄H₃₃N₃O₃, 411.25; m/z found, 412.3 [M+H]⁺.

### Example 139

### 1-[6-(4-Cyclobutyl-[1,4]diazepane-1-carbonyl)-3,4-dihydro-1H-isoquinolin-2-yl]-propan-1-one

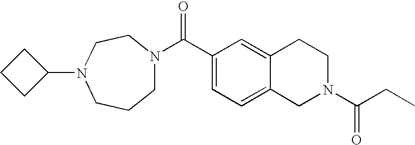

MS (ESI): mass calcd. for C₂₂H₃₁N₃O₂, 369.24; m/z found, 370.3 [M+H]⁺. ¹H NMR (CDCl₃; mixture of rotamers): 7.22-7.11 (m, 3H), 4.75 (s, 1.2H), 4.62 (s, 0.8H), 3.84 (t, J=5.9, 0.8H), 3.78-3.76 (m, 2H), 3.68 (t, J=5.9, 1.2H), 3.50-3.43 (m, 2H), 2.92-2.84 (m, 3H), 2.63-2.61 (m, 1H), 2.52-2.50 (m, 1H), 2.46-2.41 (m, 4H), 2.08-1.94 (m, 3H), 1.88-1.76 (m, 3H), 1.71-1.58 (m, 2H), 1.21-1.17 (m, 3H).

### Example 140

### [6-(4-Cyclobutyl-[1,4]diazepane-1-carbonyl)-3,4-dihydro-1H-isoquinolin-2-yl]-(4-propyl-phenyl)-methanone

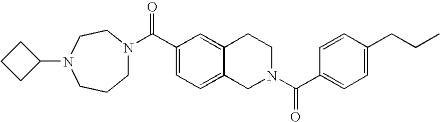

MS (ESI): mass calcd. for C₂₉H₃₇N₃O₂, 459.29; m/z found, 460.3 [M+H]⁺.

### Example 141

### [6-(4-Cyclobutyl-[1,4]diazepane-1-carbonyl)-3,4-dihydro-1H-isoquinolin-2-yl]-(4-fluoro-3-hydroxy-phenyl)-methanone

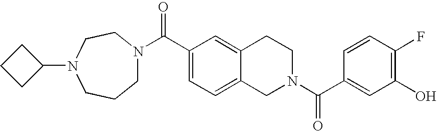

MS (ESI): mass calcd. for C₂₆H₃₀FN₃O₃, 451.23; m/z found, 452.3 [M+H]⁺.

### Example 142

### [6-(4-Cyclobutyl-[1,4]diazepane-1-carbonyl)-3,4-dihydro-1H-isoquinolin-2-yl]-(3-fluoro-4-methyl-phenyl)-methanone

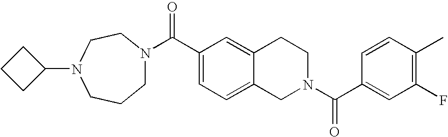

MS (ESI): mass calcd. for C₂₇H₃₂FN₃O₂, 449.25; m/z found, 450.3 [M+H]⁺.

### Example 143

### [6-(4-Cyclobutyl-[1,4]diazepane-1-carbonyl)-3,4-dihydro-1H-isoquinolin-2-yl]-(2,4-dichloro-phenyl)-methanone

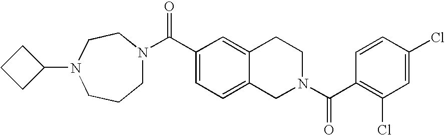

MS (ESI): mass calcd. for C₂₆H₂₉Cl₂N₃O₂, 485.16; m/z found, 486.2 [M+H]⁺.

### Example 144

### [6-(4-Cyclobutyl-[1,4]diazepane-1-carbonyl)-3,4-dihydro-1H-isoquinolin-2-yl]-(2,4-difluoro-phenyl)-methanone

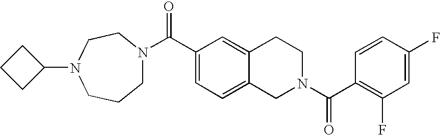

MS (ESI): mass calcd. for C₂₆H₂₉F₂N₃O₂, 453.22; m/z found, 454.3 [M+H]⁺.

### Example 145

### (3-Chloro-4-fluoro-phenyl)-[6-(4-cyclobutyl-[1,4]diazepane-1-carbonyl)-3,4-dihydro-1H-isoquinolin-2-yl]-methanone

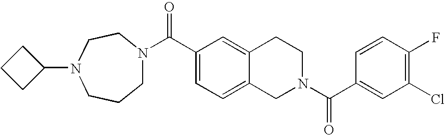

MS (ESI): mass calcd. for C₂₆H₂₉ClFN₃O₂, 469.19; m/z found, 470.2 [M+H]⁺.

### Example 146

### [6-(4-Cyclobutyl-[1,4]diazepane-1-carbonyl)-3,4-dihydro-1H-isoquinolin-2-yl]-(3-methoxy-cyclohexyl)-methanone

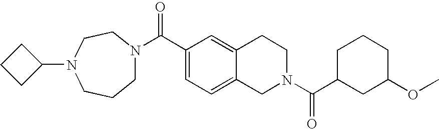

MS (ESI): mass calcd. for C₂₇H₃₉N₃O₃, 453.30; m/z found, 454.3 [M+H]⁺.

### Example 147

### trans-[6-(4-Cyclobutyl-[1,4]diazepane-1-carbonyl)-3,4-dihydro-1H-isoquinolin-2-yl]-(4-methoxy-cyclohexyl)-methanone (racemic mixture)

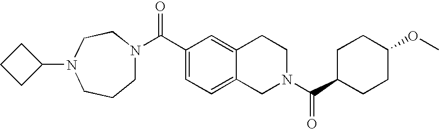

MS (ESI): mass calcd. for C₂₇H₃₉N₃O₃, 453.30; m/z found, 454.3 [M+H]⁺.

### Example 148

### cis-[6-(4-Cyclobutyl-[1,4]diazepane-1-carbonyl)-3,4-dihydro-1H-isoquinolin-2-yl]-(4-methoxy-cyclohexyl)-methanone (racemic mixture)

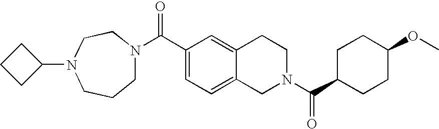

MS (ESI): mass calcd. for C₂₇H₃₉N₃O₃, 453.30; m/z found, 454.3 [M+H]⁺.

### Example 149

### [2-(1-Isopropyl-piperidine-4-carbonyl)-1,2,3,4-tetrahydro-isoquinolin-6-yl]-morpholin-4-yl-methanone

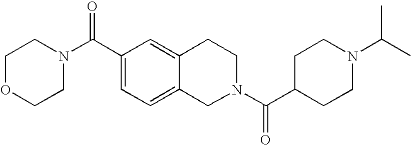

Step A: Potassium 1-isopropyl-piperidine-4-carboxylate. A solution of methyl isonipecotate (19.3 mL, 143 mmol), acetone (21.0 mL, 285 mmol), and acetic acid (15.6 mL, 285 mmol) in DCE (500 mL) was stirred for 3 h. NaBH(OAc)₃ (45.4 g, 214 mmol) was added and the solution was stirred at rt for 18 h. The mixture was diluted with 1 N NaOH (300 mL) and extracted with DCM (3×300 mL). The combined organic layers were washed with brine, dried and concentrated to give 1-isopropyl-piperidine-4-carboxylic acid methyl ester, which was carried to the next step without further purification. MS (ESI): mass calcd. for C₁₀H₁₆NO₂, 185.14; m/z found, 186.2 [M+H]⁺. The crude 1-isopropyl-piperidine-4-carboxylic acid methyl ester was dissolved in i-PrOH (500 mL) and treated with 2 N KOH (86 mL). The solution was heated at 80° C. for 20 h and then concentrated leaving a tan solid (15.1 g, 51% over 2 steps), which was used in subsequent steps without further purification. MS (ESI): mass calcd. for C₉H₁₆KNO₂, 209.08; m/z found, 172.2 [M-K+H]⁺.

Step B: 2-(1-Isopropyl-piperidine-4-carbonyl)-1,2,3,4-tetrahydro-isoquinolin-6-yl]-morpholin-4-yl-methanone. To a solution of morpholin-4-yl-(1,2,3,4-tetrahydro-isoquinolin-6-yl)-methanone (74 mg, 0.30) in DMF (3 mL) was added potassium 1-isopropyl-piperidine-4-carboxylate (75 mg, 0.30 mmol), EDC (86 mg, 0.45 mmol), and HOBt (61 mg, 0.45 mmol). After 20 h, the mixture was diluted with satd. aq. NaHCO₃ (3 mL) and extracted with DCM (3×10 mL). The combined organic layers were washed with brine, dried and concentrated. The resulting residue was purified by FCC to provide the title compound as a colorless oil (15.8 mg, 13%). MS (ESI): mass calcd. for C₂₃H₃₃N₃O₃, 399.25; m/z found, 400.3 [M+H]⁺. ¹H NMR (CDCl₃; mixture of rotamers): 7.23-7.15 (m, 3H), 4.74 (s, 1.2H), 4.67 (s, 0.8H), 3.84-3.60 (m, 8H), 3.46 (bs, 2H), 2.97-2.91 (m, 3.2H), 2.87-2.84 (m, 0.8H), 2.73 (sept, J=6.4, 1H), 2.52 (m, 1H), 2.20 (m, 2H), 1.92-1.82 (m, 2H), 1.78-1.69 (m, 2H), 1.05 (d, J=6.4, 6H).

### Example 150

### (S)-Cyclohexyl-[6-(2-Pyrrolidin-1-ylmethyl-pyrrolidine-1-carbonyl)-3,4-dihydro-1H-isoquinolin-2-yl]-methanone

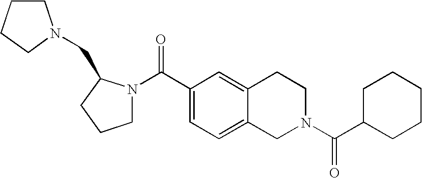

Step A: 2-Cyclohexanecarbonyl-1,2,3,4-tetrahydro-isoquinoline-6-carboxylic acid methyl ester. To a 0° C. solution of 1,2,3,4-tetrahydro-isoquinoline-6-carboxylic acid methyl ester (8.0 g, 35 mmol) in DCM (350 mL) was added TEA (9.8 mL, 70 mmol) and cyclohexanecarbonyl chloride (9.5 mL, 70 mmol). The reaction mixture was allowed to warm to rt over 16 h during which time triethylammonium chloride precipitated. This solid was removed by filtration, and the remaining solution was allowed to stand at rt for 2 h. The white precipitate that formed was collected by filtration and dried to give (8.2 g, 77%) of the title compound. MS (ESI): mass calcd. for C₁₈H₂₃NO₃, 301.17; m/z found 302.2, [M+H]⁺. ¹H NMR (CDCl₃; mixture of rotamers): 7.87-7.84 (m, 2H), 7.22-7.19 (m, 1H), 4.77 (s, 1.2H), 4.71 (s, 0.8H), 3.91 (s, 3H), 3.84 (t, J=5.7, 0.8H), 3.74 (t, J=5.7, 1.2H), 2.96 (t, J=5.7, 1.2H), 2.88 (t, J=5.7, 0.8H), 2.56 (tt, J=11.6, 3.4, 1H), 1.85-1.69 (m, 5H), 1.60-1.51 (m, 2H), 1.35-1.22 (m, 3H).

Step B: 2-Cyclohexanecarbonyl-1,2,3,4-tetrahydro-isoquinoline-6-carboxylic acid. To a solution of 2-cyclohexanecarbonyl-1,2,3,4-tetrahydro-isoquinoline-6-carboxylic acid methyl ester (8.15 g, 27.1 mmol) in i-PrOH (250 mL) was added 2 N KOH (16.2 mL, 32.5 mmol). The solution was stirred at 80° C. for 20 h, concentrated and then dissolved in water. 6 N HCl was added dropwise until the product precipitated from solution. The white solid was collected and dried under vacuum to provide 7.3 g (94%) of the title compound. MS (ESI): mass calcd. for C₁₇H₂₁NO₃, 287.15; m/z found 288.2, [M+H]⁺.

Step C: (S)-Cyclohexyl-[6-(2-pyrrolidin-1-ylmethyl-pyrrolidine-1-carbonyl)-3,4-dihydro-1H-isoquinolin-2-yl]-methanone. To a solution of 2-cyclohexanecarbonyl-1,2,3,4-tetrahydro-isoquinoline-6-carboxylic acid (0.200 g, 0.700 mol), EDC (0.208 g, 0.108 mol), and HOBt (0.146 mg, 0.108 mol) in DCM (8 mL) was added (S)-(+)-1-(2-pyrrolidinylmethyl)pyrrolidine (0.130 mg, 0.840 mmol). After 24 h, the mixture was diluted with 1 N NaOH (10 mL) and extracted with DCM (2×10 mL). The combined organic layers were washed with brine, dried and concentrated. The resulting residue was purified by reverse phase HPLC to provide 124 mg (41%) of the title compound as a white solid. MS (ESI): mass calcd. for C₂₆H₃₇N₃O₂, 423.29; m/z found 424.3 [M+H]⁺. ¹H NMR (CDCl₃; mixture of rotamers): 7.34-7.27 (m, 2H), 7.18-7.13 (m, 1H), 4.76-4.67 (m, 2H), 4.43 (bs, 0.8H), 4.04-3.38 (m, 4.2H), 2.95-2.82 (m, 2.8H), 2.69 (m, 3.2H), 2.58-2.53 (m, 1H), 2.25-1.24 (m, 20H).

The compounds from Example 151 to Example 162 were prepared using methods analogous to those described for Example 150.

### Example 151

### Cyclohexyl-{6-[4-(tetrahydro-furan-2-ylmethyl)-piperazine-1-carbonyl]-3,4-dihydro-1H-isoquinolin-2-yl}-methanone

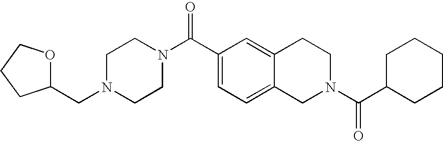

MS (ESI): mass calcd. for C₂₆H₃₇N₃O₃, 439.28; m/z found, 440.3 [M+H]⁺.

### Example 152

### Cyclohexyl-[6-(octahydro-pyrido[1,2-a]pyrazine-2-carbonyl)-3,4-dihydro-1H-isoquinolin-2-yl]-methanone

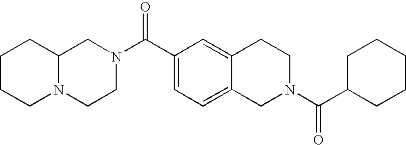

MS (ESI): mass calcd. for C₂₅H₃₅N₃O₂, 409.27; m/z found, 410.3 [M+H]⁺. ¹H NMR (CDCl₃; mixture of rotamers): 7.22-7.14 (m, 3H), 4.74 (s, 0.7H), 4.67-4.62 (m, 0.8H), 4.54-4.50 (m, 0.5H), 3.85-3.81 (m, 0.8H), 3.75-3.65 (m, 1.7H), 3.55-3.50 (m, 0.5H), 3.34-3.27 (m, 0.5H), 3.07-2.98 (m, 0.5H), 2.95-2.82 (m, 4H), 2.71-2.52 (m, 2H), 2.28-2.03 (m, 2H), 2.00-1.10 (m, 18H).

### Example 153

### Cyclohexyl-[6-(hexahydro-pyrrolo[1,2-a]pyrazine-2-carbonyl)-3,4-dihydro-1H-isoquinolin-2-yl]-methanone

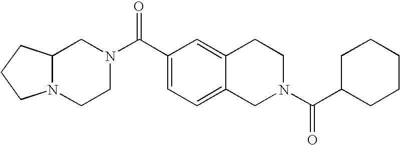

MS (ESI): mass calcd. for C₂₄H₃₃N₃O₂, 395.26; m/z found, 396.3 [M+H]⁺.

### Example 154

### Cyclohexyl-[6-(4-dimethylamino-piperidine-1-carbonyl)-3,4-dihydro-1H-isoquinolin-2-yl]-methanone

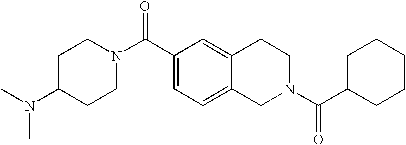

MS (ESI): mass calcd. for C₂₄H₃₅N₃O₂, 397.27; m/z found, 398.3 [M+H]⁺.

### Example 155

### (R)-Cyclohexyl-[6-(3-dimethylamino-pyrrolidine-1-carbonyl)-3,4-dihydro-1H-isoquinolin-2-yl]-methanone

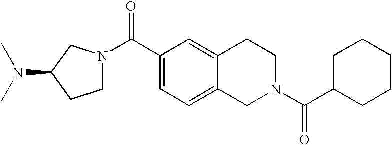

MS (ESI): mass calcd. for C₂₃H₃₃N₃O₂, 383.26; m/z found, 384.3 [M+H]⁺.

### Example 156

### (S)-Cyclohexyl-[6-(3-dimethylamino-pyrrolidine-1-carbonyl)-3,4-dihydro-1H-isoquinolin-2-yl]-methanone

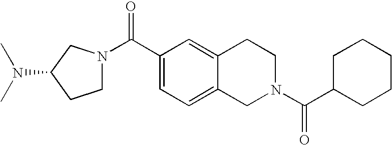

MS (ESI): mass calcd. for C₂₃H₃₃N₃O₂, 383.26; m/z found, 384.3 [M+H]⁺.

### Example 157

### [6-([1,4′]Bipiperidinyl-1′-carbonyl)-3,4-dihydro-1H-isoquinolin-2-yl]-cyclohexyl-methanone

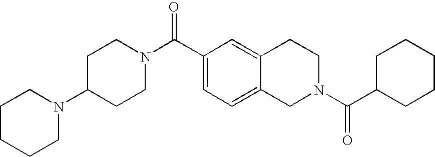

MS (ESI): mass calcd. for C₂₇H₃₉N₃O₂, 437.30; m/z found, 438.3 [M+H]⁺.

### Example 158

### Cyclohexyl-[6-(4-morpholin-4-yl-piperidine-1-carbonyl)-3,4-dihydro-1H-isoquinolin-2-yl]-methanone

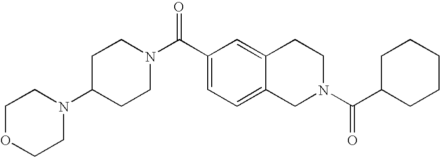

MS (ESI): mass calcd. for C₂₆H₃₇N₃O₃, 439.28; m/z found, 440.3 [M+H]⁺.

### Example 159

### Cyclohexyl-[6-(4-cyclopentyl-piperazine-1-carbonyl)-3,4-dihydro-1H-isoquinolin-2-yl]-methanone

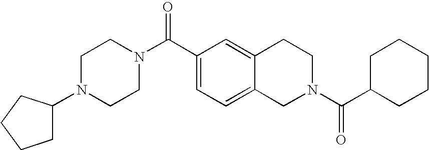

MS (ESI): mass calcd. for C₂₆H₃₇N₃O₂, 423.29; m/z found, 424.3 [M+H]⁺.

### Example 160

### Cyclohexyl-[6-(4-cyclohexyl-piperazine-1-carbonyl)-3,4-dihydro-1H-isoquinolin-2-yl]-methanone

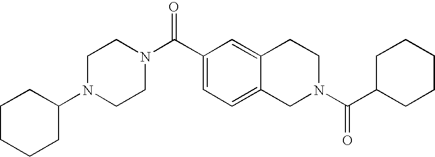

MS (ESI): mass calcd. for C₂₇H₃₉N₃O₂, 437.30; m/z found, 438.3 [M+H]⁺.

### Example 161

### 2-Cyclohexanecarbonyl-1,2,3,4-tetrahydro-isoquinoline-6-carboxylic acid methyl-(1-methyl-pyrrolidin-3-yl)-amide

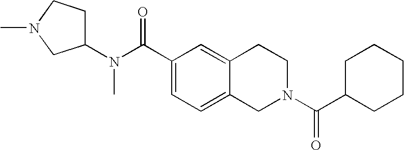

MS (ESI): mass calcd. for C₂₃H₃₃N₃O₂, 383.26; m/z found, 384.3 [M+H]⁺.

### Example 162

### Cyclohexyl-[6-(4-isopropyl-[1,4]diazepane-1-carbonyl)-3,4-dihydro-1H-isoquinolin-2-yl]-methanone

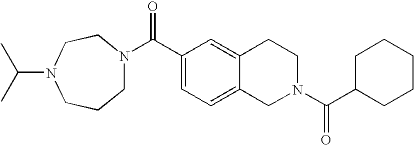

MS (ESI): mass calcd. for C₂₅H₃₇N₃O₂, 411.29; m/z found, 412.3 [M+H]⁺.

### Example 163

### (5-Cyclobutyl-hexahydro-pyrrolo[3,4-c]pyrrol-2-yl)-(2-cyclohexanecarbonyl-1,2,3,4-tetrahydro-isoquinolin-6-yl)-methanone

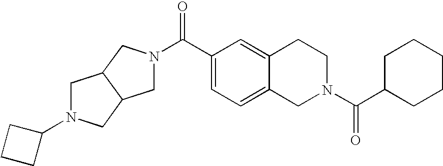

Step A: 5-(2-Cyclohexanecarbonyl-1,2,3,4-tetrahydro-isoquinoline-6-carbonyl)-hexahydro-pyrrolo[3,4-c]pyrrole-2-carboxylic acid tert-butyl ester. The title compound was prepared using methods analogous to those described in Example 150. MS (ESI): mass calcd. for C₂₈H₃₉N₃O₄, 481.29; m/z found, 482.3 [M+H]⁺.

Step B. (2-Cyclohexanecarbonyl-1,2,3,4-tetrahydro-isoquinolin-6-yl)-(hexahydro-pyrrolo[3,4-c]pyrrol-2-yl)-methanone. To a solution of 5-(2-cyclohexanecarbonyl-1,2,3,4-tetrahydro-isoquinoline-6-carbonyl)-hexahydro-pyrrolo[3,4-c]pyrrole-2-carboxylic acid tert-butyl ester (0.351 g, 0.729 mmol) in DCM (8 mL) was added TFA (4 mL). The mixture was stirred at rt for 2 h. The solution was concentrated and the resulting residue was dissolved in MeOH (20 mL) and treated with DOWEX® resin. After 2 h, the suspension was filtered and concentrated. The residue was purified by reverse phase HPLC to yield 190 mg (68%) of the title compound as a colorless gum. MS (ESI): mass calcd. for C₂₃H₃₁N₃O₂, 381.24; m/z found, 382.2 [M+H]⁺. ¹H NMR (CDCl₃; mixture of rotamers): 7.33-7.29 (m, 2H), 7.17-7.13 (m, 1H), 4.73 (s, 1.2H), 4.67 (s, 0.8H), 3.84-3.57 (m, 6H), 3.43-3.30 (m, 1H), 3.08-2.76 (m, 5H), 2.55 (tt, J=11.5, 3.3, 1H), 1.96-1.53 (m, 9H), 1.35-1.25 (m, 3H).

Step C. A solution of (2-cyclohexanecarbonyl-1,2,3,4-tetrahydro-isoquinolin-6-yl)-(hexahydro-pyrrolo[3,4-c]pyrrol-2-yl)-methanone (53 mg, 0.14 mmol), acetic acid (25 μL, 0.42 mmol), and cyclobutanone (32 μL, 0.42 mmol) in DCE (5 mL) was stirred at rt for 1 h. NaBH(OAc)₃ (89 mg, 0.42 mmol) was added and the reaction mixture was allowed to stir for 15 h. The mixture was diluted with satd. aq. NaHCO₃ (5 mL) and extracted with DCM (3×5 mL). The combined organic layers were washed with brine, dried and concentrated. The resulting residue was purified by reverse phase HPLC to provide 54 mg (89%) of the title compound as a white solid. MS (ESI): mass calcd. for C₂₇H₃₇N₃O₂, 435.29; m/z found, 436.3 [M+H]⁺. ¹H NMR (CDCl₃; mixture of rotamers): 7.33-7.27 (m, 2H), 7.18-7.13 (m, 1H), 4.74 (s, 1.2H), 4.67 (s, 0.8H), 3.84-3.49 (5H), 3.40-3.34 (m, 0.5H), 3.17-2.72 (m, 4.5H), 2.58-2.53 (m, 1H), 2.38 (q, J=8.6, 0.5H), 2.07-1.25 (m, 20.5H).

The compounds in Examples 164-165 were prepared using methods analogous to those described for Example 163.

### Example 164

### (1S,4S)-(5-Cyclobutyl-2,5-diaza-bicyclo[2.2.1]hept-2-yl)-(2-cyclohexanecarbonyl-1,2,3,4-tetrahydro-isoquinolin-6-yl)-methanone

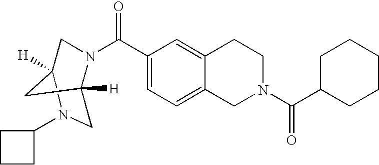

Step A: (1S,4S)-5-(2-Cyclohexanecarbonyl-1,2,3,4-tetrahydro-isoquinoline-6-carbonyl)-2,5-diaza-bicyclo[2.2.1]heptane-2-carboxylic acid tert-butyl ester. MS (ESI): mass calcd. for C₂₇H₃₇N₃O₄, 467.28; m/z found, 468.3 [M+H]⁺.

Step B: (1S,4S)-(2-Cyclohexanecarbonyl-1,2,3,4-tetrahydro-isoquinolin-6-yl)-(2,5-diaza-bicyclo[2.2.1]hept-2-yl)-methanone. MS (ESI): mass calcd. for C₂₂H₂₉N₃O₂, 367.23; m/z found, 368.2 [M+H]⁺.

Step C. MS (ESI): mass calcd. for C₂₆H₃₅N₃O₂, 421.27; m/z found, 422.3 [M+H]⁺.

### Example 165

### (1-Cyclobutyl-hexahydro-pyrrolo[3,4-b]pyrrol-5-yl)-(2-cyclohexanecarbonyl-1,2,3,4-tetrahydro-isoquinolin-6-yl)-methanone

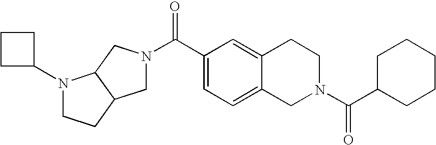

Step A: (2-Cyclohexanecarbonyl-1,2,3,4-tetrahydro-isoquinolin-6-yl)-(hexahydro-pyrrolo[3,4-b]pyrrol-5-yl)-methanone. MS (ESI): mass calcd. for C₂₈H₃₉N₃O₄, 481.29; m/z found, 482.3 [M+H]⁺.

Step B: 5-(2-Cyclohexanecarbonyl-1,2,3,4-tetrahydro-isoquinoline-6-carbonyl)-hexahydro-pyrrolo[3,4-b]pyrrole-1-carboxylic acid tert-butyl ester. MS (ESI): mass calcd. for C₂₃H₃₁N₃O₂, 381.24; m/z found, 382.3 [M+H]⁺.

Step C. MS (ESI): mass calcd. for C₂₇H₃₇N₃O₂, 435.29; m/z found, 436.3 [M+H]⁺.

### Example 166

### Cyclohexyl-(6-piperidin-1-ylmethyl-3,4-dihydro-1H-isoquinolin-2-yl)-methanone

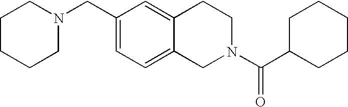

Step A: Cyclohexyl-(6-hydroxymethyl-3,4-dihydro-1H-isoquinolin-2-yl)-methanone. To a 0° C. solution of 2-cyclohexanecarbonyl-1,2,3,4-tetrahydro-isoquinoline-6-carboxylic acid (1.00 g, 3.48 mmol) in THF (35 mL) was added TEA (0.531 mL, 3.83 mmol) and isobutylchloroformate (0.501 mL, 3.83 mmol). After 2 h at 0° C., the mixture was filtered and the filtrate was reduced by half by concentration. The solution was cooled to 0° C. and treated with NaBH₄ (263 mg, 6.96 mmol). Water (15 mL) was added dropwise with stirring and the mixture was allowed to warm to rt over 16 h. The reaction was quenched with 1 N HCl (10 mL) and extracted with EtOAc (3×50 mL). The organic layers were combined, washed with brine, dried and concentrated to yield 0.79 g (83%) of a white solid, which was used in the next step without further purification. MS (ESI): mass calcd. for C₁₇H₂₃NO₂, 273.17; m/z found, 274.2 [M+H]⁺. ¹H NMR (CDCl₃; mixture of rotamers): 7.22-7.17 (m, 2H), 7.12-7.10 (m, 1H), 4.70 (s, 1.2H), 4.66 (bs, 2.8H), 3.81 (t, J=5.8, 0.8H), 3.71 (t, J=5.8, 1.2H), 2.90 (t, J=5.8, 1.2H), 2.83 (t, J=5.8, 0.8H), 2.58-2.52 (m, 1H), 1.81-1.69 (m, 5H), 1.60-1.49 (m, 2H), 1.35-1.26 (m, 3H).

Step B: 2-Cyclohexanecarbonyl-1,2,3,4-tetrahydro-isoquinoline-6-carbaldehyde. A solution of cyclohexyl-(6-hydroxymethyl-3,4-dihydro-1H-isoquinolin-2-yl)-methanone (0.750 g, 2.75 mmol) in THF/CHCl₃ (2:1) was treated with MnO₂ (1.19 g, 13.7 mmol) and the resulting mixture was heated at 60° C. for 16 h. The mixture was filtered through a pad of diatomaceous earth and the filtrate was concentrated to yield 0.71 g (95%) of a pale yellow gum. This material was used in the next reaction without further purification. MS (ESI): mass calcd. for C₁₇H₂₁NO₂, 271.16; m/z found, 272.2 [M+H]⁺.

Step C: Cyclohexyl-(6-piperidin-1-ylmethyl-3,4-dihydro-1H-isoquinolin-2-yl)-methanone. A solution of 2-cyclohexanecarbonyl-1,2,3,4-tetrahydro-isoquinoline-6-carbaldehyde (115 mg, 0.424 mmol), piperidine (51 μL, 0.51 mmol), and acetic acid (48 μL, 0.85 mmol) in DCE (4 mL) was stirred at rt for 2 h. NaBH(OAc)₃ (180 mg, 0.85 mmol) was added and the mixture was allowed to stir for 20 h. The reaction was diluted with satd. aq. NaHCO₃ (5 mL) and extracted with DCM (3×5 mL). The combined organic layers were washed with brine, dried and concentrated. The resulting yellow gum was purified by reverse phase HPLC yielding 53 mg (34%) of a pale yellow oil. MS (ESI): mass calcd. for C₂₂H₃₂N₂O, 340.25; m/z found, 341.3 [M+H]⁺. ¹H NMR (CDCl₃; mixture of rotamers): 7.17-7.05 (m, 3H), 4.70 (s, 1.15H), 4.64 (s, 0.85H), 3.81 (t, J=5.8, 0.85H), 3.71 (t, J=5.8, 1.15H), 3.42 (s, 2H), 2.90 (t, J=5.8, 1.15H), 2.82 (t, J=5.8, 0.85H), 2.59-2.52 (m, 1H), 2.36 (bs, 4H), 1.84-1.71 (m, 5H), 1.60-1.54 (m, 6H), 1.46-1.40 (m, 2H), 1.33-1.26 (m, 3H).

The compounds from Example 167 to Example 171 were prepared using methods analogous to those described for Example 166.

### Example 167

### Cyclohexyl-(6-morpholin-4-ylmethyl-3,4-dihydro-1H-isoquinolin-2-yl)-methanone

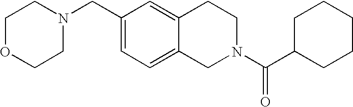

MS (ESI): mass calcd. for C₂₁H₃₀N₂O₂, 342.23; m/z found, 343.3 [M+H]⁺.

### Example 168

### Cyclohexyl-[6-(octahydro-pyrido[1,2-a]pyrazin-2-ylmethyl)-3,4-dihydro-1H-isoquinolin-2-yl]-methanone

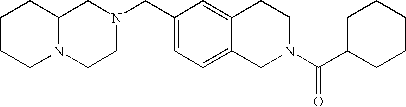

MS (ESI): mass calcd. for C₂₅H₃₇N₃O, 395.29; m/z found, 396.3 [M+H]⁺. ¹H NMR (CDCl₃; mixture of rotamers): 7.16-7.06 (m, 3H), 4.70 (s, 1.2H), 4.64 (s, 0.8H), 3.81 (t, J=5.8, 0.8H), 3.71 (t, J=5.8, 1.2H), 3.45-3.43 (m, 2H), 2.90-2.88 (m, 1.2H), 2.83-2.78 (m, 2.8H), 2.72-2.66 (m, 2H), 2.57-2.53 (m, 1H), 2.33-2.23 (m, 2H), 2.06-1.97 (m, 2H), 1.87-1.69 (m, 7H), 1.63-1.46 (m, 5H), 1.34-1.16 (m, 5H).

### Example 169

### Cyclohexyl-[6-(2-pyrrolidin-1-ylmethyl-pyrrolidin-1-ylmethyl)-3,4-dihydro-1H-isoquinolin-2-yl]-methanone

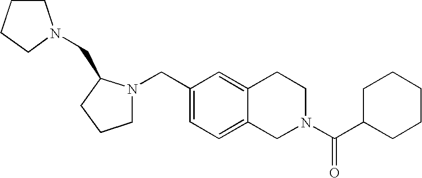

MS (ESI): mass calcd. for C₂₆H₃₉N₃O, 409.31; m/z found, 410.3 [M+H]⁺.

### Example 170

### [6-(4-Cyclobutyl-piperazin-1-ylmethyl)-3,4-dihydro-1H-isoquinolin-2-yl]-cyclohexyl-methanone

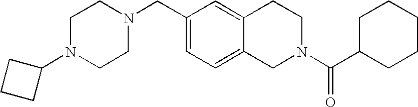

MS (ESI): mass calcd. for C₂₅H₃₇N₃O, 395.29; m/z found, 396.3 [M+H]⁺.

### Example 171

### [6-(4-Cyclobutyl-[1,4]diazepan-1-ylmethyl)-3,4-dihydro-1H-isoquinolin-2-yl]-cyclohexyl-methanone

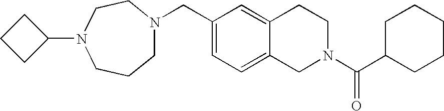

MS (ESI): mass calcd. for C₂₆H₃₉N₃O, 409.31; m/z found, 410.3 [M+H]⁺. ¹H NMR (CDCl₃; mixture of rotamers): 7.19-7.05 (m, 3H), 4.71 (s, 1.2H), 4.64 (s, 0.85), 3.82 (t, J=5.8, 0.8H), 3.71 (t, J=5.8, 1.2H), 3.59 (s, 2H), 2.94-2.88 (m, 2.2H), 2.82 (t, J=5.8, 0.8H), 2.71-2.66 (m, 4H), 2.56-2.50 (m, 5H), 2.03-1.99 (m, 2H), 1.86-1.51 (m, 13H), 1.34-1.25 (m, 3H).

### Example 172

### (2-Cyclopentyl-1,2,3,4-tetrahydro-isoquinolin-6-yl)-morpholin-4-yl-methanone

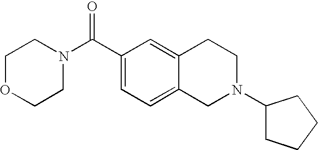

Step A: 2-Cyclopentyl-1,2,3,4-tetrahydro-isoquinoline-6-carboxylic acid isobutyric anhydride. To a 0° C. solution of potassium 2-cyclopentyl-1,2,3,4-tetrahydro-isoquinoline-6-carboxylate (5.9 g, 21 mmol) in THF/DMF (200 mL/10 mL) was added TEA (3.2 mL, 23 mmol) and isobutylchloroformate (3.2 mL, 23 mmol). The solution was stirred for 20 h while warming to rt. The reaction mixture was concentrated, diluted with brine (100 mL), and extracted with DCM (3×100 mL) to yield 6.9 g (96%) of a brown oil. This product was used in the next step without further purification. MS (ESI): mass calcd. for C₂₀H₂₇NO₄, 345.19; m/z found, 346.2. ¹H NMR (CDCl₃): 7.82-1.80 (m, 2H), 7.13 (d, J=7.9, 1H), 3.75-3.40 (m, 8H), 2.96 (t, J=5.9, 2H), 2.80 (t, J=5.9, 2H), 2.72 (p, J=8.0, 1H), 2.00-1.95 (m, 2H), 1.77-1.71 (m, 2H), 1.63-1.48 (m, 3H), 1.01 (d, J=6.7, 4H).

Step B: (2-Cyclopentyl-1,2,3,4-tetrahydro-isoquinolin-6-yl)-morpholin-4-yl-methanone. To a solution of morpholine (47 μL, 0.53 mmol) and TEA (74 μL, 0.53 mmol) in DCM (3 mL) was added 2-cyclobutyl-1,2,3,4-tetrahydro-isoquinoline-6-carboxylic acid isobutyric anhydride (120 mg, 0.35 mmol). After 20 h, the mixture was concentrated and the resulting residue was purified by reverse phase HPLC to provide 17 mg (15%) of the title compound as a white solid. MS (ESI): mass calcd. for C₁₉H₂₆N₂O₂, 314.42; m/z found, 315.2. ¹H NMR (CDCl₃): 7.15 (s, 1H), 7.13 (d, J=7.8, 1H), 7.06 (d, J=7.8, 1H), 3.81-3.38 (m, 10H), 2.92 (t, J=5.9, 2H), 2.80 (t, J=5.9, 2H), 2.70 (p, J=8.0, 1H), 2.00-1.94 (m, 2H), 1.77-1.70 (m, 2H), 1.62-1.48 (m, 4H).

The compounds from Example 173 to Example 194 were prepared using methods analogous to those described for Example 172.

### Example 173

### (2-Isopropyl-1,2,3,4-tetrahydro-isoquinolin-6-yl)-piperidin-1-yl-methanone

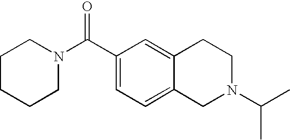

MS (ESI): mass calcd. for C₁₈H₂₆N₂O, 286.42; m/z found, 287.2 [M+H]⁺. ¹H NMR (CDCl₃): 7.14-7.11 (m, 2H), 7.05 (d, J=8.0, 1H), 3.73 (s, 2H), 3.69 (bs, 1.5H), 3.33 (bs, 1.5H), 2.95-2.90 (m, 3H), 2.78 (t, J=5.8, 2H), 1.94 (bs, 1H), 1.67 (bs, 4H), 1.49 (bs, 2H), 1.15 (d, J=7.0, 6H).

### Example 174

### (2-Isopropyl-1,2,3,4-tetrahydro-isoquinolin-6-yl)-morpholin-4-yl-methanone

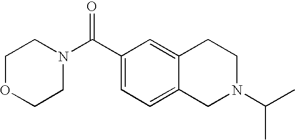

MS (ESI): mass calcd. for C₁₇H₂₄N₂O₂, 288.39; m/z found, 289.2 [M+H]⁺. ¹H NMR (CDCl₃): 7.16-7.12 (m, 2H), 7.07 (d, J=8.0, 1H), 3.74 (s, 2H), 3.70-3.44 (bm, 8H), 2.95-2.89 (m, 3H), 2.78 (t, J=6.0, 2H), 1.15 (d, J=6.5, 6H).

### Example 175

### (2-Isopropyl-1,2,3,4-tetrahydro-isoquinolin-6-yl)-(octahydro-pyrido[1,2-a]pyrazin-2-yl)-methanone

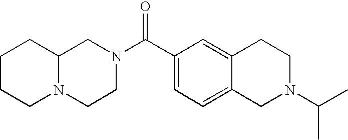

MS (ESI): mass calcd. for C₂₁H₃₁N₃O, 341.50; m/z found, 342.3 [M+H]⁺. ¹H NMR (CDCl₃): 7.15-7.12 (m, 2H), 7.06 (d, J=8.0, 1H), 4.65-4.49 (m, 1H), 3.74 (s, 2H), 3.70-3.50 (m, 1H), 3.27-2.96 (m, 1H), 2.95-2.77 (m, 7H), 2.64-2.55 (m, 1H), 2.24-2.10 (m, 1H), 2.08-1.60 (m, 6H), 1.32-1.18 (m, 2H), 1.15 (d, J=5.2, 6H).

### Example 176

### (4-tert-Butyl-piperidin-1-yl)-(2-isopropyl-1,2,3,4-tetrahydro-isoquinolin-6-yl)-methanone

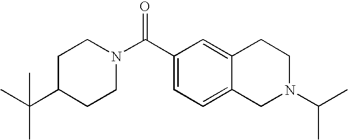

MS (ESI): mass calcd. for C₂₂H₃₄N₂O, 342.53; m/z found, 343.3 [M+H]⁺. ¹H NMR (CDCl₃): 7.14-7.11 (m, 2H), 7.05 (d, J=8.0, 1H), 4.79 (bs, 1H), 3.81 (bs, 1H), 3.74 (s, 2H), 2.95-2.87 (bm, 4H), 2.78 (t, J=5.8, 2H), 2.64 (bs, 1H), 1.93 (bs, 1H), 1.79 (bs, 1H), 1.60 (bs, 1H), 1.25-1.20 (m, 2H), 1.15 (d, J=6.5, 6H), 0.84 (s, 9H).

### Example 177

### (4-Cyclobutyl-[1,4]diazepan-1-yl)-(2-isopropyl-1,2,3,4-tetrahydro-isoquinolin-6-yl)-methanone

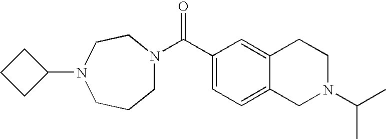

MS (ESI): mass calcd. for C₂₂H₃₃N₃O, 355.53; m/z found, 356.3 [M+H]⁺. ¹H NMR (CDCl₃): 7.11 (d, J=8.4, 2H), 7.04 (d, J=8.4, 1H), 4.76-3.72 (m, 4H), 3.47-3.41 (m, 2H), 2.95-2.76 (m, 4H), 2.77 (t, J=5.8, 2H), 2.62-2.60 (m, 1H), 2.51-2.48 (m, 1H), 2.43-2.36 (m, 2H), 2.08-1.58 (m, 8H), 1.14 (d, J=6.5, 6H).

### Example 178

### [4-(1-Hydroxy-1-methyl-ethyl)-piperidin-1-yl]-(2-isopropyl-1,2,3,4-tetrahydro-isoquinolin-6-yl)-methanone

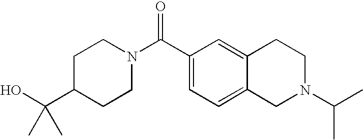

MS (ESI): mass calcd. for C₂₁H₃₂N₂O₂, 344.50; m/z found, 345.3 [M+H]⁺. ¹H NMR (CDCl₃): 7.12 (d, J=8.6, 2H), 7.05 (d, J=8.6, 1H), 4.80 (bs, 1H), 3.83 (bs, 1H), 3.73 (s, 2H), 2.96-2.87 (m, 4H), 2.78 (t, J=6.4, 2H), 2.61 (bs, 1H), 1.85-1.71 (m, 3H), 1.59-1.40 (m, 1H), 1.32-1.22 (m, 2H), 1.18 (s, 6H), 1.14 (d, J=6.5, 6H).

### Example 179

### Piperidin-1-yl-(2-propyl-1,2,3,4-tetrahydro-isoquinolin-6-yl)-methanone

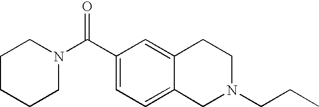

MS (ESI): mass calcd. for C₁₈H₂₆N₂O, 286.42; m/z found, 287.2 [M+H]⁺. ¹H NMR (CDCl₃): 7.13-7.11 (m, 2H), 7.03 (d, J=7.6, 1H), 3.68 (bs, 2H), 3.63 (s, 2H), 3.33 (bs, 2H), 2.92 (t, J=5.9, 2H), 2.71 (t, J=5.9, 2H), 2.50-2.46 (m, 2H), 1.67-1.57 (m, 6H), 1.50 (bs, 2H), 0.95 (t, J=7.4, 3H).

### Example 180

### Morpholin-4-yl-(2-propyl-1,2,3,4-tetrahydro-isoquinolin-6-yl)-methanone

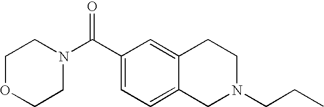

MS (ESI): mass calcd. for C₁₇H₂₄N₂O₂, 288.39; m/z found, 289.2 [M+H]⁺. ¹H NMR (CDCl₃): 7.16-7.12 (m, 2H), 7.05 (d, J=7.6, 1H), 3.68 (bs, 6H), 3.63 (s, 2H), 3.49-3.44 (m, 2H), 2.92 (t, J=5.9, 2H), 2.73 (t, J=5.9, 2H), 2.50-2.46 (m, 2H), 1.61 (p, J=7.5, 2H), 0.95 (t, J=7.5, 3H).

### Example 181

### (Octahydro-pyrido[1,2-a]pyrazin-2-yl)-(2-propyl-1,2,3,4-tetrahydro-isoquinolin-6-yl)-methanone

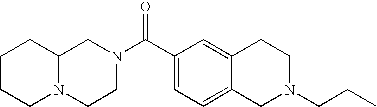

MS (ESI): mass calcd. for C₂₁H₃₁N₃O, 341.50; m/z found, 342.3 [M+H]⁺.

### Example 182

### (4-tert-Butyl-piperidin-1-yl)-(2-propyl-1,2,3,4-tetrahydro-isoquinolin-6-yl)-methanone

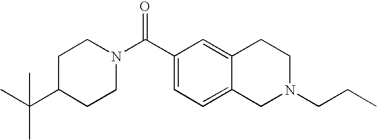

MS (ESI): mass calcd. for C₂₂H₃₄N₂O, 342.53; m/z found, 343.3 [M+H]⁺.

### Example 183

### (4-Cyclobutyl-[1,4]diazepan-1-yl)-(2-propyl-1,2,3,4-tetrahydro-isoquinolin-6-yl)-methanone

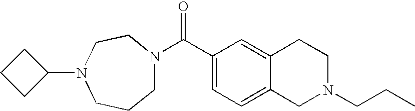

MS (ESI): mass calcd. for C₂₂H₃₃N₃O, 355.53; m/z found, 356.3 [M+H]⁺.

### Example 184

### [4-(1-Hydroxy-1-methyl-ethyl)-piperidin-1-yl]-(2-Propyl-1,2,3,4-tetrahydro-isoquinolin-6-yl)-methanone

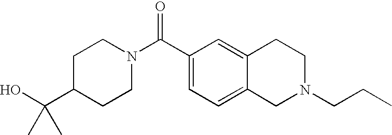

MS (ESI): mass calcd. for C₂₁H₃₂N₂O₂, 344.50; m/z found, 345.3 [M+H]⁺.

### Example 185

### (2-Cyclobutyl-1,2,3,4-tetrahydro-isoquinolin-6-yl)-piperidin-1-yl-methanone

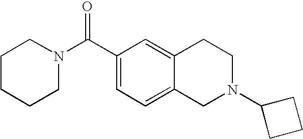

MS (ESI): mass calcd. for C₁₉H₂₆N₂O, 298.43; m/z found, 299.2 [M+H]⁺.

### Example 186

### (2-Cyclobutyl-1,2,3,4-tetrahydro-isoquinolin-6-yl)-morpholin-4-yl-methanone

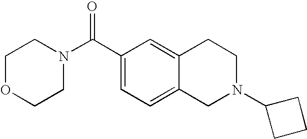

MS (ESI): mass calcd. for C₁₈H₂₄N₂O₂, 300.40; m/z found, 301.2 [M+H]⁺.

### Example 187

### (2-Cyclobutyl-1,2,3,4-tetrahydro-isoquinolin-6-yl)-(octahydro-pyrido[1,2-a]pyrazin-2-yl)-methanone

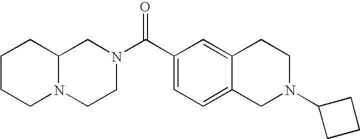

MS (ESI): mass calcd. for C₂₂H₃₁N₃O, 353.51; m/z found, 354.3 [M+H]⁺.

### Example 188

### (4-tert-Butyl-piperidin-1-yl)-(2-cyclobutyl-1,2,3,4-tetrahydro-isoquinolin-6-yl)-methanone

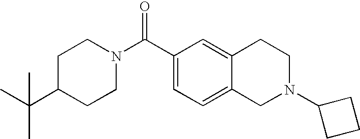

MS (ESI): mass calcd. for C₂₃H₃₄N₂O, 354.54; m/z found, 355.3 [M+H]⁺.

### Example 189

### (4-Cyclobutyl-[1,4]diazepan-1-yl)-(2-cyclobutyl-1,2,3,4-tetrahydro-isoquinolin-6-yl)-methanone

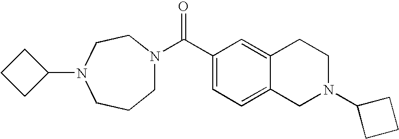

MS (ESI): mass calcd. for C₂₃H₃₃N₃O, 367.54; m/z found, 368.3 [M+H]⁺.

### Example 190

### (2-Cyclobutyl-1,2,3,4-tetrahydro-isoquinolin-6-yl)-[4-(1-hydroxy-1-methyl-ethyl)-piperidin-1-yl]-methanone

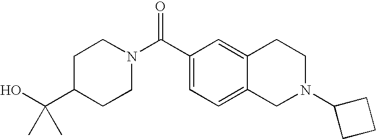

MS (ESI): mass calcd. for C₂₂H₃₂N₂O₂, 356.51; m/z found, 357.3 [M+H]⁺.

### Example 191

### (2-Cyclopentyl-1,2,3,4-tetrahydro-isoquinolin-6-yl)-piperidin-1-yl-methanone

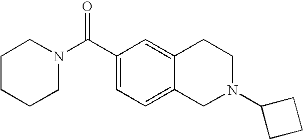

MS (ESI): mass calcd. for C₂₀H₂₈N₂O, 312.46; m/z found, 313.2 [M+H]⁺.

### Example 192

### (2-Cyclopentyl-1,2,3,4-tetrahydro-isoquinolin-6-yl)-(octahydro-pyrido[1,2-a]pyrazin-2-yl)-methanone

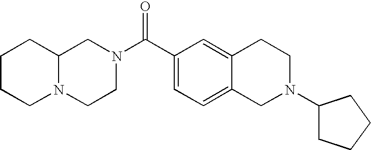

MS (ESI): mass calcd. for C₂₃H₃₃N₃O, 367.54; m/z found, 368.3 [M+H]⁺.

### Example 193

### (4-tert-Butyl-piperidin-1-yl)-(2-cyclopentyl-1,2,3,4-tetrahydro-isoquinolin-6-yl)-methanone

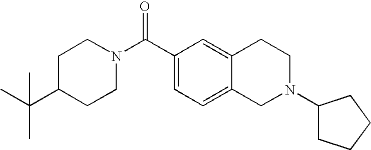

MS (ESI): mass calcd. for C₂₄H₃₆N₂O, 368.57; m/z found, 369.3 [M+H]⁺.

### Example 194

### (2-Cyclopentyl-1,2,3,4-tetrahydro-isoquinolin-6-yl)-[4-(1-hydroxy-1-methyl-ethyl)-piperidin-1-yl]-methanone

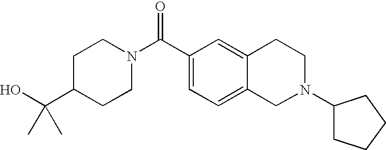

MS (ESI): mass calcd. for C₂₃H₃₄N₂O₂, 370.54; m/z found, 371.3 [M+H]⁺.

### Biological Methods:

### H₃ Receptor Binding (Human)

Binding of compounds to the cloned human H₃ receptors, stably expressed in SK-N-MC cells, was performed as described by Barbier, A. J. et al. (Br. J. Pharmacol. 2004, 143(5), 649-661). Data for compounds tested in this assay are presented in Table 1 as an average of the results obtained.

TABLE 1

| | Human H₃ |
|---|---|
| Ex. | Kᵢ (nM) |
| 1 | 8 |
| 2 | 230 |
| 3 | 165 |
| 4 | 28 |
| 5 | 3 |
| 6 | 3000 |
| 7 | 4000 |
| 8 | 11 |
| 9 | 14 |
| 10 | 16 |
| 11 | 1 |
| 12 | 7000 |
| 13 | >10000 |
| 14 | 20 |
| 15 | 4 |
| 16 | 18 |
| 17 | 15 |
| 18 | 5 |
| 19 | 29 |
| 20 | 19 |
| 21 | 28 |
| 22 | 5 |
| 23 | 114 |
| 24 | 75 |
| 25 | 15 |
| 26 | 2 |
| 27 | 9 |
| 28 | 16 |
| 29 | 80 |
| 30 | 36 |
| 31 | 87 |
| 32 | 5 |
| 33 | 8 |
| 34 | 14 |
| 35 | 1 |
| 36 | 1 |
| 37 | 1 |
| 38 | 1 |
| 39 | 1 |
| 40 | 1 |
| 41 | 15 |
| 42 | 10 |
| 43 | 20 |
| 44 | 19 |
| 45 | 6 |
| 46 | 4 |
| 47 | 16 |
| 48 | 18 |
| 49 | 46 |
| 50 | 12 |
| 51 | 14 |
| 52 | 15 |
| 53 | 9 |
| 54 | 9 |
| 55 | 22 |
| 56 | 23 |
| 57 | 14 |
| 58 | 17 |
| 59 | 12 |
| 60 | 21 |
| 61 | 7 |
| 62 | 24 |
| 63 | 61 |
| 64 | 100 |
| 65 | 100 |
| 66 | 43 |
| 67 | 53 |
| 68 | 32 |
| 69 | 91 |
| 70 | 65 |
| 71 | 48 |
| 72 | 80 |
| 73 | 109 |
| 74 | 109 |
| 75 | 77 |
| 76 | 100 |
| 77 | 180 |
| 78 | 70 |
| 79 | 169 |
| 80 | 70 |
| 81 | 65 |
| 82 | 55 |
| 83 | 257 |
| 84 | 180 |
| 85 | 118 |
| 86 | 27 |
| 87 | 23 |
| 88 | 24 |
| 89 | 21 |
| 90 | 19 |
| 91 | 25 |
| 92 | 23 |
| 93 | 17 |
| 94 | 22 |
| 95 | 16 |
| 96 | 15 |
| 97 | 38 |
| 98 | 38 |
| 99 | 20 |
| 100 | 35 |
| 101 | 34 |
| 102 | 25 |
| 103 | 22 |
| 104 | 16 |
| 105 | 16 |
| 106 | 17 |
| 107 | 34 |
| 108 | 28 |
| 109 | 37 |
| 110 | 47 |
| 111 | 24 |
| 112 | 20 |
| 113 | 22 |
| 114 | 13 |
| 115 | 11 |
| 116 | 18 |
| 117 | 13 |
| 118 | 8 |
| 119 | 11 |
| 120 | 12 |
| 121 | 5 |
| 122 | 7 |
| 123 | 16 |
| 124 | 24 |
| 125 | 17 |
| 126 | 9 |
| 127 | 27 |
| 128 | 19 |
| 129 | 18 |
| 130 | 10 |
| 131 | 78 |
| 132 | 100 |
| 133 | 2 |
| 134 | 1 |
| 135 | 1 |
| 136 | 1 |
| 137 | 1 |
| 138 | 1 |
| 139 | 1 |
| 140 | 2 |
| 141 | 1 |
| 142 | 1 |
| 143 | 1 |
| 144 | 1 |
| 145 | 1 |
| 146 | 1 |
| 147 | 1 |
| 148 | 1 |
| 149 | 109 |
| 150 | 9 |
| 151 | 5200 |
| 152 | 9 |
| 153 | 54 |
| 154 | 441 |
| 155 | 650 |
| 156 | 30 |
| 157 | 110 |
| 158 | 4088 |
| 159 | 37 |
| 160 | 84 |
| 161 | 421 |
| 162 | 4 |
| 163 | 170 |
| 164 | 1150 |
| 165 | 300 |
| 166 | 45 |
| 167 | 1393 |
| 168 | 13 |
| 169 | 269 |
| 170 | 860 |
| 171 | 23 |
| 172 | 2800 |
| 173 | 1800 |
| 174 | 2135 |
| 175 | 2 |
| 176 | 250 |
| 177 | 1 |
| 178 | 3300 |
| 179 | 10000 |
| 180 | 9000 |
| 181 | 2 |
| 182 | 340 |
| 183 | 1 |
| 184 | 4200 |
| 185 | 2100 |
| 186 | 1800 |
| 187 | 3 |
| 188 | 340 |
| 189 | 1 |
| 190 | 10000 |
| 191 | 2200 |
| 192 | 1 |
| 193 | 240 |
| 194 | 2700 |

### H₃ Receptor Binding (Rat)

A rat brain without cerebellum (Zivic Laboratories Inc., Pittsburgh, Pa.) was homogenized in 50 mM Tris-HCl/5 mM EDTA and centrifuged at 1,000 rpm for 5 min. The supernatant was removed and recentrifuged at 15,000 rpm for 30 min. Pellets were rehomogenized in 50 mM Tris/5 mM EDTA (pH 7.4). Membranes were incubated with 0.8 nM N-[³H]-α-methylhistamine plus/minus test compounds for 60 min at 25° C. and harvested by rapid filtration over GF/C glass fiber filters (pretreated with 0.3% polyethylenimine) followed by four washes with buffer. Nonspecific binding was defined in the presence of 100 μM histamine. Inhibitory concentration (responsible for 50% inhibition of maximal effect, IC₅₀) values were determined by a single site curve-fitting program (GraphPad, San Diego, Calif.) and converted to Kᵢ values based on a N-[³H]-α-methylhistamine dissociation constant (K_{d}) of 0.8 nM. Data for compounds tested in this assay are presented in Table 2 as an average of the results obtained.

TABLE 2

| | Rat H₃ |
|---|---|
| Ex. | Kᵢ (nM) |
| 18 | 2 |
| 27 | 84 |
| 32 | 135 |
| 33 | 123 |
| 133 | 18 |
| 135 | 11 |
| 136 | 16 |
| 137 | 8 |
| 139 | 7 |
| 142 | 28 |
| 144 | 32 |
| 177 | 1 |
| 187 | 30 |

### Cyclic AMP Accumulation

Sublines of SK-N-MC cells were created that expressed a reporter construct and either the human or rat H₃ receptor. The pA₂ values were obtained as described by Barbier et al. (2004). Data for compounds tested in these assays are presented in Table 3, as an average of the results obtained (NT=not tested).

TABLE 3

| Ex. | Human pA₂ | Rat pA₂ |
|---|---|---|
| 5 | 8.81 | 7.80 |
| 11 | 9.18 | 8.35 |
| 15 | 8.98 | NT |
| 18 | 8.89 | 8.62 |
| 22 | 7.98 | 7.51 |
| 26 | 7.75 | NT |
| 32 | 8.07 | 7.82 |
| 33 | NT | 7.86 |
| 35 | 9.29 | 8.63 |
| 36 | 9.31 | 8.42 |
| 39 | 9.42 | 8.52 |
| 40 | 9.45 | 8.54 |
| 45 | 8.55 | 7.69 |
| 46 | 8.55 | 7.74 |

What is claimed is:

1. A compound of Formula (I): 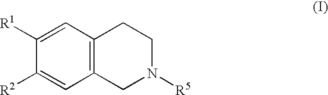
wherein
one of R¹ and R² is -L-N(R³)R⁴ and the other is —H;
where L is C(O) or CH₂; and
—N(R³)R⁴ is one of the following moieties: 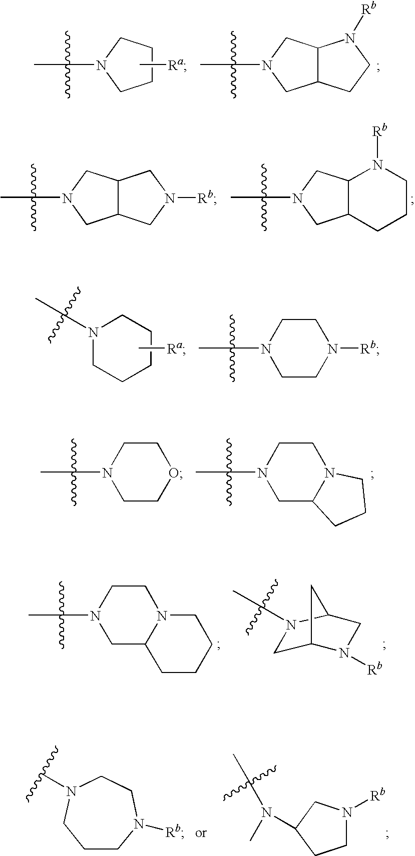
where R^{a} is —H, —C₁₋₄alkyl, —C₁₋₄alkyl-OH, —OH, —NR^{c}R^{d}, or —CH₂NR^{c}R^{d};
R^{c} and R^{d} are each independently H or —C₁₋₄alkyl, or R^{c} and R^{d} taken together with the nitrogen to which they are attached form pyrrolidinyl, piperidinyl, or morpholinyl; and
R^{b} is —C₁₋₄alkyl or —C₃₋₇cycloalkyl;
R⁵ is —H, C₁₋₄alkyl, C₃₋₇cycloalkyl, —CH₂-phenyl, —CH₂-(monocyclic heteroaryl), —C(O)—C₁₋₄alkyl, —C(O)—C₃₋₇cycloalkyl, —C(O)-(monocyclic heterocycloalkyl), —C(O)-phenyl, —C(O)-(monocyclic heteroaryl), —C(O)CH₂—C₃₋₇cycloalkyl, —C(O)CH₂-phenyl, —C(O)CH₂-(monocyclic heteroaryl), —CO₂C₁₋₄alkyl, —SO₂C₁₋₄alkyl, or —SO₂-phenyl;
where each cycloalkyl, phenyl, monocyclic heteroaryl, or monocyclic heterocycloalkyl group in R⁵ is unsubstituted or substituted with one or two substituents independently selected from the group consisting of —C₁₋₄alkyl, —CF₃, halo, —CN, —NO₂, —OH, —OC₁₋₄alkyl, —C₃₋₇cycloalkyl, and —NR^{x}R^{y};
R^{x} and R^{y} are each independently H or —C₁₋₄alkyl;
with the proviso that the compound of Formula (I) comprises at least one nitrogen atom which is not part of an amide, carbamoyl, cyano, nitro, or sulfonamide group;
or a pharmaceutically acceptable salt, a pharmaceutically acceptable prodrug, or a pharmaceutically active metabolite thereof.

2. A compound as defined in claim 1, wherein R¹ is -L-N(R³)R⁴ and R² is —H.

3. A compound as defined in claim 1, wherein L is C(O).

4. A compound as defined in claim 1, wherein —N(R³)R⁴ is one of the following moieties: 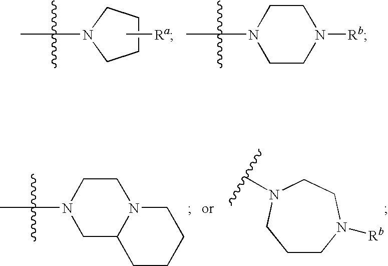
where R^{a} and R^{b} are as defined for Formula (I).

5. A compound as defined in claim 1, wherein R^{a} is —H, methyl, ethyl, isopropyl, tert-butyl, 1-hydroxy-1-methyl-ethyl, —OH, dimethylamino, piperidin-1-yl, morpholin-1-yl, or 2-pyrrolidin-1-ylmethyl.

6. A compound as defined in claim 1, wherein R^{b} is methyl, ethyl, isopropyl, tert-butyl, cyclopropyl, cyclobutyl, cyclopentyl, or cyclohexyl.

7. A compound as defined in claim 1, wherein —N(R³)R⁴ is 4-isopropyl-[1,4]diazepan-1-yl, piperidin-1-yl, morpholin-1-yl, 4-cyclopentyl-piperazin-1-yl, 4-cyclohexyl-piperazin-1-yl, octahydro-pyrido[1,2-a]pyrazin-2-yl, 4-cyclobutyl-piperazin-1-yl, 4-isopropyl-piperazin-1-yl, 4-cyclopropyl-piperazin-1-yl, 4-cyclobutyl-[1,4]diazepan-1-yl, 2-pyrrolidin-1-ylmethyl-pyrrolidin-1-yl, 4-(tetrahydro-furan-2-ylmethyl)-piperazin-1-yl, hexahydro-pyrrolo[1,2-a]pyrazin-2-yl, 4-dimethylamino-piperidin-1-yl, 3-dimethylamino-pyrrolidin-1-yl, [1,4′]bipiperidin-1′-yl, 4-morpholin-4-yl-piperidin-1-yl, N-methyl-N-(1-methyl-pyrrolidin-3-yl), 2-tert-butoxy-carbonyl-2,5-diaza-bicyclo[2.2.1]hept-5-yl, 1-tert-butoxy-carbonyl-hexahydro-pyrrolo[3,4-b]pyrrol-5-yl, 2-tert-butoxy-carbonyl-hexahydro-pyrrolo[3,4-c]pyrrol-5-yl, hexahydro-pyrrolo[3,4-c]pyrrol-2-yl, 2,5-diaza-bicyclo[2.2.1]hept-2-yl, hexahydro-pyrrolo[3,4-b]pyrrol-5-yl, 5-cyclobutyl-hexahydro-pyrrolo[3,4-c]pyrrol-2-yl, 5-cyclobutyl-2,5-diaza-bicyclo[2.2.1]hept-2-yl, 1-cyclobutyl-hexahydro-pyrrolo[3,4-b]pyrrol-5-yl, 4-tert-butyl-piperidin-1-yl, or 4-(1-hydroxy-1-methyl-ethyl)-piperidin-1-yl.

8. A compound as defined in claim 1, wherein —N(R³)R⁴ is 4-isopropyl-[1,4]diazepan-1-yl, octahydro-pyrido[1,2-a]pyrazin-2-yl, 4-cyclobutyl-piperazin-1-yl, 4-isopropyl-piperazin-1-yl, 4-cyclopropyl-piperazin-1-yl, 4-cyclobutyl-[1,4]diazepan-1-yl, or 2-pyrrolidin-1-ylmethyl-pyrrolidin-1-yl.

9. A compound as defined in claim 1, wherein R⁵ is —H, methyl, ethyl, propyl, or isopropyl.

10. A compound as defined in claim 1, wherein R⁵ is cyclopropyl, cyclobutyl, or cyclopentyl.

11. A compound as defined in claim 1, wherein R⁵ is benzyl, thiophen-3-ylmethyl, or furan-3-ylmethyl.

12. A compound as defined in claim 1, wherein R⁵ is acetyl, propionyl, butyryl, or 2,2-dimethylpropionyl.

13. A compound as defined in claim 1, wherein R⁵ is cyclopropanecarbonyl, cyclobutanecarbonyl, cyclopentanecarbonyl, or cyclohexanecarbonyl.

14. A compound as defined in claim 1, wherein R⁵ is tetrahydrofuran-2-carbonyl, tetrahydrofuran-3-carbonyl, or piperidine-4-carbonyl.

15. A compound as defined in claim 1, wherein R⁵ is benzoyl, furan-3-carbonyl, or thiophen-3-carbonyl.

16. A compound as defined in claim 1, wherein R⁵ is 2-cyclopentyl-acetyl, phenylacetyl, or 2-furan-2-yl-acetyl.

17. A compound as defined in claim 1, wherein R⁵ is tert-butoxycarbonyl.

18. A compound as defined in claim 1, wherein R⁵ is ethanesulfonyl, propane-1-sulfonyl, propane-2-sulfonyl, or benzenesulfonyl.

19. A compound selected from the group consisting of:
6-(4-Isopropyl-[1,4]diazepane-1-carbonyl)-3,4-dihydro-1H-isoquinoline-2-carboxylic acid tert-butyl ester;
6-(4-Cyclopentyl-piperazine-1-carbonyl)-3,4-dihydro-1H-isoquinoline-2-carboxylic acid tert-butyl ester;
6-(4-Cyclohexyl-piperazine-1-carbonyl)-3,4-dihydro-1H-isoquinoline-2-carboxylic acid tert-butyl ester;
6-(Octahydro-pyrido[1,2-a]pyrazine-2-carbonyl)-3,4-dihydro-1H-isoquinoline-2-carboxylic acid tert-butyl ester;
(4-Isopropyl-[1,4]diazepan-1-yl)-(1,2,3,4-tetrahydro-isoquinolin-6-yl)-methanone;
Piperidin-1-yl-(1,2,3,4-tetrahydro-isoquinolin-6-yl)-methanone;
Morpholin-4-yl-(1,2,3,4-tetrahydro-isoquinolin-6-yl)-methanone;
(4-Cyclopentyl-piperazin-1-yl)-(1,2,3,4-tetrahydro-isoquinolin-6-yl)-methanone;
(4-Cyclohexyl-piperazin-1-yl)-(1,2,3,4-tetrahydro-isoquinolin-6-yl)-methanone;
(Octahydro-pyrido[1,2-a]pyrazin-2-yl)-(1,2,3,4-tetrahydro-isoquinolin-6-yl)-methanone;
(2-Benzyl-1,2,3,4-tetrahydro-isoquinolin-6-yl)-(4-isopropyl-[1,4]diazepan-1-yl)-methanone;
(2-Benzyl-1,2,3,4-tetrahydro-isoquinolin-6-yl)-piperidin-1-yl-methanone;
(2-Benzyl-1,2,3,4-tetrahydro-isoquinolin-6-yl)-morpholin-4-yl-methanone;
(4-Cyclobutyl-piperazin-1-yl)-[2-(4-trifluoromethyl-benzyl)-1,2,3,4-tetrahydro-isoquinolin-6-yl]-methanone;
(4-Cyclobutyl-piperazin-1-yl)-(2-thiophen-3-ylmethyl-1,2,3,4-tetrahydro-isoquinolin-6-yl)-methanone;
(4-Cyclobutyl-piperazin-1-yl)-[2-(3,4-dichloro-benzyl)-1,2,3,4-tetrahydro-isoquinolin-6-yl]-methanone;
[2-(4-Chloro-benzyl)-1,2,3,4-tetrahydro-isoquinolin-6-yl]-(4-cyclobutyl-piperazin-1-yl)-methanone;
(2-Benzyl-1,2,3,4-tetrahydro-isoquinolin-6-yl)-(4-cyclobutyl-piperazin-1-yl)-methanone;
[2-(3,4-Dichloro-benzyl)-1,2,3,4-tetrahydro-isoquinolin-6-yl]-(4-isopropyl-piperazin-1-yl)-methanone;
(4-Isopropyl-piperazin-1-yl)-[2-(4-trifluoromethyl-benzyl)-1,2,3,4-tetrahydro-isoquinolin-6-yl]-methanone;
(2-Benzyl-1,2,3,4-tetrahydro-isoquinolin-6-yl)-(4-isopropyl-piperazin-1-yl)-methanone;
[2-(4-Chloro-benzyl)-1,2,3,4-tetrahydro-isoquinolin-6-yl]-(4-isopropyl-piperazin-1-yl)-methanone;
(4-Cyclopropyl-piperazin-1-yl)-[2-(4-trifluoromethyl-benzyl)-1,2,3,4-tetrahydro-isoquinolin-6-yl]-methanone;
[2-(4-Chloro-benzyl)-1,2,3,4-tetrahydro-isoquinolin-6-yl]-(4-cyclopropyl-piperazin-1-yl)-methanone;
4-[6-(4-Cyclobutyl-piperazine-1-carbonyl)-3,4-dihydro-1H-isoquinolin-2-ylmethyl]-benzonitrile;
(2-Benzyl-1,2,3,4-tetrahydro-isoquinolin-7-yl)-(4-cyclobutyl-piperazin-1-yl)-methanone;
(2-Benzoyl-1,2,3,4-tetrahydro-isoquinolin-6-yl)-(4-cyclobutyl-piperazin-1-yl)-methanone;
(2-Benzoyl-1,2,3,4-tetrahydro-isoquinolin-6-yl)-(4-isopropyl-piperazin-1-yl)-methanone;
1-[6-(4-Isopropyl-piperazine-1-carbonyl)-3,4-dihydro-1H-isoquinolin-2-yl]-ethanone;
1-[6-(4-Cyclobutyl-piperazine-1-carbonyl)-3,4-dihydro-1H-isoquinolin-2-yl]-ethanone;
Cyclobutyl-[6-(4-cyclobutyl-piperazine-1-carbonyl)-3,4-dihydro-1H-isoquinolin-2-yl]-methanone;
[6-(4-Cyclobutyl-piperazine-1-carbonyl)-3,4-dihydro-1H-isoquinolin-2-yl]-cyclopentyl-methanone;
[6-(4-Cyclobutyl-piperazine-1-carbonyl)-3,4-dihydro-1H-isoquinolin-2-yl]-cyclohexyl-methanone;
[6-(4-Cyclobutyl-piperazine-1-carbonyl)-3,4-dihydro-1H-isoquinolin-2-yl]-cyclopropyl-methanone;
[6-(4-Cyclobutyl-[1,4]diazepane-1-carbonyl)-3,4-dihydro-1H-isoquinolin-2-yl]-phenyl-methanone;
[7-(4-Cyclobutyl-[1,4]diazepane-1-carbonyl)-3,4-dihydro-1H-isoquinolin-2-yl]-phenyl-methanone;
[7-(4-Cyclobutyl-[1,4]diazepane-1-carbonyl)-3,4-dihydro-1H-isoquinolin-2-yl]-cyclopentyl-methanone;
[7-(4-Cyclobutyl-[1,4]diazepane-1-carbonyl)-3,4-dihydro-1H-isoquinolin-2-yl]-cyclohexyl-methanone;
[6-(4-Cyclobutyl-[1,4]diazepane-1-carbonyl)-3,4-dihydro-1H-isoquinolin-2-yl]-cyclopentyl-methanone;
[6-(4-Cyclobutyl-[1,4]diazepane-1-carbonyl)-3,4-dihydro-1H-isoquinolin-2-yl]-cyclohexyl-methanone;
1-[6-(4-Cyclobutyl-piperazine-1-carbonyl)-3,4-dihydro-1H-isoquinolin-2-yl]-2,2-dimethyl-propan-1-one;
(2-Chloro-phenyl)-[6-(4-cyclobutyl-piperazine-1-carbonyl)-3,4-dihydro-1H-isoquinolin-2-yl]-methanone;
1-[6-(4-Cyclobutyl-piperazine-1-carbonyl)-3,4-dihydro-1H-isoquinolin-2-yl]-2-cyclopentyl-ethanone;
[6-(4-Cyclobutyl-piperazine-1-carbonyl)-3,4-dihydro-1H-isoquinolin-2-yl]-furan-3-yl-methanone;
(S)-1-[6-(2-Pyrrolidin-1-ylmethyl-pyrrolidine-1-carbonyl)-3,4-dihydro-1H-isoquinolin-2-yl]-propan-1-one;
(S)-1-[6-(2-Pyrrolidin-1-ylmethyl-pyrrolidine-1-carbonyl)-3,4-dihydro-1H-isoquinolin-2-yl]-butan-1-one;
(S)-2,2-Dimethyl-1-[6-(2-pyrrolidin-1-ylmethyl-pyrrolidine-1-carbonyl)-3,4-dihydro-1H-isoquinolin-2-yl]-propan-1-one;
(S)-Phenyl-[6-(2-pyrrolidin-1-ylmethyl-pyrrolidine-1-carbonyl)-3,4-dihydro-1H-isoquinolin-2-yl]-methanone;
(S)-(4-tert-Butyl-phenyl)-[6-(2-pyrrolidin-1-ylmethyl-pyrrolidine-1-carbonyl)-3,4-dihydro-1H-isoquinolin-2-yl]-methanone;
(S)-(2-Chloro-phenyl)-[6-(2-pyrrolidin-1-ylmethyl-pyrrolidine-1-carbonyl)-3,4-dihydro-1H-isoquinolin-2-yl]-methanone;
(S)-(3-Chloro-phenyl)-[6-(2-pyrrolidin-1-ylmethyl-pyrrolidine-1-carbonyl)-3,4-dihydro-1H-isoquinolin-2-yl]-methanone;
(S)-3-[6-(2-Pyrrolidin-1-ylmethyl-pyrrolidine-1-carbonyl)-3,4-dihydro-1H-isoquinoline-2-carbonyl]-benzonitrile;
(S)-4-[6-(2-Pyrrolidin-1-ylmethyl-pyrrolidine-1-carbonyl)-3,4-dihydro-1H-isoquinoline-2-carbonyl]-benzonitrile;
(S)-[6-(2-Pyrrolidin-1-ylmethyl-pyrrolidine-1-carbonyl)-3,4-dihydro-1H-isoquinolin-2-yl]-o-tolyl-methanone;
(S)-[6-(2-Pyrrolidin-1-ylmethyl-pyrrolidine-1-carbonyl)-3,4-dihydro-1H-isoquinolin-2-yl]-p-tolyl-methanone;
(S)-(2-Fluoro-phenyl)-[6-(2-pyrrolidin-1-ylmethyl-pyrrolidine-1-carbonyl)-3,4-dihydro-1H-isoquinolin-2-yl]-methanone;
(S)-(3-Fluoro-phenyl)-[6-(2-pyrrolidin-1-ylmethyl-pyrrolidine-1-carbonyl)-3,4-dihydro-1H-isoquinolin-2-yl]-methanone;
(S)-(4-Fluoro-phenyl)-[6-(2-pyrrolidin-1-ylmethyl-pyrrolidine-1-carbonyl)-3,4-dihydro-1H-isoquinolin-2-yl]-methanone;
(S)-(3-Methoxy-phenyl)-[6-(2-pyrrolidin-1-ylmethyl-pyrrolidine-1-carbonyl)-3,4-dihydro-1H-isoquinolin-2-yl]-methanone;
(S)-(4-Methoxy-phenyl)-[6-(2-pyrrolidin-1-ylmethyl-pyrrolidine-1-carbonyl)-3,4-dihydro-1H-isoquinolin-2-yl]-methanone;
(S)-2-Phenyl-1-[6-(2-pyrrolidin-1-ylmethyl-pyrrolidine-1-carbonyl)-3,4-dihydro-1H-isoquinolin-2-yl]-ethanone;
(4-Cyclobutyl-piperazin-1-yl)-[2-(3-fluoro-benzoyl)-1,2,3,4-tetrahydro-isoquinolin-6-yl]-methanone;
1-[6-(4-Cyclopentyl-piperazine-1-carbonyl)-3,4-dihydro-1H-isoquinolin-2-yl]-propan-1-one;
1-[6-(4-Cyclopentyl-piperazine-1-carbonyl)-3,4-dihydro-1H-isoquinolin-2-yl]-2,2-dimethyl-propan-1-one;
(2-Benzoyl-1,2,3,4-tetrahydro-isoquinolin-6-yl)-(4-cyclopentyl-piperazin-1-yl)-methanone;
(4-Cyclopentyl-piperazin-1-yl)-[2-(2-fluoro-benzoyl)-1,2,3,4-tetrahydro-isoquinolin-6-yl]-methanone;
(4-Cyclopentyl-piperazin-1-yl)-[2-(4-fluoro-benzoyl)-1,2,3,4-tetrahydro-isoquinolin-6-yl]-methanone;
[2-(2-Chloro-benzoyl)-1,2,3,4-tetrahydro-isoquinolin-6-yl]-(4-cyclopentyl-piperazin-1-yl)-methanone;
[2-(4-Chloro-benzoyl)-1,2,3,4-tetrahydro-isoquinolin-6-yl]-(4-cyclopentyl-piperazin-1-yl)-methanone;
(4-Cyclopentyl-piperazin-1-yl)-[2-(4-methyl-benzoyl)-1,2,3,4-tetrahydro-isoquinolin-6-yl]-methanone;
1-[6-(4-Cyclopentyl-piperazine-1-carbonyl)-3,4-dihydro-1H-isoquinolin-2-yl]-2-(4-fluoro-phenyl)-ethanone;
(4-Cyclohexyl-piperazin-1-yl)-[2-(2-fluoro-benzoyl)-1,2,3,4-tetrahydro-isoquinolin-6-yl]-methanone;
(4-Cyclohexyl-piperazin-1-yl)-[2-(3-fluoro-benzoyl)-1,2,3,4-tetrahydro-isoquinolin-6-yl]-methanone;
(4-Cyclohexyl-piperazin-1-yl)-[2-(4-fluoro-benzoyl)-1,2,3,4-tetrahydro-isoquinolin-6-yl]-methanone;
[2-(2-Chloro-benzoyl)-1,2,3,4-tetrahydro-isoquinolin-6-yl]-(4-cyclohexyl-piperazin-1-yl)-methanone;
[2-(3-Chloro-benzoyl)-1,2,3,4-tetrahydro-isoquinolin-6-yl]-(4-cyclohexyl-piperazin-1-yl)-methanone;
[2-(4-Chloro-benzoyl)-1,2,3,4-tetrahydro-isoquinolin-6-yl]-(4-cyclohexyl-piperazin-1-yl)-methanone;
(4-Cyclohexyl-piperazin-1-yl)-[2-(2-methoxy-benzoyl)-1,2,3,4-tetrahydro-isoquinolin-6-yl]-methanone;
(4-Cyclohexyl-piperazin-1-yl)-[2-(3-methoxy-benzoyl)-1,2,3,4-tetrahydro-isoquinolin-6-yl]-methanone;
(3-[6-(4-Cyclohexyl-piperazine-1-carbonyl)-3,4-dihydro-1H-isoquinoline-2-carbonyl]-benzonitrile;
4-[6-(4-Cyclohexyl-piperazine-1-carbonyl)-3,4-dihydro-1H-isoquinoline-2-carbonyl]-benzonitrile;
(4-Cyclohexyl-piperazin-1-yl)-[2-(2-methyl-benzoyl)-1,2,3,4-tetrahydro-isoquinolin-6-yl]-methanone;
(4-Cyclohexyl-piperazin-1-yl)-[2-(4-methyl-benzoyl)-1,2,3,4-tetrahydro-isoquinolin-6-yl]-methanone;
[2-(4-tert-Butyl-benzoyl)-1,2,3,4-tetrahydro-isoquinolin-6-yl]-(4-cyclohexyl-piperazin-1-yl)-methanone;
(2-Benzoyl-1,2,3,4-tetrahydro-isoquinolin-6-yl)-(4-cyclohexyl-piperazin-1-yl)-methanone;
[2-(2-Fluoro-benzoyl)-1,2,3,4-tetrahydro-isoquinolin-6-yl]-(octahydro-pyrido[1,2-a]pyrazin-2-yl)-methanone;
[2-(3-Fluoro-benzoyl)-1,2,3,4-tetrahydro-isoquinolin-6-yl]-(octahydro-pyrido[1,2-a]pyrazin-2-yl)-methanone;
[2-(4-Fluoro-benzoyl)-1,2,3,4-tetrahydro-isoquinolin-6-yl]-(octahydro-pyrido[1,2-a]pyrazin-2-yl)-methanone;
[2-(2-Chloro-benzoyl)-1,2,3,4-tetrahydro-isoquinolin-6-yl]-(octahydro-pyrido[1,2-a]pyrazin-2-yl)-methanone;
[2-(3-Chloro-benzoyl)-1,2,3,4-tetrahydro-isoquinolin-6-yl]-(octahydro-pyrido[1,2-a]pyrazin-2-yl)-methanone;
[2-(4-Chloro-benzoyl)-1,2,3,4-tetrahydro-isoquinolin-6-yl]-(octahydro-pyrido[1,2-a]pyrazin-2-yl)-methanone;
[2-(2-Methoxy-benzoyl)-1,2,3,4-tetrahydro-isoquinolin-6-yl]-(octahydro-pyrido[1,2-a]pyrazin-2-yl)-methanone;
[2-(3-Methoxy-benzoyl)-1,2,3,4-tetrahydro-isoquinolin-6-yl]-(octahydro-pyrido[1,2-a]pyrazin-2-yl)-methanone;
3-[6-(Octahydro-pyrido[1,2-a]pyrazine-2-carbonyl)-3,4-dihydro-1H-isoquinoline-2-carbonyl]-benzonitrile;
4-[6-(Octahydro-pyrido[1,2-a]pyrazine-2-carbonyl)-3,4-dihydro-1H-isoquinoline-2-carbonyl]-benzonitrile;
[2-(2-Methyl-benzoyl)-1,2,3,4-tetrahydro-isoquinolin-6-yl]-(octahydro-pyrido[1,2-a]pyrazin-2-yl)-methanone;
[2-(4-Methyl-benzoyl)-1,2,3,4-tetrahydro-isoquinolin-6-yl]-(octahydro-pyrido[1,2-a]pyrazin-2-yl)-methanone;
[2-(4-tert-Butyl-benzoyl)-1,2,3,4-tetrahydro-isoquinolin-6-yl]-(octahydro-pyrido[1,2-a]pyrazin-2-yl)-methanone;
(2-Benzoyl-1,2,3,4-tetrahydro-isoquinolin-6-yl)-(octahydro-pyrido[1,2-a]pyrazin-2-yl)-methanone;
(4-Cyclobutyl-piperazin-1-yl)-(2-ethanesulfonyl-1,2,3,4-tetrahydro-isoquinolin-6-yl)-methanone;
(4-Cyclobutyl-piperazin-1-yl)-[2-(propane-1-sulfonyl)-1,2,3,4-tetrahydro-isoquinolin-6-yl]-methanone;
(4-Cyclobutyl-piperazin-1-yl)-[2-(propane-2-sulfonyl)-1,2,3,4-tetrahydro-isoquinolin-6-yl]-methanone;
(2-Benzenesulfonyl-1,2,3,4-tetrahydro-isoquinolin-6-yl)-(4-cyclobutyl-piperazin-1-yl)-methanone;
(4-Cyclobutyl-piperazin-1-yl)-[2-(4-fluoro-benzenesulfonyl)-1,2,3,4-tetrahydro-isoquinolin-6-yl]-methanone;
[2-(4-Chloro-benzoyl)-1,2,3,4-tetrahydro-isoquinolin-6-yl]-(4-cyclobutyl-piperazin-1-yl)-methanone;
(4-Isopropyl-piperazin-1-yl)-[2-(thiophene-3-carbonyl)-1,2,3,4-tetrahydro-isoquinolin-6-yl]-methanone;
[2-(4-Hydroxy-benzoyl)-1,2,3,4-tetrahydro-isoquinolin-6-yl]-(4-isopropyl-piperazin-1-yl)-methanone;
(4-Isopropyl-piperazin-1-yl)-[2-(4-methoxy-benzoyl)-1,2,3,4-tetrahydro-isoquinolin-6-yl]-methanone;
(4-Isopropyl-piperazin-1-yl)-[2-(4-methyl-benzoyl)-1,2,3,4-tetrahydro-isoquinolin-6-yl]-methanone;
[2-(4-Chloro-benzoyl)-1,2,3,4-tetrahydro-isoquinolin-6-yl]-(4-isopropyl-piperazin-1-yl)-methanone;
[2-(3,4-Dichloro-benzoyl)-1,2,3,4-tetrahydro-isoquinolin-6-yl]-(4-isopropyl-piperazin-1-yl)-methanone;
(4-Cyclobutyl-piperazin-1-yl)-[2-(4-methoxy-benzoyl)-1,2,3,4-tetrahydro-isoquinolin-6-yl]-methanone;
(4-Cyclobutyl-piperazin-1-yl)-[2-(4-methyl-benzoyl)-1,2,3,4-tetrahydro-isoquinolin-6-yl]-methanone;
(4-Cyclobutyl-piperazin-1-yl)-[2-(3,4-dichloro-benzoyl)-1,2,3,4-tetrahydro-isoquinolin-6-yl]-methanone;
(4-Cyclobutyl-piperazin-1-yl)-[2-(thiophene-3-carbonyl)-1,2,3,4-tetrahydro-isoquinolin-6-yl]-methanone;
[6-(4-Cyclobutyl-piperazine-1-carbonyl)-3,4-dihydro-1H-isoquinolin-2-yl]-(3-dimethylamino-phenyl)-methanone;
[6-(4-Cyclobutyl-piperazine-1-carbonyl)-3,4-dihydro-1H-isoquinolin-2-yl]-(4-dimethylamino-phenyl)-methanone;
[6-(4-Cyclobutyl-piperazine-1-carbonyl)-3,4-dihydro-1H-isoquinolin-2-yl]-(2,4-dichloro-phenyl)-methanone;
(3-Chloro-phenyl)-[6-(4-cyclobutyl-piperazine-1-carbonyl)-3,4-dihydro-1H-isoquinolin-2-yl]-methanone;
[6-(4-Cyclobutyl-piperazine-1-carbonyl)-3,4-dihydro-1H-isoquinolin-2-yl]-m-tolyl-methanone;
[6-(4-Cyclobutyl-piperazine-1-carbonyl)-3,4-dihydro-1H-isoquinolin-2-yl]-(3-nitro-phenyl)-methanone;
[6-(4-Cyclobutyl-piperazine-1-carbonyl)-3,4-dihydro-1H-isoquinolin-2-yl]-(4-nitro-phenyl)-methanone;
[6-(4-Cyclobutyl-piperazine-1-carbonyl)-3,4-dihydro-1H-isoquinolin-2-yl]-(4-hydroxy-phenyl)-methanone;
(4-Cyclobutyl-piperazin-1-yl)-[2-(4-fluoro-3-hydroxy-benzoyl)-1,2,3,4-tetrahydro-isoquinolin-6-yl]-methanone;
(4-Cyclobutyl-piperazin-1-yl)-[2-(4-fluoro-benzoyl)-1,2,3,4-tetrahydro-isoquinolin-6-yl]-methanone;
(4-Cyclobutyl-piperazin-1-yl)-[2-(2,4-difluoro-benzoyl)-1,2,3,4-tetrahydro-isoquinolin-6-yl]-methanone;
(4-Cyclobutyl-piperazin-1-yl)-[2-(3-fluoro-4-methyl-benzoyl)-1,2,3,4-tetrahydro-isoquinolin-6-yl]-methanone;
[2-(3-Chloro-4-fluoro-benzoyl)-1,2,3,4-tetrahydro-isoquinolin-6-yl]-(4-cyclobutyl-piperazin-1-yl)-methanone;
1-[6-(4-Cyclobutyl-piperazine-1-carbonyl)-3,4-dihydro-1H-isoquinolin-2-yl]-2-phenyl-ethanone;
1-[6-(4-Cyclobutyl-piperazine-1-carbonyl)-3,4-dihydro-1H-isoquinolin-2-yl]-2-(4-fluoro-phenyl)-ethanone;
[2-(4-tert-Butyl-benzoyl)-1,2,3,4-tetrahydro-isoquinolin-6-yl]-(4-cyclobutyl-piperazin-1-yl)-methanone;
(4-Cyclobutyl-piperazin-1-yl)-[2-(4-cyclohexyl-benzoyl)-1,2,3,4-tetrahydro-isoquinolin-6-yl]-methanone;
(4-Chloro-phenyl)-[6-(4-cyclobutyl-[1,4]diazepane-1-carbonyl)-3,4-dihydro-1H-isoquinolin-2-yl]-methanone;
[6-(4-Cyclobutyl-[1,4]diazepane-1-carbonyl)-3,4-dihydro-1H-isoquinolin-2-yl]-(4-fluoro-phenyl)-methanone;
(3-Chloro-phenyl)-[6-(4-cyclobutyl-[1,4]diazepane-1-carbonyl)-3,4-dihydro-1H-isoquinolin-2-yl]-methanone;
[6-(4-Cyclobutyl-[1,4]diazepane-1-carbonyl)-3,4-dihydro-1H-isoquinolin-2-yl]-(2-fluoro-phenyl)-methanone;
[6-(4-Cyclobutyl-[1,4]diazepane-1-carbonyl)-3,4-dihydro-1H-isoquinolin-2-yl]-(tetrahydro-furan-3-yl)-methanone;
[6-(4-Cyclobutyl-[1,4]diazepane-1-carbonyl)-3,4-dihydro-1H-isoquinolin-2-yl]-(tetrahydro-furan-2-yl)-methanone;
1-[6-(4-Cyclobutyl-[1,4]diazepane-1-carbonyl)-3,4-dihydro-1H-isoquinolin-2-yl]-propan-1-one;
[6-(4-Cyclobutyl-[1,4]diazepane-1-carbonyl)-3,4-dihydro-1H-isoquinolin-2-yl]-(4-propyl-phenyl)-methanone;
[6-(4-Cyclobutyl-[1,4]diazepane-1-carbonyl)-3,4-dihydro-1H-isoquinolin-2-yl]-(4-fluoro-3-hydroxy-phenyl)-methanone;
[6-(4-Cyclobutyl-[1,4]diazepane-1-carbonyl)-3,4-dihydro-1H-isoquinolin-2-yl]-(3-fluoro-4-methyl-phenyl)-methanone;
[6-(4-Cyclobutyl-[1,4]diazepane-1-carbonyl)-3,4-dihydro-1H-isoquinolin-2-yl]-(2,4-dichloro-phenyl)-methanone;
[6-(4-Cyclobutyl-[1,4]diazepane-1-carbonyl)-3,4-dihydro-1H-isoquinolin-2-yl]-(2,4-difluoro-phenyl)-methanone;
(3-Chloro-4-fluoro-phenyl)-[6-(4-cyclobutyl-[1,4]diazepane-1-carbonyl)-3,4-dihydro-1H-isoquinolin-2-yl]-methanone;
[6-(4-Cyclobutyl-[1,4]diazepane-1-carbonyl)-3,4-dihydro-1H-isoquinolin-2-yl]-(3-methoxy-cyclohexyl)-methanone;
trans-[6-(4-Cyclobutyl-[1,4]diazepane-1-carbonyl)-3,4-dihydro-1H-isoquinolin-2-yl]-(4-methoxy-cyclohexyl)-methanone;
cis-[6-(4-Cyclobutyl-[1,4]diazepane-1-carbonyl)-3,4-dihydro-1H-isoquinolin-2-yl]-(4-methoxy-cyclohexyl)-methanone;
[2-(1-Isopropyl-piperidine-4-carbonyl)-1,2,3,4-tetrahydro-isoquinolin-6-yl]-morpholin-4-yl-methanone;
(S)-Cyclohexyl-[6-(2-pyrrolidin-1-ylmethyl-pyrrolidine-1-carbonyl)-3,4-dihydro-1H-isoquinolin-2-yl]-methanone;
Cyclohexyl-{6-[4-(tetrahydro-furan-2-ylmethyl)-piperazine-1-carbonyl]-3,4-dihydro-1H-isoquinolin-2-yl}-methanone;
Cyclohexyl-[6-(octahydro-pyrido[1,2-a]pyrazine-2-carbonyl)-3,4-dihydro-1H-isoquinolin-2-yl]-methanone;
Cyclohexyl-[6-(hexahydro-pyrrolo[1,2-a]pyrazine-2-carbonyl)-3,4-dihydro-1H-isoquinolin-2-yl]-methanone;
Cyclohexyl-[6-(4-dimethylamino-piperidine-1-carbonyl)-3,4-dihydro-1H-isoquinolin-2-yl]-methanone;
(R)-Cyclohexyl-[6-(3-dimethylamino-pyrrolidine-1-carbonyl)-3,4-dihydro-1H-isoquinolin-2-yl]-methanone;
(S)-Cyclohexyl-[6-(3-dimethylamino-pyrrolidine-1-carbonyl)-3,4-dihydro-1H-isoquinolin-2-yl]-methanone;
[6-([1,4′]Bipiperidinyl-1′-carbonyl)-3,4-dihydro-1H-isoquinolin-2-yl]-cyclohexyl-methanone;
Cyclohexyl-[6-(4-morpholin-4-yl-piperidine-1-carbonyl)-3,4-dihydro-1H-isoquinolin-2-yl]-methanone;
Cyclohexyl-[6-(4-cyclopentyl-piperazine-1-carbonyl)-3,4-dihydro-1H-isoquinolin-2-yl]-methanone;
Cyclohexyl-[6-(4-cyclohexyl-piperazine-1-carbonyl)-3,4-dihydro-1H-isoquinolin-2-yl]-methanone;
2-Cyclohexanecarbonyl-1,2,3,4-tetrahydro-isoquinoline-6-carboxylic acid methyl-(1-methyl-pyrrolidin-3-yl)-amide;
Cyclohexyl-[6-(4-isopropyl-[1,4]diazepane-1-carbonyl)-3,4-dihydro-1H-isoquinolin-2-yl]-methanone;
(5-Cyclobutyl-hexahydro-pyrrolo[3,4-c]pyrrol-2-yl)-(2-cyclohexanecarbonyl-1,2,3,4-tetrahydro-isoquinolin-6-yl)-methanone;
(1S,4S)-(5-Cyclobutyl-2,5-diaza-bicyclo[2.2.1]hept-2-yl)-(2-cyclohexanecarbonyl-1,2,3,4-tetrahydro-isoquinolin-6-yl)-methanone;
(1-Cyclobutyl-hexahydro-pyrrolo[3,4-b]pyrrol-5-yl)-(2-cyclohexanecarbonyl-1,2,3,4-tetrahydro-isoquinolin-6-yl)-methanone;
Cyclohexyl-(6-piperidin-1-ylmethyl-3,4-dihydro-1H-isoquinolin-2-yl)-methanone;
Cyclohexyl-(6-morpholin-4-ylmethyl-3,4-dihydro-1H-isoquinolin-2-yl)-methanone;
Cyclohexyl-[6-(octahydro-pyrido[1,2-a]pyrazin-2-ylmethyl)-3,4-dihydro-1H-isoquinolin-2-yl]-methanone;
Cyclohexyl-[6-(2-pyrrolidin-1-ylmethyl-pyrrolidin-1-ylmethyl)-3,4-dihydro-1H-isoquinolin-2-yl]-methanone;
[6-(4-Cyclobutyl-piperazin-1-ylmethyl)-3,4-dihydro-1H-isoquinolin-2-yl]-cyclohexyl-methanone;
[6-(4-Cyclobutyl-[1,4]diazepan-1-ylmethyl)-3,4-dihydro-1H-isoquinolin-2-yl]-cyclohexyl-methanone;
(2-Cyclopentyl-1,2,3,4-tetrahydro-isoquinolin-6-yl)-morpholin-4-yl-methanone;
(2-Isopropyl-1,2,3,4-tetrahydro-isoquinolin-6-yl)-piperidin-1-yl-methanone;
(2-Isopropyl-1,2,3,4-tetrahydro-isoquinolin-6-yl)-morpholin-4-yl-methanone;
(2-Isopropyl-1,2,3,4-tetrahydro-isoquinolin-6-yl)-(octahydro-pyrido[1,2-a]pyrazin-2-yl)-methanone;
(4-tert-Butyl-piperidin-1-yl)-(2-isopropyl-1,2,3,4-tetrahydro-isoquinolin-6-yl)-methanone;
(4-Cyclobutyl-[1,4]diazepan-1-yl)-(2-isopropyl-1,2,3,4-tetrahydro-isoquinolin-6-yl)-methanone;
[4-(1-Hydroxy-1-methyl-ethyl)-piperidin-1-yl]-(2-isopropyl-1,2,3,4-tetrahydro-isoquinolin-6-yl)-methanone;
Piperidin-1-yl-(2-propyl-1,2,3,4-tetrahydro-isoquinolin-6-yl)-methanone;
Morpholin-4-yl-(2-propyl-1,2,3,4-tetrahydro-isoquinolin-6-yl)-methanone;
(Octahydro-pyrido[1,2-a]pyrazin-2-yl)-(2-propyl-1,2,3,4-tetrahydro-isoquinolin-6-yl)-methanone;
(4-tert-Butyl-piperidin-1-yl)-(2-propyl-1,2,3,4-tetrahydro-isoquinolin-6-yl)-methanone;
(4-Cyclobutyl-[1,4]diazepan-1-yl)-(2-propyl-1,2,3,4-tetrahydro-isoquinolin-6-yl)-methanone;
[4-(1-Hydroxy-1-methyl-ethyl)-piperidin-1-yl]-(2-propyl-1,2,3,4-tetrahydro-isoquinolin-6-yl)-methanone;
(2-Cyclobutyl-1,2,3,4-tetrahydro-isoquinolin-6-yl)-piperidin-1-yl-methanone;
(2-Cyclobutyl-1,2,3,4-tetrahydro-isoquinolin-6-yl)-morpholin-4-yl-methanone;
(2-Cyclobutyl-1,2,3,4-tetrahydro-isoquinolin-6-yl)-(octahydro-pyrido[1,2-a]pyrazin-2-yl)-methanone;
(4-tert-Butyl-piperidin-1-yl)-(2-cyclobutyl-1,2,3,4-tetrahydro-isoquinolin-6-yl)-methanone;
(4-Cyclobutyl-[1,4]diazepan-1-yl)-(2-cyclobutyl-1,2,3,4-tetrahydro-isoquinolin-6-yl)-methanone;
(2-Cyclobutyl-1,2,3,4-tetrahydro-isoquinolin-6-yl)-[4-(1-hydroxy-1-methyl-ethyl)-piperidin-1-yl]-methanone;
(2-Cyclopentyl-1,2,3,4-tetrahydro-isoquinolin-6-yl)-piperidin-1-yl-methanone;
(2-Cyclopentyl-1,2,3,4-tetrahydro-isoquinolin-6-yl)-(octahydro-pyrido[1,2-a]pyrazin-2-yl)-methanone;
(4-tert-Butyl-piperidin-1-yl)-(2-cyclopentyl-1,2,3,4-tetrahydro-isoquinolin-6-yl)-methanone; and
(2-Cyclopentyl-1,2,3,4-tetrahydro-isoquinolin-6-yl)-[4-(1-hydroxy-1-methyl-ethyl)-piperidin-1-yl]-methanone;
and pharmaceutically acceptable salts thereof.

20. A compound as defined in claim 1, or a pharmaceutically acceptable salt thereof.

21. A pharmaceutical composition for treating a disease, disorder, or medical condition mediated by histamine H₃ receptor activity, comprising:
(a) an effective amount of a compound of Formula (I): 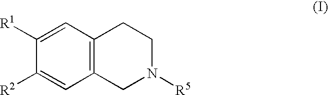
wherein
one of R¹ and R² is -L-N(R³)R⁴ and the other is —H;
where L is C(O) or CH₂; and
—N(R³)R⁴ is one of the following moieties: 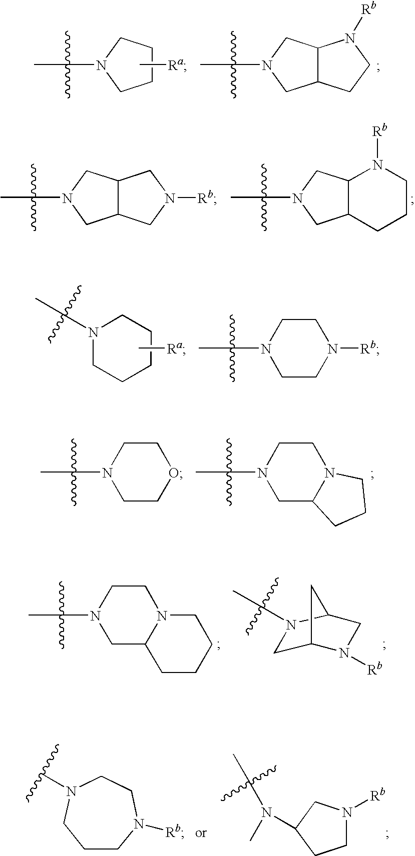
where R^{a} is —H, —C₁₋₄alkyl, —C₁₋₄alkyl-OH, —OH, —NR^{c}R^{d}, or —CH₂NR^{c}R^{d};
R^{c} and R^{d} are each independently H or —C₁₋₄alkyl, or R^{c} and R^{d} taken together with the nitrogen to which they are attached form pyrrolidinyl, piperidinyl, or morpholinyl; and
R^{b} is —C₁₋₄alkyl or —C₃₋₇cycloalkyl;
R⁵ is —H, C₁₋₄alkyl, C₃₋₇cycloalkyl, —CH₂-phenyl, —CH₂-(monocyclic heteroaryl), —C(O)—C₁₋₄alkyl, —C(O)—C₃₋₇cycloalkyl, —C(O)-(monocyclic heterocycloalkyl), —C(O)-phenyl, —C(O)-(monocyclic heteroaryl), —C(O)CH₂—C₃₋₇cycloalkyl, —C(O)CH₂-phenyl, —C(O)CH₂-(monocyclic heteroaryl), —CO₂C₁₋₄alkyl, —SO₂C₁₋₄alkyl, or —SO₂-phenyl;
where each cycloalkyl, phenyl, monocyclic heteroaryl, or monocyclic heterocycloalkyl group in R⁵ is unsubstituted or substituted with one or two substituents independently selected from the group consisting of —C₁₋₄alkyl, —CF₃, halo, —CN, —NO₂, —OH, —OC₁₋₄alkyl, —C₃₋₇cycloalkyl, and —NR^{x}R^{y};
R^{x} and R^{y} are each independently H or —C₁₋₄alkyl;
with the proviso that the compound of Formula (I) comprises at least one nitrogen atom which is not part of an amide, carbamoyl, cyano, nitro, or sulfonamide group;
or a pharmaceutically acceptable salt, pharmaceutically acceptable prodrug, or pharmaceutically active metabolite thereof; and
(b) a pharmaceutically acceptable excipient.

22. A pharmaceutical composition according to claim 21, further comprising: an active ingredient selected from the group consisting of H₁ receptor antagonists, H₂ receptor antagonists, H₃ receptor antagonists, serotonin-norepinephrine reuptake inhibitors, selective serotonin reuptake inhibitors, noradrenergic reuptake inhibitors, non-selective serotonin re-uptake inhibitors, acetylcholinesterase inhibitors, and modafinil.

23. A pharmaceutical composition according to claim 21, further comprising topiramate.

24. A method of treating a subject suffering from or diagnosed with a disease, disorder, or medical condition mediated by histamine H₃ receptor activity, comprising administering to the subject in need of such treatment an effective amount of a compound of Formula (I): 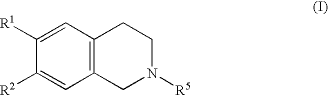
wherein
one of R¹ and R² is -L-N(R³)R⁴ and the other is —H;
where L is C(O) or CH₂; and
—N(R³)R⁴ is one of the following moieties: 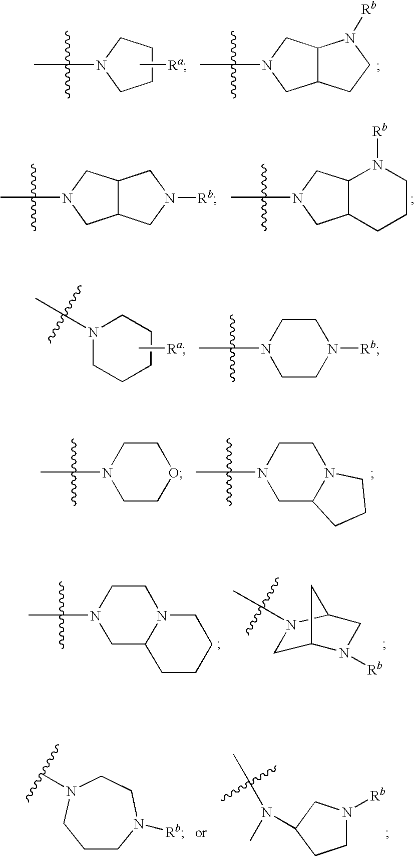
where R^{a} is —H, —C₁₋₄alkyl, —C₁₋₄alkyl-OH, —OH, —NR^{c}R^{d}, or —CH₂NR^{c}R^{d};
R^{c} and R^{d} are each independently H or —C₁₋₄alkyl, or R^{c} and R^{d} taken together with the nitrogen to which they are attached form pyrrolidinyl, piperidinyl, or morpholinyl; and
R^{b} is —C₁₋₄alkyl or —C₃₋₇cycloalkyl;
R⁵ is —H, C₁₋₄alkyl, C₃₋₇cycloalkyl, —CH₂-phenyl, —CH₂-(monocyclic heteroaryl), —C(O)—C₁₋₄alkyl, —C(O)—C₃₋₇cycloalkyl, —C(O)-(monocyclic heterocycloalkyl), —C(O)-phenyl, —C(O)-(monocyclic heteroaryl), —C(O)CH₂—C₃₋₇cycloalkyl, —C(O)CH₂-phenyl, —C(O)CH₂-(monocyclic heteroaryl), —CO₂C₁₋₄alkyl, —SO₂C₁₋₄alkyl, or —SO₂-phenyl;
where each cycloalkyl, phenyl, monocyclic heteroaryl, or monocyclic heterocycloalkyl group in R⁵ is unsubstituted or substituted with one or two substituents independently selected from the group consisting of —C₁₋₄alkyl, —CF₃, halo, —CN, —NO₂, —OH, —OC₁₋₄alkyl, —C₃₋₇cycloalkyl, and —NR^{x}R^{y};
R^{x} and R^{y} are each independently H or —C₁₋₄alkyl;
with the proviso that the compound of Formula (I) comprises at least one nitrogen atom which is not part of an amide, carbamoyl, cyano, nitro, or sulfonamide group;
or a pharmaceutically acceptable prodrug, or pharmaceutically active metabolite thereof.

25. The method according to claim 24, wherein the disease, disorder, or medical condition is selected from the group consisting of: cognitive disorders, sleep disorders, psychiatric disorders, and other disorders.

26. The method according to claim 25, wherein the disease, disorder, or medical condition is selected from the group consisting of: dementia, Alzheimer's disease, cognitive dysfunction, mild cognitive impairment, pre-dementia, attention deficit hyperactivity disorders, attention-deficit disorders, and learning and memory disorders.

27. The method according to claim 24, wherein the disease, disorder, or medical condition is selected from the group consisting of: learning impairment, memory impairment, age-related cognitive decline, and memory loss.

28. The method according to claim 24, wherein the disease, disorder, or medical condition is selected from the group consisting of: insomnia, disturbed sleep, narcolepsy with or without associated cataplexy, cataplexy, disorders of sleep/wake homeostasis, idiopathic somnolence, excessive daytime sleepiness, circadian rhythm disorders, fatigue, lethargy, jet lag and REM-behavioral disorder.

29. The method according to claim 24, wherein the disease, disorder, or medical condition is selected from the group consisting of: sleep apnea, perimenopausal hormonal shifts, Parkinson's disease, multiple sclerosis, depression, chemotherapy, and shift work schedules.

30. The method according to claim 24, wherein the disease, disorder, or medical condition is selected from the group consisting of: schizophrenia, bipolar disorders, manic disorders, depression, obsessive-compulsive disorder, and post-traumatic stress disorder.

31. The method according to claim 24, wherein the disease, disorder, or medical condition is selected from the group consisting of: motion sickness, vertigo, benign postural vertigo, tinitus, epilepsy, migraine, neurogenic inflammation, neuropathic pain, Down Syndrome, seizures, eating disorders, obesity, substance abuse disorders, movement disorders, restless legs syndrome, eye-related disorders, macular degeneration, and retinitis pigmentosis.

32. The method according to claim 24, wherein the disease, disorder, or medical condition is selected from the group consisting of: depression, disturbed sleep, fatigue, lethargy, cognitive impairment, memory impairment, memory loss, learning impairment, attention-deficit disorders, and eating disorders.
